(19) United States
(12) Patent Application Publication
(10) Pub. No.: US 2026/0272975 A1
(43) Pub. Date: Sep. 17, 2026

(54) BIOPOLYMERIC ANTI-ANTIBIOTICS AND METHODS FOR MAKING AND USING THE SAME

(71) Applicant: The Penn State Research Foundation, University Park, PA (US)

(72) Inventors: Amir Sheikhi, University Park, PA (US); Roya Koshani, University Park, PA (US)

(73) Assignee: The Penn State Research Foundation, University Park, PA (US)

(21) Appl. No.: 19/552,097

(22) Filed: Feb. 27, 2026

Related U.S. Application Data
(63) Continuation-in-part of application No. PCT/US2025/013776, filed on Jan. 30, 2025.
(60) Provisional application No. 63/776,088, filed on Jan. 1, 0001.
(60) Provisional application No. 63/627,259, filed on Jan. 31, 2024.
(60) Provisional application No. 63/647,745, filed on May 15, 2024.

(51) Int. Cl. A61K 31/717; A61K 9/51; A61P 43/00

(52) U.S. Cl. CPC A61K 31/717; A61K 9/5146; A61P 43/00

(57) ABSTRACT

A process and system for removal of antibiotic compounds from a patient's body can include having a patient digest an antibiotic removal agent or an antibiotic deactivation agent. The agent can be synthesized from a method including the steps of providing hairy cellulose nanocrystals or functionalized biopolymers; adsorbing a target antibiotic compound onto the hairy cellulose nanocrystals or biopolymer via electrostatic interactions; polymerizing a zwitterionic polymer around the antibiotic-adsorbed hairy cellulose nanocrystals or biopolymers; and removing the target antibiotic compound via a surfactant or solvent to form the antibiotic removal agent.

### CROSS-REFERENCE TO RELATED APPLICATIONS

The present application is a continuation-in-part application of International Patent Application No. PCT/US2025/013776, filed on Jan. 30, 2025, which is related to and claims priority to U.S. Provisional Patent Application No. 63/627,259, filed on Jan. 31, 2024, and to U.S. Provisional Patent Application No. 63/647,745, filed on May 15, 2024. The present application is further related to and claims priority to U.S. Provisional Patent Application No. 63/776,008, filed on Mar. 3, 2025. The entire contents of these applications are incorporated by reference.

### STATEMENT REGARDING FEDERALLY SPONSORED RESEARCH DEVELOPMENT

This invention was made with government support under Grant No. NI24HFPXXXXXG033 awarded by the United States Department of Agriculture. The Government has certain rights in the invention.

### FIELD

Embodiments relate to processes and apparatus configured to reduce, hinder, or avoid the growth or development of drug resistant bacteria and/or the protection of drug sensitive bacteria. Embodiments can be adapted to help block or avoid antibiotic-resistant bacterial growth within the body of a patient, for example. Embodiments can be adapted to help protect or promote antibiotic-sensitive bacterial growth within the body of a patient, for example. Embodiments can be adapted to help protect the microbiome within the body of a patient, for example.

### BACKGROUND

To combat bacterial infections in humans, antibiotics have extensively been used to save millions of lives since the 20^{th} century. Vancomycin (VAN), a glycopeptide antibiotic, isolated from the fermentation of a soil actinomycete, has been a key drug in treating Gram-positive pathogen infections, such as methicillin-resistant Staphylococcus aureus (MRSA). VAN inhibits bacterial cell wall biosynthesis through binding to the D-alanyl-D-alanine moieties of peptidoglycan layer in the bacteria cell wall. This binding occurs via five hydrogen bonds between VAN heptapeptide and the D-alanyl-D-alanine, as confirmed by the NMR spectroscopy. Accordingly, VAN has been one of the most effective regimens for Gram-positive pathogen infections.

In clinical treatments, VAN is usually administered intravenously to patients infected by Gram-positive pathogens. Excess VAN in the bloodstream is mainly eliminated by the kidneys; however, biliary excretion leads to the bioavailability of ~5-10% of intravenous VAN in the intestines, which has been confirmed by measuring up to 100 μg of VAN per mL in human stools. The intestine-exposed VAN may drive the VAN-susceptible Enterococcus faecium (VSEfm), a common colonizer, to gain resistance. VAN-resistant E. faecium (VREfm) may enter the bloodstream via moving across the intestinal lining and passing through the liver, which potentially causes infective endocarditis when contacting the heart. Additionally, environmental fecal exposure may lead to the VRE transmission to other patients via contaminating the medical equipment or communal surfaces in hospitals.

### SUMMARY

We hypothesize that capturing off-target VAN in the GI tract may prevent the evolution and transmission of VREs. So far, a few approaches have been investigated to protect the gut microbiome via capturing or deactivating antibiotics, including the oral administration of D-alanyl-D-alanine-functionalized polyethylene glycol microparticles and D-alanyl-D-alanine-based antagonists. However, the building blocks of D-alanyl-D-alanine-functionalized microparticles comprise L-amino acids, in addition to D-alanyl-D-alanine, which are susceptible to enzymatic degradation in the GI tract. Additionally, in vivo studies were conducted on mice orally treated with high concentrations of VAN, conditions that do not reflect clinical scenarios wherein VAN is administered intravenously. Among the peptide libraries mimicking the bacterial VAN binding site, i.e., D-alanyl-D-alanine, a few analogs could neutralize VAN activity against Enterococci, but only at high peptide:VAN molar ratios. For example, acetyl-lysine (acetyl)-D-alanyl-D-alanine was effective at a peptide:VAN ratio of 28:1. In addition, the analogs were more effective at high VAN concentrations (e.g., 32 μg mL⁻¹), compared with low VAN concentrations (e.g., 8 μg mL⁻¹), which is closer to the physiological GI-exposed VAN concentration. Accordingly, translating the current peptide-enabled analogs to in vivo applications is challenging.

We have developed a novel anti-VAN biomaterial via hybridizing hairy cellulose nanocrystals with a Food and Drug Administration (FDA)-approved resin to remove the excess antibiotic from the GI tract before it impacts bacteria and selects for resistance. In vitro studies show that VAN removal is regulated by electrostatic interactions via a time-dependent diffusion-controlled process that is not significantly influenced by the physiological pH, ionic strength, or the components of simulated intestinal fluid. Aligned with the in vitro findings, the oral administration of anti-VAN adjuvant may effectively sequester VAN in the murine GI tract and prevented the VAN resistance enrichment following the VAN treatment of E. faecium colonized mice. The anti-VAN biomaterial may protect intravenous VAN, which can be a step forward in addressing the global threat of antimicrobial resistance.

In an exemplary embodiment, an antibiotic removal or deactivation agent comprises hairy cellulose nanocrystals; a particulate resin; and optionally, a functionalized biopolymer, wherein the hairy cellulose nanocrystals are immobilized on the particulate resin.

In some embodiments, the particulate resin is comprised of cholestyramine, patiromer, sodium polystyrene sulfonate, sodium zirconium cyclosilicate, sodium polystyrene sulfonate, sodium polystyrene sulfonate, colestipol, tolevamer, polacrilex resin (Amberlite IRP64), polacrilin potassium (Amberlite IRP88), colesevelam, sevelamer (SEV), magnesium-containing compounds, calcium-containing compounds, iron-containing compounds, zinc-containing compounds, antacids containing aluminum or magnesium, proton pump inhibitors, or combinations thereof.

In some embodiments, the particulate resin is cholestyramine.

In some embodiments, the hairy cellulose nanocrystals comprise cellulose bodies bearing cellulose chains protruding therefrom, wherein the cellulose chains are functionalized with anionic groups.

In some embodiments, the anionic groups comprise carboxylate groups.

In some embodiments, the hairy cellulose nanocrystals comprise cellulose bodies bearing cellulose chains protruding therefrom, wherein the cellulose chains are functionalized with cationic groups.

In some embodiments, the hairy cellulose nanocrystals comprise cellulose bodies bearing cellulose chains protruding therefrom, wherein the cellulose chains are functionalized with amphiphilic groups.

In some embodiments, the hairy cellulose nanocrystals comprise cellulose bodies bearing cellulose chains protruding therefrom, wherein the cellulose chains are functionalized with zwitterionic groups.

In some embodiments, the hairy cellulose nanocrystals comprise cellulose bodies bearing cellulose chains protruding therefrom, wherein the cellulose chains are functionalized with a combination of electrically neutral anionic, cationic, zwitterionic, and/or amphiphilic groups.

In some embodiments, the functionalized biopolymer comprises polysaccharide, protein, polynucleotides, and/or polypeptides, wherein the biopolymer is functionalized with a combination of electrically neutral anionic, cationic, zwitterionic, and/or amphiphilic groups.

In some embodiments, the agent is configured to be administered to a gastrointestinal (GI) tract of a patient, such that when the GI tract includes one or more antibiotic compounds, the agent is configured to effectively remove the one or more antibiotic compounds.

In some embodiments, the one or more antibiotic compounds include B-lactams, aminoglycosides, chloramphenicol, glycopeptides, quinolones, oxazolidinones, sulfonamides, tetracyclines, macrolides, ansamycins, streptogramins, lipopeptides, or combinations thereof.

In some embodiments, the one or more antibiotic compounds include Vancomycin (VAN).

In an exemplary embodiment, an antibiotic removal or deactivation agent comprises a functionalized biopolymer, wherein the functionalized biopolymer comprises polysaccharide, protein, polynucleotides, and/or polypeptides, wherein the polymer is functionalized with a combination of electrically neutral anionic, cationic, zwitterionic, and/or amphiphilic groups.

In some embodiments, the agent is configured to be administered to a gastrointestinal (GI) tract of a patient, such that when the GI tract includes one or more antibiotic compounds, the agent is configured to effectively remove the one or more antibiotic compounds.

In some embodiments, the one or more antibiotic compounds include B-lactams, aminoglycosides, chloramphenicol, glycopeptides, quinolones, oxazolidinones, sulfonamides, tetracyclines, macrolides, ansamycins, streptogramins, lipopeptides, or combinations thereof.

In some embodiments, the one or more antibiotic compounds include Vancomycin (VAN).

In an exemplary embodiment, a process for removing one or more antibiotic compounds from a gastrointestinal (GI) tract of a patient comprises administering an antibiotic removal or deactivation agent to the patient for digestion so that the antibiotic removal agent or the antibiotic deactivation agent is present in the GI tract of the patient, wherein the antibiotic removal or deactivation agent effectively removes the one or more antibiotic compounds present in the GI tract, and wherein the antibiotic removal or deactivation agent comprises hairy cellulose nanocrystals immobilized on a particulate resin.

In some embodiments, the particulate resin is comprised of cholestyramine, patiromer, sodium polystyrene sulfonate, sodium zirconium cyclosilicate, sodium polystyrene sulfonate, sodium polystyrene sulfonate, colestipol, tolevamer, polacrilex resin (Amberlite IRP64), polacrilin potassium (Amberlite IRP88), colesevelam, sevelamer (SEV), magnesium-containing compounds, calcium-containing compounds, iron-containing compounds, zinc-containing compounds, antacids containing aluminum or magnesium, proton pump inhibitors, or combinations thereof.

In some embodiments, the particulate resin comprises cholestyramine.

In some embodiments, the one or more antibiotic compounds include B-lactams, aminoglycosides, chloramphenicol, glycopeptides, quinolones, oxazolidinones, sulfonamides, tetracyclines, macrolides, ansamycins, streptogramins, lipopeptides, or combinations thereof.

In some embodiments, the one or more antibiotic compounds include Vancomycin (VAN).

In some embodiments, the antibiotic removal or deactivation agent effectively removing the one or more antibiotic compounds present in the GI tract includes the antibiotic removal or deactivation agent interacting with the one or more antibiotic compounds present in the GI tract such that one or more antibacterial compounds present in the GI tract are non-bioreactive with bacteria within the GI tract.

In some embodiments, the process further comprises treating the patient with the one or more antibiotic compounds to treat an infectious disease in the patient.

In some embodiments, the infectious disease is a bacterial infection that is not located in the GI tract.

In some embodiments, the infectious disease is a bacterial infection that is located in the GI tract.

In some embodiments, the process further comprises treating the patient with the one or more antibiotic compounds to treat a non-infectious disease in the patient.

In some embodiments, the non-infectious disease is a bacterial infection that is not located in the GI tract.

In some embodiments, the non-infectious disease is a bacterial infection that is located in the GI tract.

In some embodiments, the administering of the antibiotic removal or deactivation agent to the patient for the patient to digest so that the antibiotic removal or deactivation agent is present in the GI tract of the patient comprises feeding the patient the antibiotic removal or deactivation agent such that the antibiotic removal or deactivation agent is orally consumed by the patient.

In some embodiments, the administering of the antibiotic removal or deactivation agent to the patient for the patient to digest so that the antibiotic removal or deactivation agent is present in the GI tract of the patient comprises feeding the patient the antibiotic removal or deactivation agent periodically while the patient undergoes a treatment for an infection that includes multiple injections of the one or more antibiotic compounds into the patient at different spaced apart intervals.

In some embodiments, the administering is performed periodically different times a day for a number of days.

In some embodiments, the antibiotic removal or deactivation agent effectively removes the one or more antibiotic compounds present in the GI tract such that bacteria within the GI tract is unaffected by the antibiotic compounds.

In some embodiments, the antibiotic removal or deactivation agent effectively removes the one or more antibiotic compounds present in the GI tract to reduce, hinder, or avoid the growth or development of drug resistant bacteria and/or the protection of drug sensitive bacteria.

In some embodiments, the antibiotic removal or deactivation agent effectively removes the one or more antibiotic compounds present in the GI tract to block or avoid antibiotic-resistant bacterial growth within the body.

In some embodiments, the antibiotic removal or deactivation agent effectively removes the one or more antibiotic compounds present in the GI tract to help protect or promote antibiotic-sensitive bacterial growth within the body.

In some embodiments, the antibiotic removal or deactivation agent effectively removes the one or more antibiotic compounds present in the GI tract to help protect the microbiome within the body.

In an exemplary embodiment, a process for removing one or more antibiotic compounds from a gastrointestinal (GI) tract of a patient comprises administering an antibiotic removal or deactivation agent to the patient for digestion so that the antibiotic removal agent or the antibiotic deactivation agent is present in the GI tract of the patient, wherein the antibiotic removal or deactivation agent effectively removes the one or more antibiotic compounds present in the GI tract, and wherein the antibiotic removal or deactivation agent comprises a functionalized biopolymer, wherein the functionalized biopolymer comprises polysaccharide, protein, polynucleotides, and/or polypeptides, wherein the polymer is functionalized with a combination of electrically neutral anionic, cationic, zwitterionic, and/or amphiphilic groups.

In some embodiments, the particulate resin is comprised of cholestyramine, patiromer, sodium polystyrene sulfonate, sodium zirconium cyclosilicate, sodium polystyrene sulfonate, sodium polystyrene sulfonate, colestipol, tolevamer, polacrilex resin (Amberlite IRP64), polacrilin potassium (Amberlite IRP88), colesevelam, sevelamer (SEV), magnesium-containing compounds, calcium-containing compounds, iron-containing compounds, zinc-containing compounds, antacids containing aluminum or magnesium, proton pump inhibitors, or combinations thereof.

In some embodiments, the particulate resin comprises cholestyramine.

In some embodiments, the one or more antibiotic compounds include B-lactams, aminoglycosides, chloramphenicol, glycopeptides, quinolones, oxazolidinones, sulfonamides, tetracyclines, macrolides, ansamycins, streptogramins, lipopeptides, or combinations thereof.

In some embodiments, the one or more antibiotic compounds include Vancomycin (VAN).

In some embodiments, the antibiotic removal or deactivation agent effectively removing the one or more antibiotic compounds present in the GI tract includes the antibiotic removal or deactivation agent interacting with the one or more antibiotic compounds present in the GI tract such that one or more antibacterial compounds present in the GI tract are non-bioreactive with bacteria within the GI tract.

In some embodiments, the process further comprises treating the patient with the one or more antibiotic compounds to treat an infectious disease in the patient.

In some embodiments, the infectious disease is a bacterial infection that is not located in the GI tract.

In some embodiments, the infectious disease is a bacterial infection that is located in the GI tract.

In some embodiments, the process further comprises treating the patient with the one or more antibiotic compounds to treat a non-infectious disease in the patient.

In some embodiments, the non-infectious disease is a bacterial infection that is not located in the GI tract.

In some embodiments, the non-infectious disease is a bacterial infection that is located in the GI tract.

In some embodiments, the administering of the antibiotic removal or deactivation agent to the patient for the patient to digest so that the antibiotic removal or deactivation agent is present in the GI tract of the patient comprises feeding the patient the antibiotic removal or deactivation agent such that the antibiotic removal or deactivation agent is orally consumed by the patient.

In some embodiments, the administering of the antibiotic removal or deactivation agent to the patient for the patient to digest so that the antibiotic removal or deactivation agent is present in the GI tract of the patient comprises feeding the patient the antibiotic removal or deactivation agent periodically while the patient undergoes a treatment for an infection that includes multiple injections of the one or more antibiotic compounds into the patient at different spaced apart intervals.

In some embodiments, the administering is performed periodically different times a day for a number of days.

In some embodiments, the antibiotic removal or deactivation agent effectively removes the one or more antibiotic compounds present in the GI tract such that bacteria within the GI tract is unaffected by the antibiotic compounds.

In some embodiments, the antibiotic removal or deactivation agent effectively removes the one or more antibiotic compounds present in the GI tract to reduce, hinder, or avoid the growth or development of drug resistant bacteria and/or the protection of drug sensitive bacteria.

In some embodiments, the antibiotic removal or deactivation agent effectively removes the one or more antibiotic compounds present in the GI tract to block or avoid antibiotic-resistant bacterial growth within the body.

In some embodiments, the antibiotic removal or deactivation agent effectively removes the one or more antibiotic compounds present in the GI tract to help protect or promote antibiotic-sensitive bacterial growth within the body.

In some embodiments, the antibiotic removal or deactivation agent effectively removes the one or more antibiotic compounds present in the GI tract to help protect the microbiome within the body.

In an exemplary embodiment, a method of forming an antibiotic removal agent includes providing hairy cellulose nanocrystals; adsorbing a target antibiotic compound onto the hairy cellulose nanocrystals via electrostatic interactions; polymerizing a zwitterionic polymer around the antibiotic-adsorbed hairy cellulose nanocrystals; and removing the target antibiotic compound via a surfactant or solvent to form the antibiotic removal agent.

In some embodiments, the hairy cellulose nanocrystals comprise cellulose bodies bearing cellulose chains protruding therefrom, wherein the cellulose chains are functionalized with anionic groups.

In some embodiments, the anionic groups comprise carboxylate groups.

In some embodiments, the hairy cellulose nanocrystals comprise cellulose bodies bearing cellulose chains protruding therefrom, wherein the cellulose chains are functionalized with cationic groups.

In some embodiments, the hairy cellulose nanocrystals comprise cellulose bodies bearing cellulose chains protruding therefrom, wherein the cellulose chains are functionalized with amphiphilic groups.

In some embodiments, the hairy cellulose nanocrystals comprise cellulose bodies bearing cellulose chains protruding therefrom, wherein the cellulose chains are functionalized with zwitterionic groups.

In some embodiments, the agent is configured to be administered to a gastrointestinal (GI) tract of a patient, such that when the GI tract includes the target antibiotic compound, the agent is configured to effectively remove the one or more antibiotic compounds.

In some embodiments, the target antibiotic compound is selected from the group consisting of include B-lactams, aminoglycosides, chloramphenicol, glycopeptides, quinolones, oxazolidinones, sulfonamides, tetracyclines, macrolides, ansamycins, streptogramins, lipopeptides, and combinations thereof.

In some embodiments, the target antibiotic compound is Vancomycin (VAN).

In some embodiments, the zwitterionic polymer is poly(sulfobetaine) (pSBAE).

In an exemplary embodiment, a method of forming an antibiotic removal agent includes providing a functionalized biopolymer, wherein the functionalized biopolymer is selected from the group consisting of polysaccharide, protein, polynucleotides, polypeptides, and mixtures thereof; adsorbing a target antibiotic compound onto the functionalized biopolymer via electrostatic interactions; polymerizing a zwitterionic polymer around the antibiotic-adsorbed biopolymer; and removing the target antibiotic compound via a surfactant or solvent to form the antibiotic removal agent.

In some embodiments, the biopolymer is functionalized with anionic groups.

In some embodiments, the anionic groups comprise carboxylate groups.

In some embodiments, the agent is configured to be administered to a gastrointestinal (GI) tract of a patient, such that when the GI tract includes the target antibiotic compound, the agent is configured to effectively remove the one or more antibiotic compounds.

In some embodiments, the target antibiotic compound is selected from the group consisting of include B-lactams, aminoglycosides, chloramphenicol, glycopeptides, quinolones, oxazolidinones, sulfonamides, tetracyclines, macrolides, ansamycins, streptogramins, lipopeptides, and combinations thereof.

In some embodiments, the target antibiotic compound is Vancomycin (VAN).

In some embodiments, the zwitterionic polymer is poly(sulfobetaine) (pSBAE).

Further features, aspects, objects, advantages, and possible applications of the present invention will become apparent from a study of the exemplary embodiments and examples described below, in combination with the Figures, and the appended claims.

### BRIEF DESCRIPTION OF THE FIGURES

The above and other objects, aspects, features, advantages, and possible applications of the present invention will be more apparent from the following more particular description thereof, presented in conjunction with the following drawings. It should be understood that like reference numbers used in the drawings may identify like components.

FIG. 1 is a schematic illustration of an exemplary embodiment of out anti-antibiotic process. The removal of intestine-exposed antibiotics using anti-antibiotics may prevent the emergence of resistant bacteria; however, in the absence of any anti-antibiotics, antibiotic resistance evolution following intravenous VAN injection and excretion into the intestines may lead to the emergence of resistant bacteria that may enter the bloodstream and cause infections.

FIG. 2 is a schematic illustration of an exemplary process for nanoengineering of cellulose fibrils via successive oxidation reactions, yielding anionic hairy cellulose nanocrystals (AHCNC).

FIG. 3 is a representative atomic force microscopy (AFM) image of AHCNC, showing their needle-like crystalline body.

FIG. 4 is a graph illustrating an aldehyde content of dialdehyde-modified cellulose (DAMC). Titrations were performed using a 10 mM NaOH solution.

FIG. 5 is a graph illustrating a carboxylate content of AHCNC. Titrations were performed using a 10 mM NaOH solution.

FIG. 6 is a graph illustrating an AHCNC-mediated vancomycin (VAN) (C_{0,VAN}=6 mg mL⁻¹) removal percentage (R) and capacity (qₑ) versus incubation time. AHCNC dispersion concentration and total volume were 0.25 mg mL⁻¹ and 1 mL, respectively. pH was adjusted to 6.5 using a NaOH solution (0.5 M).

FIG. 7 is a graph illustrating R at varying C_{0,VAN}. AHCNC dispersion concentration and total volume were 0.25 mg mL⁻¹ and 1 mL, respectively. pH was adjusted to 6.5 using a NaOH solution (0.5 M).

FIG. 8 is a graph illustrating qₑ at varying equilibrium VAN concentrations (C_{e,VAN}). AHCNC dispersion concentration and total volume were 0.25 mg mL⁻¹ and 1 mL, respectively. pH was adjusted to 6.5 using a NaOH solution (0.5 M).

FIG. 9 is a graph illustrating ζ-potential of AHCNC (0.1% w/v) at varying C_{0,VAN}, showing a decrease by increasing C_{0,VAN} as a result of electrostatic interactions between AHCNC and VAN, causing charge neutralization. The schematics of nanorods represent AHCNC, and the clusters are VAN molecules, showing the saturation of AHCNC carboxylate groups on the protruding hairs by increasing C_{0,VAN}. PH was adjusted to 6.5 using a NaOH solution (0.5 M).

FIG. 10 is a graph illustrating hydrodynamic equivalent size of AHCNC (0.1% w/v) at varying C_{0,VAN}. The schematics of nanorods represent AHCNC, and the clusters are VAN molecules, showing the saturation of AHCNC carboxylate groups on the protruding hairs by increasing C_{0,VAN}. pH was adjusted to 6.5 using a NaOH solution (0.5 M).

FIG. 11 is a schematic illustration of the effect of VAN on AHCNC colloidal behavior at an initial VAN concentration (C_{0,VAN}) below or above the charge stoichiometry ratio.

FIG. 12 is a schematic illustration of the formation of AHCHC-CHA anti-antibiotics via electrostatic interactions.

FIG. 13 shows optical microscopy images of cholestyramine (CHA) and AHCNC-CHA particles sedimented in ultrapure water.

FIG. 14 is a graph illustrating the effect of AHCNC-to-CHA mass ratio, shown based on varying molar ratios (MR) of AHCNC COO⁻:CHA N⁺(CH₃)₃, on the AHCNC-CHA carboxylate group content and the mass ratio of adsorbed AHCNC (M_{A, AHCNC}) with respect to CHA mass (m_{CHA}).

FIG. 15 is a schematic illustration of VAN chemical structure and its removal process by AHCNC-CHA anti-antibiotics.

FIG. 16 is a graph illustrating VAN removal percentage (R) of AHCNC-CHA with varying formulations, indicating R~95% at the AHCNC-CHA MR above 6, bearing the highest carboxylate group content to interact with VAN amine groups.

FIG. 17 is a graph illustrating R for varying AHCNC-CHA formulations after 4 h of VAN incubation with AHCNC-CHA in aqueous solutions with varying pH, showing a decrease in R by decreasing the pH.

FIG. 18 is a graph illustrating mass ratio of desorbed AHCNC (m_{D,AHCNC}) from AHCNC-CHA with respect to AHCNC-CHA mass (m_{AHCNC-CHA}) after 6 h of incubation in aqueous solutions with varying pH. The AHCNC-CHA formulated with varying COO⁻:(N⁺(CH₃)₃) MR are shown as AHCNC-CHA-MRn (n=2, 4, 6, 8, 12).

FIG. 19 is a graph illustrating VAN (1 mg mL⁻¹) removal percentage (R) of AHCNC-CHA anti-antibiotic at varying doses after 6 h of AHCNC-CHA pre-removal incubation at pH=1.5. The data within the black dashed bracket shows that the equilibrium VAN concentration was lower than the detection limit of UV-vis spectrophotometer, thus R~95% is likely underestimated.

FIG. 20 shows scanning electron microscope (SEM) images of CHA and AHCNC-CHA.

FIG. 21 is a table including surface area, pore volume, and average pore diameter of CHA and AHCNC-CHA.

FIG. 22 is a ATR-FTIR spectra of AHCNC, CHA, and AHCNC-CHA.

FIG. 23 is a graph illustrating R versus incubation time, showing a time-dependent VAN adsorption process as a result of AHCNC-CHA porous structure. AHCNC-CHA represents AHCNC-CHA-MR6. The data within the dashed brackets show that the equilibrium VAN concentration was lower than the detection limit of UV-vis spectrophotometer, thus R~95% is likely underestimated.

FIG. 24 is a graph illustrating R versus AHCNC-CHA dose, showing that VAN removal is dose-dependent. AHCNC-CHA represents AHCNC-CHA-MR6. The data within the dashed brackets show that the equilibrium VAN concentration was lower than the detection limit of UV-vis spectrophotometer, thus R~95% is likely underestimated.

FIG. 25 is a graph illustrating the ratio of m_{D,AHCNC} to m_{AHCNC-CHA} after incubating AHCNC-CHA in the SGF for varying periods. AHCNC-CHA represents AHCNC-CHA-MR6.

FIG. 26 is a graph illustrating R of AHCNC-CHA after 4 h of incubation in SGF versus varying AHCNC-CHA doses, indicating a decrease in R at 20 mg mL⁻¹ of AHCNC-CHA, which is compensated by increasing AHCNC-CHA dose. In all experiments, C_{0,VAN}=1 mg mL⁻¹. AHCNC-CHA represents AHCNC-CHA-MR6. The data within the dashed brackets show that the equilibrium VAN concentration was lower than the detection limit of UV-vis spectrophotometer, thus R~95% is likely underestimated.

FIG. 27 is a graph illustrating the effect of initial VAN concentration (C_{0,VAN}) on VAN removal percentage (R) and capacity (qₑ) of AHCNC-CHA after 4 h of incubation. AHCNC-CHA dose was 20 mg mL⁻¹, and the total sample volume was 1 mL.

FIG. 28 is a graph illustrating R at varying pH (C_{0,VAN}=1 mg mL⁻¹). AHCNC-CHA dose was 20 mg mL⁻¹, and the total sample volume was 1 mL.

FIG. 29 is a schematic illustration and table showing the chemical structure of VAN, the pKₐ values of its major functional groups, and the net charge of VAN and AHCNC at varying pH.

FIG. 30 is a graph illustrating ζ-potentials of VAN and AHCNC at varying pH. C_{0,VAN}=1 mg mL⁻¹ and AHCNC dose=mg mL⁻¹.

FIG. 31 is a graph illustrating hydrodynamic equivalent size of VAN and AHCNC at varying pH. C_{0,VAN}=1 mg mL⁻¹ and AHCNC dose=mg mL⁻¹.

FIG. 32 is a graph illustrating R at varying Na⁺ concentrations (C_{0,VAN}=1 mg mL⁻¹). AHCNC-CHA dose was 20 mg mL⁻¹, and the total sample volume was 1 mL. The data within the dashed bracket show that the equilibrium VAN concentration was lower than the detection limit of UV-vis spectrophotometer, thus R~95% is likely underestimated.

FIG. 33 is a graph illustrating ζ-potentials of VAN and AHCNC at varying Na⁺ concentrations. C_{0,VAN}=1 mg mL⁻¹ and AHCNC dose=mg mL⁻¹.

FIG. 34 is a graph illustrating hydrodynamic equivalent size of VAN and AHCNC at varying Na⁺ concentrations. C_{0,VAN}=1 mg mL⁻¹ and AHCNC dose=mg mL⁻¹.

FIG. 35 is a graph illustrating R at varying Ca²⁺ concentrations (C_{0,VAN}=1 mg mL⁻¹). AHCNC-CHA dose was 20 mg mL⁻¹, and the total sample volume was 1 mL. The data within the dashed bracket show that the equilibrium VAN concentration was lower than the detection limit of UV-vis spectrophotometer, thus R~95% is likely underestimated.

FIG. 36 is a graph illustrating ζ-potentials of VAN and AHCNC at varying Ca²⁺ concentrations. C_{0,VAN}=1 mg mL⁻¹ and AHCNC dose=mg mL⁻¹.

FIG. 37 is a graph illustrating hydrodynamic equivalent size of VAN and AHCNC at varying Ca²⁺ concentrations. C_{0,VAN}=1 mg mL⁻¹ and AHCNC dose=mg mL⁻¹.

FIG. 38 is a graph illustrating R at 5 mM of Ca²⁺ and varying AHCNC-CHA doses. C_{0,VAN}=1 mg mL⁻¹. The data within the dashed bracket show that the equilibrium VAN concentration was lower than the detection limit of UV-vis spectrophotometer, thus R~95% is likely underestimated.

FIG. 39 is a graph illustrating AHCNC-CHA-mediated VAN (C_{0,VAN}=1 mg mL⁻¹) removal percentage (R) at varying concentrations of bile acid. AHCNC-CHA dose was 20 mg mL⁻¹, and total sample volume=1 mL. In the graph, BA stands for bile acid. The data within the dashed bracket show that the equilibrium VAN concentration was lower than the detection limit of the UV-vis spectrophotometer, thus R~95% is likely underestimated.

FIG. 40 is a graph illustrating AHCNC-CHA-mediated VAN (C_{0,VAN}=1 mg mL⁻¹) removal percentage (R) at varying concentrations of maleic acid. AHCNC-CHA dose was 20 mg mL⁻¹, and total sample volume=1 mL. In the graph, MA stands for maleic acid. The data within the dashed bracket show that the equilibrium VAN concentration was lower than the detection limit of the UV-vis spectrophotometer, thus R~95% is likely underestimated.

FIG. 41 is a graph illustrating R at 20 mM of maleic acid (MA) and varying AHCNC-CHA doses. C_{0,VAN}=1 mg mL⁻¹. The data within the dashed bracket show that the equilibrium VAN concentration was lower than the detection limit of UV-vis spectrophotometer, thus R~95% is likely underestimated.

FIG. 42 is a graph illustrating AHCNC-CHA-mediated VAN (C_{0,VAN}=1 mg mL⁻¹) removal percentage (R) at varying concentrations of phosphatidylcholine (PC). AHCNC-CHA dose was 20 mg mL⁻¹, and total sample volume=1 mL. The data within the dashed bracket show that the equilibrium VAN concentration was lower than the detection limit of the UV-vis spectrophotometer, thus R~95% is likely underestimated.

FIG. 43 is a graph illustrating R at varying C_{0,VAN} in the FaSSIF or FeSSIF. AHCNC-CHA dose was 20 mg mL⁻¹, and total sample volume=1 mL.

FIG. 44 is a graph illustrating removal capacity (qₑ) at varying C_{0,VAN} in the FaSSIF or FeSSIF. AHCNC-CHA dose was 20 mg mL⁻¹, and total sample volume=1 mL. μ(VSEfm), cultured in the supernatant of VAN after incubation with AHCNC, CHA, or AHCNC-CHA for 4 h. The concentration of all sorbents (AHCNC, CHA, or AHCNC-CHA) was fixed at 20 mg mL⁻¹. CHA, AHCNC, and AHCNC-CHA control groups are VAN-free.

FIG. 46 is a graph illustrating VAN removal percentage (R) of AHCNC-CHA (20 mg mL⁻¹) at C_{0,VAN} below 100 μg mL⁻¹, determined by HPLC. The data within the dashed bracket shows that the equilibrium VAN concentration was lower than the detection limit of HPLC, thus R~75% is likely underestimated.

FIG. 47 is a graph illustrating the optical density (OD₆₀₀) of bacteria (VSEfm), cultured in the supernatant of VAN after incubation with AHCNC-CHA at varying periods. The concentration of all sorbents (AHCNC, CHA, or AHCNC-CHA) was fixed at 20 mg mL⁻¹. CHA, AHCNC, and AHCNC-CHA control groups are VAN-free.

FIG. 48 is a graph illustrating the optical density (OD₆₀₀) of bacteria (VSEfm), cultured in the supernatant of VAN after incubation with varying AHCNC-CHA doses for 4 h.

FIG. 49 is a graph illustrating optical density (OD₆₀₀) of bacteria (VSEfm), cultured in the supernatant of VAN incubated with varying acid (HCl, 5 M)-treated AHCNC-CHA doses for 4 h.

FIG. 50 is a graph illustrating the fecal density at each time point for total E. faecium.

FIG. 51 is a graph illustrating the fecal density at cumulative shedding for total E. faecium. Cumulative shedding is defined as the total area under the fecal density time-series curve (AUC) for the duration of the study. Data represent the mean±SEM, and significant differences were determined by the Kruskal-Wallis test, followed by the Dunn's post-hoc test for pairwise multiple comparisons (*P<0.05 and **P<0.01).

FIG. 52 is a schematic illustration of a timeline and protocol of the mouse model used to assess the anti-VAN capability of AHCNC-CHA in vivo.

FIG. 53 is a graph illustrating daily changes in body mass (% change relative to day 0) following the twice daily treatment with either 0, 4, or 10 mg AHCNC-CHA.

FIG. 54 is a graph illustrating the fecal density at each time point for VREfm.

FIG. 55 is a graph illustrating the fecal density at cumulative shedding for VREfm. Cumulative shedding is defined as the total area under the fecal density time-series curve (AUC) for the duration of the study. Data represent the mean±SEM, and significant differences were determined by the Kruskal-Wallis test, followed by the Dunn's post-hoc test for pairwise multiple comparisons (*P<0.05 and **P<0.01).

FIG. 56 is a graph illustrating the fecal density at each time point for VSEfm.

FIG. 57 is a graph illustrating the fecal density at cumulative shedding for VSEfm. Cumulative shedding is defined as the total area under the fecal density time-series curve (AUC) for the duration of the study. Data represent the mean±SEM, and significant differences were determined by the Kruskal-Wallis test, followed by the Dunn's post-hoc test for pairwise multiple comparisons (*P<0.05 and **P<0.01).

FIG. 58 is a graph illustrating the proportion of VREfm (of total E. faecium) at each time point.

FIG. 59 is a graph illustrating the cumulative proportion of VREfm (of total E. faecium) across all time points. Data represent the mean±SEM, and significant differences were determined by the Kruskal-Wallis test, followed by the Dunn's post-hoc test for pairwise multiple comparisons (*P<0.05 and **P<0.01).

FIG. 60 is a schematic illustration of an exemplary method for synthesizing an antibiotic removal agent, particularly a molecularly imprinted nanomaterial, for the removal of one or more antibiotic compounds.

FIG. 61 shows FE-SEM images of HCNC, zwitterionic poly(sulfobetaine methacrylate-co-2-aminoethyl methacrylate) (pSBAE), and VAN-imprinted polymerized zwitterionic hairy cellulose nanocrystals (ViPZ-HCNC). Scale bars are 500 nm.

FIG. 62 is a schematic illustration of ViPZ-HCNC, showing the formation of more cavities by increasing the VAN NH3+:HCNC COO⁻ molar ratio. Note that the HCNC scheme in the figure is only a representation of a small area on the HCNC hairs or crystalline body where PG and pSBAE polymerization occurs, which do not show the exact morphology of HCNC.

FIG. 63 is a graph showing R_{VAN}, R_{BSA}, R_{Lyz}, and R_{Lys} of ViPZ-HCNC compounds E, B, and D, prepared at varying VAN NH₃⁺:HCNC COO⁻ molar ratios.

FIG. 64 is a graph showing R_{VAN}, R_{BSA}, R_{Lyz}, and R_{Lys} of PG-coated HCNC (compound F) and ViPZ-HCNC compounds C and D, prepared at varying pSBAE concentrations.

FIG. 65 is a schematic illustration of ViPZ-HCNC, prepared at varying pSBAE concentrations and a constant VAN NH₃⁺:HCNC COO⁻ molar ratio. Note that the HCNC scheme in the figure is only a representation of a small area on the HCNC hairs or crystalline body where PG and pSBAE polymerization occurs, which do not show the exact morphology of HCNC.

FIG. 66 is a graph showing IF of ViPZ-HCNC compounds A, B, C, and D, prepared at varying VAN NH₃⁺:HCNC COO⁻ molar ratio and pSBAE concentrations.

FIG. 67 is a graph showing R_{VAN} of ViPZ-HCNC (compounds A, B, C, D) and pSBAE prepared at varying Ca²⁺ concentrations.

FIG. 68 is a graph showing R_{VAN} at varying ViPZ-HCNC doses and C_{0,VAN}=1 mg mL⁻¹ and incubation time=2 h.

FIG. 69 is a graph showing R_{VAN} at varying incubating times and a constant C_{0,VAN} (1 mg mL⁻¹), showing that incubation time for cavity-mediated VAN removal is about 2 h. The schematic illustrates how the ViPZ-HCNC cavities become progressively occupied by VAN molecules over time.

FIG. 70 is a graph showing R_{VAN} at varying C_{0,VAN}.

FIG. 71 is a graph showing qₑ versus C_{e,VAN}.

FIG. 72 is a graph showing R_{VAN} at varying C_{0,VAN} in SIFs.

FIG. 73 is a graph showing VAN qₑ at varying C_{0,VAN} in SIFs.

FIG. 74 shows fluorescence microscopy images of NIH/3T3 murine fibroblast cells, cultured in the media, containing varying ViPZ-HCNC concentrations, on day 7. Live and dead cells are labeled in green and red, respectively. The cells are incubated in ViPZ-HCNC-free media as a control group. The scale bar is 500 μm.

FIG. 75 is a graph showing the viability of NIH/3T3 cells, treated with varying ViPZ-HCNC concentrations, on day 7.

FIG. 76 is a graph showing the metabolic activity of NIH/3T3 fibroblast cells, cultured for 7 days in the media, supplemented with varying concentrations of ViPZ-HCNC, quantified using the PrestoBlue™ cell viability reagent.

### DETAILED DESCRIPTION OF THE INVENTION

The following description is of exemplary embodiments presently contemplated for carrying out the present invention. This description is not to be taken in a limiting sense but is made merely for the purpose of describing the general principles and features of the present invention. The scope of the present invention should be determined with reference to the claims.

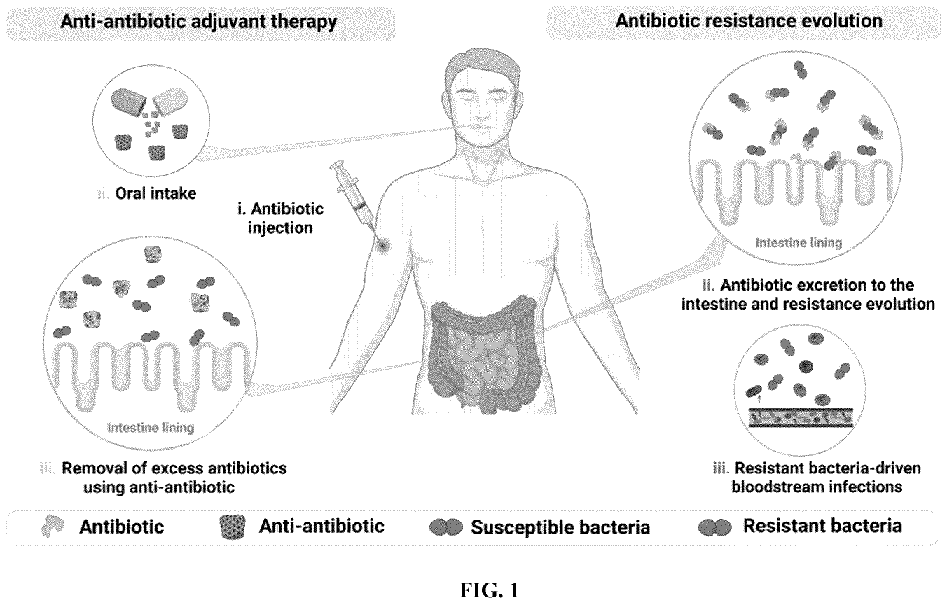

FIG. 1 provides an illustration of an exemplary embodiment of a process for the removal of one or more antibiotic compounds within a patient's GI tract while that patient is undergoing one or more intravenous (IV) antibiotic treatments via one or more intravenous injections. Exemplary antibiotic compounds may include, but not be limited to, B-lactams, aminoglycosides, chloramphenicol, glycopeptides, quinolones, oxazolidinones, sulfonamides, tetracyclines, macrolides, ansamycins, streptogramins, lipopeptides, or combinations thereof. An exemplary antibiotic compound may include, but not be limited to, vancomycin (VAN). In some embodiments, the IV antibiotic treatment can include VAN alone or VAN in combination with one or more other antibacterial compounds (e.g. linezolid, daptomycin (DAP), etc.). In other embodiments, the IV antibiotic treatment can include DAP and/or other antibacterial compounds (e.g. linezolid, etc.).

A patient can orally take one or more capsules, pills, or other type of orally configured receptacle that can include an antibiotic removal agent or an antibiotic deactivation agent. The orally taken antibiotic removal agent or antibiotic deactivation agent can be taken as a powder that is swallowed via the mouth of the patient in powder form, such as after the powder is mixed into a liquid (e.g. water) for the patient to drink. The orally digestible antibiotic removal agent or antibiotic deactivation agent can also be in a pill form, be in a pill form that is coated with a coating, or be in some other suitable form that can be digested directly (e.g. without being coated or positioned within a capsule or other type of oral delivery mechanism) for digestion of the agent(s) by the patient for the agent(s) being delivered to the patient GI tract via its oral digestion.

As used herein, the term “patient” may refer to any biological system to which an antibiotic removal agent or an antibiotic deactivation agent can be administered, including without limitation, humans, other animals, (e.g., dogs, cats, horses, cows, pigs, chickens, cattle, etc.), pets, etc.

The oral digestion of the antibiotic removal agent or antibiotic deactivation agent can occur during the IV antibiotic treatment, before the IV antibiotic treatment, or shortly after the IV antibiotic treatment in different embodiments. The oral digestion of the antibiotic removal agent or antibiotic deactivation agent can alternatively occur during or shortly after an antibiotic compound is taken by the patient in any other type of antibiotic administration or treatment protocol (e.g. taken orally by the patient as a pill, etc.).

For example, in some embodiments, the oral digestion of the antibiotic removal agent or antibiotic deactivation agent can be timed to occur so that the antibiotic removal agent or antibiotic deactivation agent can be digested and subsequently be present within the GI tract of a patient so that after an IV treatment of one or more antibiotic compounds, the antibiotic removal agent or antibiotic deactivation agent is present in the GI tract for removal or deactivation of any of the one or more antibiotic compounds from the patient GI tract so that any antibiotic compound(s) that may pass into the patient's GI tract after being injected into the patient can be effectively removed from the GI tract or effectively deactivated from the GI tract (e.g. adsorbed, absorbed, or otherwise biologically inserted via interaction between the antibiotic removal agent or antibiotic deactivation agent and the antibiotic compound(s)).

In some embodiments, the patient can be on an oral digestion protocol for repeatedly taking the antibiotic removal agent or antibiotic deactivation agent over the course of a period of time (e.g., every 2 hours, every 4-6 hours, every 4-12 hours, etc.) during the time the patient may undergo the IV treatment of the antibiotic compound(s).

In other embodiments, the oral digestion may be timed to occur before, during or after an antibiotic injection or antibiotic treatment is administered.

As can be appreciated from the left side of the schematic illustration of FIG. 1, the antibiotic removal agent or antibiotic deactivation agent can be configured to react with excess antibiotic compounds (e.g., VAN) present in the patient GI tract after the patient is injected with the antibiotic compound(s). The removal can occur via the antibiotic compounds being adsorbed into the antibiotic removal agent or antibiotic deactivation agent due to an affinity the antibiotic removal agent can have for the antibiotic compound(s). The removal may also occur by a chemical reaction or other type of interaction with the antibiotic removal agent or antibiotic deactivation agent and the antibiotic compound(s) in the GI tract that reduces or eliminates the bioreactivity of the antibiotic compounds so that bacteria within the IG tract are unable to react to the antibiotic compound and are not affected by that compound after it reacts with the antibiotic removal agent or antibiotic deactivation agent in the patient GI tract.

As can be appreciated from a comparison of the right side of FIG. 1 with the left side of FIG. 1, the use of the antibiotic removal agent or antibiotic deactivation agent can avoid antibiotic compounds being bioreactive in the patient's GI tract so that those compounds do not kill healthy gut bacteria of the patient and also avoid antibiotic resistance evolution by causing such antibiotic resistant bacteria to be produced in an increasing proportion in the patient's GI tract while that patient undergoes an IV antibiotic treatment or other type of antibiotic treatment.

FIG. 12 provides an illustration of an exemplary antibiotic removal agent or antibiotic deactivation agent. In exemplary embodiments, the antibiotic removal agent or antibiotic deactivation agent includes hairy cellulose nanocrystals (HCNC) hybridized with, or immobilized on, a resin. In other embodiments, the antibiotic removal agent or antibiotic deactivation agent includes functionalized biopolymers. In still other embodiments, the antibiotic removal agent or antibiotic deactivation agent includes hairy cellulose nanocrystals (HCNC) hybridized with, or immobilized on, a resin, in combination with functionalized biopolymers.

The HCNC may include cellulose (polysaccharide) bodies, such as cellulose fibrils and/or crystals. In some embodiments, the cellulose bodies may be nanoscale bodies. For example, the cellulose bodies may be nanofibrils and/or nanocrystals. The cellulose bodies may be derived from any suitable source. In some embodiments, the cellulose bodies may be biosourced (e.g., derived from biomass). For example, the cellulose bodies may be plant-based, such as derived from wood pulp, cotton, algae, or any other suitable plant-base source. In some embodiments, the cellulose bodies may be non-toxic and and/or biodegradable.

The cellulose bodies may have a plurality of cellulose chains (also referred to as “hairs”) protruding therefrom, such as protruding from the ends of the bodies. The cellulose chains may be functionalized with anionic groups. The anionic groups may be selected from the group consisting of carboxylates, dicarboxylates, sulfates, or any other suitable anionic group. The anionic groups may serve as active sites configured to adsorb to the antibiotic compound(s).

In some embodiments, the cellulose chains are functionalized with cationic groups. In some embodiments, the cellulose chains are functionalized with amphiphilic groups. In some embodiments, the cellulose chains are functionalized with zwitterionic groups. In some embodiments, the cellulose chains are functionalized with a combination of electrically neutral anionic, cationic, zwitterionic, and/or amphiphilic groups.

The cellulose bodies may be any shape and/or size, though it is contemplated that the bodies may be rod-like or needle-like structures. Advantageously, the cellulose bodies are elongated in shape; that is, having a length/width ratio higher than 1.

The HCNC may be hybridized with, or immobilized on, a resin. The resin may be in particulate form, such as in microparticulate form. In particular, the HCNC may be adsorbed to the surface of the particulate resin and/or within the pores of the particulate resin. The resin may therefore advantageously be porous.

In some embodiments, the particulate resin may include cholestyramine (CHA), patiromer, sodium polystyrene sulfonate, sodium zirconium cyclosilicate, sodium polystyrene sulfonate, sodium polystyrene sulfonate, colestipol, tolevamer, polacrilex resin (Amberlite IRP64), polacrilin potassium (Amberlite IRP88), colesevelam, sevelamer (SEV), magnesium-containing compounds, calcium-containing compounds, iron-containing compounds, zinc-containing compounds, antacids containing aluminum or magnesium, proton pump inhibitors, or combinations thereof.

In some embodiments, the resin includes or consists of cholestyramine (CHA). In such embodiments, the antibiotic removal agent or antibiotic deactivation agent may be designated as an HCNC-CHA agent.

The functionalized biopolymers may include polysaccharide, protein, polynucleotides, and/or polypeptides, wherein the biopolymer is functionalized with a combination of electrically neutral anionic, cationic, zwitterionic, and/or amphiphilic groups.

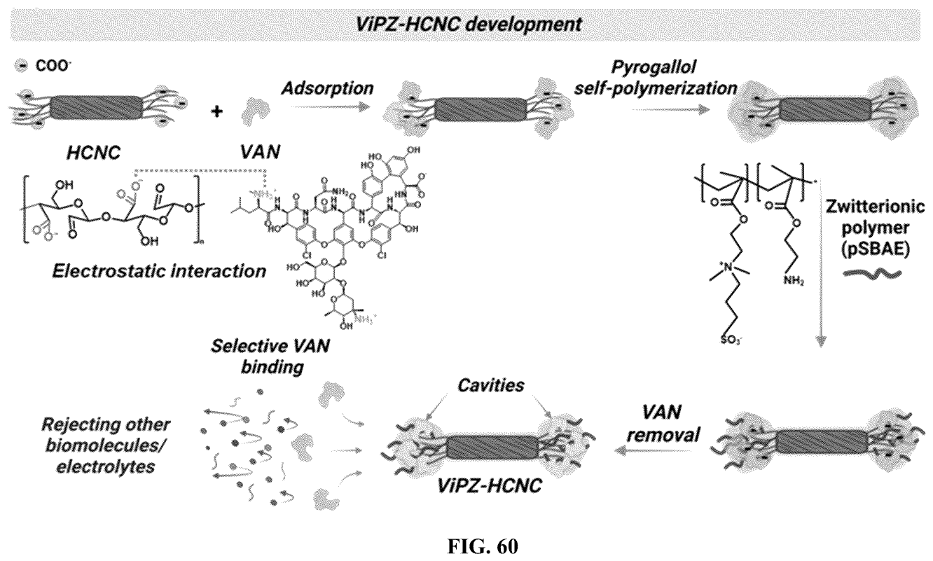

FIG. 60 provides an illustration of an exemplary embodiment of a method for synthesizing an antibiotic removal agent, particularly a molecularly imprinted nanomaterial, for the removal of one or more antibiotic compounds. The nanomaterials may be employed to enable highly selective recognition and removal of an antibiotic compound, such as VAN, with minimized nonspecific adsorption of other biomolecules.

The nanomaterials may include HCNC, as described in detail above. The HCNC may be functionalized with specific recognition sites through a molecular imprinting process utilizing the target antibiotic compound as the template molecule.

Alternatively, the nanomaterials may include functionalized biopolymers, as described in detail above, which may similarly be functionalized with specific recognition sites through a molecular imprinting process.

Referring to FIG. 60, a method of forming the molecularly imprinted nanomaterial includes a step of adsorbing the target antibiotic compound onto the HCNC or functionalized biopolymer via electrostatic interactions. For example, the positively charged antibiotic molecules can interact with negatively charged functional groups, such as carboxylates, present on the HCNC or biopolymer surface.

The method further includes a step of polymerizing a zwitterionic polymer around the antibiotic-adsorbed HCNC or biopolymer. This forms a coating that can impart antifouling and hydration properties to the material. The polymerization can be mediated via a crosslinking agent, which can facilitate covalent attachment of the zwitterionic polymer to the surface, thereby creating a stable, imprinted shell with specific cavities that are complementary in shape and functional groups to target antibiotic compound.

The zwitterionic polymer is not particularly limited. In some embodiments, the zwitterionic polymer is poly(sulfobetaine) (pSBAE).

The crosslinking agent is similarly not particularly limited, and in some embodiments can be purpurogallin (PG).

The method further includes a step of removing the template antibiotic molecules using a suitable surfactant or solvent. The surfactant or solvent dissolves or detaches the target antibiotic compound and leaves behind HCNC or biopolymers configured with cavities that are shape- and functionally-specific for antibiotic recognition.

The resulting nanostructure may exhibit high binding affinity and selectivity for the target antibiotic compound. The zwitterionic coating effectively minimizes nonspecific adsorption of proteins or other biomolecules, owing to the formation of a hydration layer that reduces fouling, while enabling the specific cavities to recognize and bind antibiotic molecules with high selectivity.

It is understood that the molecularly imprinted nanomaterials, may be capable of operating effectively within complex media, including intestinal fluids containing electrolytes, bile acids, and other biomolecules.

Ultimately, as can be appreciated from the above, embodiments generally relate to processes and apparatus configured to reduce, hinder, or avoid the growth or development of drug resistant bacteria and/or the protection of drug sensitive bacteria.

Embodiments can additionally, or alternatively, be adapted to help block or avoid antibiotic-resistant bacterial growth within the body of a patient, for example. Embodiments can additionally, or alternatively, be adapted to help protect or promote antibiotic-sensitive bacterial growth within the body of a patient, for example. Embodiments can additionally, or alternatively, be adapted to help protect the microbiome within the body of a patient, for example.

FIG. 12 provides an illustration of an exemplary antibiotic removal agent or antibiotic deactivation agent that is structured in a particulate form that has pores that adsorb and/or absorb the antibiotic compounds within the GI tract, for example. The antibiotic removal agent or antibiotic deactivation agent can render the antibiotic compounds inert to the GI bacteria and can be passed out of the patient via the patient's feces or urine without having any significant bioreactivity for bacteria due to the functionality of the antibiotic removal agent particulate or the antibiotic deactivation agent particulate. The antibiotic removal agent or antibiotic deactivation agent consumed by the patient may then be passed out of the patient via urine or feces so the antibiotic compound(s) are inert (e.g. non-bioreactive) to bacteria as well.

Embodiments of the process can be performed in a hospital or other type of care facility (e.g., doctor office, urgent care facility, etc.). It is also contemplated that embodiments can be performed in a farm setting to treat farm animals to help prevent the evolution of antimicrobial resistant bacteria in farm animals (e.g., cattle, cows, chickens, pigs, turkeys, poultry, fish, etc.). Embodiments of the process can also be employed to facilitate administering of one or more antibiotic removal agents or one or more antibiotic deactivation agents at different times each day while a patient is undergoing IV injections of one or more antibiotics for treatment of an infection that is not located in the patient's GI tract.

In some embodiments, the one or more antibiotic compounds may be used to treat an infectious disease in a patient. The infectious disease may be located in the GI tract, or may not be located in the GI tract. In other embodiments, the one or more antibiotic compounds may be used to treat a non-infectious disease in a patient.

The non-infectious disease may be located in the GI tract, or may not be located in the GI tract.

As discussed further herein, we have found that some embodiments of our process can be utilized in conjunction with VAN so that VAN can be removed from the GI tract and/or have VAN's bioreactivity with bacteria removed so that the removed VAN does not affect the patient's GI tract bacteria (e.g., it can avoid the GI tract bacteria from dying due to the antibacterial nature of the VAN after adsorption and/or absorption by the antibiotic removal agent). For example, the agents, when ingested by the patient, can function to block the activity of VAN (or other antibiotic compound(s)) at physiologically relevant concentrations to effectively remove the antibiotic compounds from the patient's GI tract so that the patient's gut bacteria does not react to those compounds (e.g. does not die, does not die in a way that results in driving the uptake of resistant bacteria strains in evolution of the gut bacteria, etc.). This removal can occur via adsorption, diffusion, and/or absorption of the antibiotic compound(s) in the patient GI tract via the antibiotic removal agent having or being one or more antibacterial agent removal compounds or antibacterial agent deactivation compounds in some embodiments.

For example, as discussed below, we conducted HCNC-CHA agent VAN removal via conducting in vitro experiments at controlled initial antibiotic concentrations, pH, and ionic strengths. The mechanisms of antibiotic removal by HCNC-CHA were also examined under the influence of individual intestinal fluid components, e.g., bile acid (BA), maleic acid (MA), phosphatidylcholine (PC), and the whole simulated intestinal fluid (SIF).

### EXAMPLES

### Example 1

### Methods and Materials

In our conducted evaluation and experimentation work, Northern bleached softwood sheets (Domtar Inc., Canada) served as the starting cellulose material. Sodium (meta)-periodate (NaIO₄, >99.0%), sodium chloride (NaCl, >99.5%), sodium chlorite (NaClO₂, 80%), hydrogen peroxide (H₂O₂, 30 wt %), sodium hydroxide (NaOH, ACS Reagent, >97%), hydrochloric acid (HCl, ACS reagent, 37%), ethylene glycol (Reagent plus >99%), poly-L-lysine (PLL, 0.1% w/v), hydroxylamine hydrochloride (NH₂OH·HCl, Reagent plus 99%), calcium chloride dihydrate (CaCl₂·2H₂O, for molecular biology, ≥99.0%), centrifuge tube filter cellulose acetate membrane, 0.22 μm pore size), L-α-phosphatidylcholine (type XVI-E, ≥99.0%), and cholestyramine resin (CHA, Dowex® 1×2 chloride form) were purchased from MilliporeSigma (USA). BA (C₂₆H₄₄NNaO₇S) and MA (C₄H₄O₄, >98%) were purchased from Spectrum Chemical and Beantown Chemical (USA), respectively. Mica sheets (V1 grade) and Krazy glue were purchased from Ted Pella Inc. (USA) and Agar Scientific (UK), respectively. Fasted state simulated intestinal fluid (FaSSIF-V2) and fasted state simulated gastric fluid (FaSSGF) in the powder form were procured from Biorelevant (UK). Vancomycin hydrochloride (USP grade, >95%) was purchased from VWR International (USA). Anhydrous ethanol (200 proof) was supplied by KOPTEC (USA). Conductive double-sided adhesive carbon tapes were purchased from Rave Scientific (USA). Formic acid (CH₂O₂, >99%), Optima™ water (liquid chromatography-mass spectroscopy (LC-MS) grade), and acetonitrile (Honeywell Burdick & Jackson Brand™, C₂H₃N, >99.9%) were procured from Thermo Fisher Scientific (USA). Milli-Q water (resistivity=18.2 mΩ cm at 25° C.) was generated from deionized (DI) water using a remote water purification system (Direct-Q 5, Millipore, USA).

The bacterial culture media, Mueller Hinton II Broth (cation-adjusted), Enterococcosel Agar (BBL formulation), and Brain Heart Infusion Broth (BHI, BBL formulation were purchased from BD Difco (USA). Two E. faecium clones, BL00239-1, and BL00239-1S, were derived from a clinical isolate collected from a patient undergoing treatment for a VREfm bloodstream infection at the University of Michigan hospital. Phosphate-buffered saline (PBS) solution (1×) was purchased from Thermo Fisher Scientific (USA). Glycerol was obtained from VWR International (Molecular biology grade, USA). Sterile 0.9% saline solution was supplied from Aspen Veterinary Solutions (USA). Eight-week-old female Swiss-Webster mice were purchased from Charles River (USA), and 5053-PicoLab Rodent Diet 20 was supplied by LabDiet (USA). Veterinary grade VAN for mouse injection and ampicillin were obtained from Slate Run Pharmaceuticals (USA) and Alfa-Aesar, respectively.

AHCNC synthesis: Pulp sheets (1 g) torn into ~2×2 cm² thin pieces were soaked in DI water. The wet pulp disintegrated via mechanical stirring overnight and then vacuum-filtered. Afterwards, NaCl (3.8 g) and NaIO₄ (1.32 g) were dissolved in DI water (total volume=65 mL, including the remaining water in the wet pulp) in an aluminum foil-wrapped beaker to prevent light-triggered NaIO₄ deactivation, followed by the addition of wet pulp and stirring at ambient temperature for 42 h.

To stop the oxidation reaction by quenching the unreacted NaIO₄, 1 mL of ethylene glycol was added to the mixture. Partially oxidized fibrils, called dialdehyde-modified cellulose (DAMC), were rinsed at least 5 times with DI water, followed by vacuum filtration. Never-dried DAMC was then oxidized via reacting with NaClO₂ (0.8452 g) and H₂O₂ (0.8478 mL) in DI water (50 mL). NaOH (0.5 M) was intermittently added during reaction while stirring the mixture for 12 h at room temperature to maintain the pH at 5. After completion, the mixture was centrifuged at 27000×g for 15 min to eliminate any residual unfibrillated fibrils. The AHCNC was separated from the collected supernatant via precipitation using ethanol as a non-solvent (0.16 g per 1 g of dispersion). The milky dispersion was then centrifuged at 3000×g for 15 min. Finally, excess ethanol was added to the remaining supernatant to precipitate fully solubilized dicarboxylated cellulose (DCC) polymers, separated by centrifugation at 3000×g. The AHCNC and DCC were redispersed separately in DI water (~50 mL) and dialyzed (Spectra/Por dialysis bags, molecular weight cutoff=6-8 kDa) against DI water for 3 days, followed by concentration measurement via oven drying and weighing 1 mL of dispersion.

AHCNC-CHA hybridization: AHCNC-CHA biomaterials were prepared by incubating CHA microparticles (20 mg) with varying concentrations of AHCNC dispersions (total volume=2 mL) for 24 h at ambient temperature. The mixture was then centrifugated at 15000×g for 5 min, the supernatant was removed, and Milli-Q water (1 mL) was used to rinse the samples at least 3 times to remove any unbound AHCNC. The quantity of AHCNC immobilized onto CHA was measured by subtracting the dry mass of free AHCNC in the supernatant from the total mass of initial AHCNC. The AHCNC-CHA was then dried in the oven at 37° C. overnight for further experiments.

Aldehyde group content measurement: The aldehyde group content of DAMC, the intermediate product for AHCNC synthesis, was quantified using an oxime titration method. A certain amount of wet DAMC fibrils was suspended in Milli-Q water (50 mL), and the pH was adjusted to 3.5 by adding HCl (0.1 M), followed by adding a NH₂OH·HCl solution (5 wt %, 10 mL) with the same pH (3.5, adjusted using a 0.1 M NaOH solution) and stirring. The reaction of aldehyde groups with NH₂OH·HCl (oxime reaction) yielded HCl. The pH decrease was compensated via gradually adding a NaOH solution (10 mM, 40 mL) at a rate of 0.1 mL min-1 using an automatic titrator (Metrohm 907 Titrando, USA). The amount of NaOH consumed to reach the initial pH (i.e., 3.5) was used to calculate the DAMC aldehyde content.

Carboxylate group content measurement: Carboxylate group content of AHCNC before and after immobilization onto the CHA resin was quantified by conductometric titration. A certain amount of material (20 mg) was dispersed/suspended in Milli-Q water (140 mL), including 2 mL of NaCl solution (20 mM). Then, pH was reduced to 3 via adding a HCl solution (stock solution concentration=0.1 M). Using the automatic titrator, the mixture was titrated with a NaOH solution (10 mM) at a rate of 0.1 mL min-1 until the pH reached ~11. The content of carboxyl groups was obtained based on the middle portion of titration curves, associated with the weak acid.

Quantification of adsorbed AHCNC in the AHCNC-CHA: To quantify adsorbed AHCNC in AHCNC-CHA, the suspension of AHCNC-CHA was centrifugated at 15000×g for 5 min after hybridization. Then, the supernatant (1 mL) was dried at 37° C. in an oven overnight to measure the free (unadsorbed) AHCNC mass (m_{AHCNC}). The AHCNC adsorption ratio (m_{A,AHCNC}/m_{CHA}) was then calculated using Equation (1):

$$AHCNC adsorption ratio = \frac{{m}_{0 , AHCNC} - {m}_{AHCNC}}{{m}_{CHA}} \times 100 \% \qquad ( 1 )$$

where m_{0,AHCNC} represents the initial mass of AHCNC before adding to the CHA suspension, and m_{CHA} (mg) denotes CHA mass in the AHCNC-CHA biomaterial.

To quantify the desorbed AHCNC from AHCNC-CHA, the AHCNC-CHA suspension was incubated at varying pH for 6 h, followed by centrifugation at 15000×g for 5 min. Then, the supernatants were dried at 37° C. in an oven overnight to calculate the desorbed AHCNC mass (m_{D,AHCNC}). The AHCNC desorption ratio (m_{D,AHCNC}/m_{AHCNC-CHA}) was then measured via dividing m_{D,AHCNC} by the initial mass of AHCNC-CHA (m_{AHCNC-CHA}) incubated at varying pH.

Atomic force microscopy (AFM) imaging: To investigate the morphology of AHCNC, a multimode AFM (Bruker Dimension Icon, USA) was used in the PeakForce tapping mode using a silicon nitride probe (Bruker ScanAsyst-Air, USA). To prepare the AHCNC sample for imaging, freshly cleaved mica was adhered to a stainless-steel disc using the Krazy glue and pre-coated with a PLL solution (10 μL, 0.1% w/v). After 10 min, the excess PLL was rinsed with Milli-Q-water. Then, 10 μL of AHCNC dispersion (0.1 mg mL⁻¹) was placed on the mica sheet, air-dried overnight, rinsed gently 5 time with 100 μL of Milli-Q water, and air-dried overnight again before AFM imaging. The AFM images were analyzed using the NanoScope Analysis software (Version 1.80, accessed via Penn State Materials Characterization Laboratory, MCL). Length and width of >50 particles per image (n=3) were measured by the Gwyddion software (Version 2.49, accessed via Penn State MCL).

Hydrodynamic size and ζ-potential assessments: The hydrodynamic equivalent size of AHCNC, VAN, and AHCNC-VAN was measured at 25° C. and 90° scattering angle using a dynamic light scattering (DLS) instrument (Malvern Zetasizer Nano series, UK). Before conducting the measurements, the samples were diluted to 0.1% w/v using ultrapure water at desired pH (adjusted using a 2M NaOH or 1M HCl solutions), Na⁺ concentration, and/or Ca²⁺ concentrations. Additionally, the hydrodynamic size of AHCNC (0.1% w/v) was measured at varying initial VAN concentrations (0-10 mg mL⁻¹, pH=6.5, adjusted using 0.5 M NaOH). Subsequently, the samples (70 μL) were pipetted into low-volume quartz cuvettes (ZEN2112, Malvern, UK) to conduct the spectroscopy. The Z-average (cumulants mean) of intensity measurements were reported as the hydrodynamic equivalent size of particles.

The ζ-potential of AHCNC, VAN, and AHCNC-VAN was estimated based on the electrophoretic mobility, measured by the Nano ZS Zetasizer (Malvern instrument, UK). Before measuring ζ-potential, samples were diluted to 0.1% w/v using ultrapure water at desired pH (adjusted using a 2 M NaOH or 1 M HCl solutions), Na⁺ concentration, and/or Ca²⁺ concentrations. Additionally, the ζ-potential of AHCNC (0.1% w/v) was measured at varying initial VAN concentrations (0-10 mg mL⁻¹, pH=6.5, adjusted using 0.5 M NaOH). Samples (900 μL) were then pipetted into the universal dip cell kit (Malvern, UK) for conducting the measurements. The particles have an average width of ~7 nm, and ƒ(κa)~0.5 (K and a denote the Debye-Hückel parameter and the particle radius, respectively). Accordingly, the ζ-potential of particles was estimated by applying Equation (2) to the electrophoretic mobility.

$${μ}_{av} = \frac{{μ}_{‖} + 2 {μ}_{⊥}}{3} = \frac{{ε}_{r} {ε}_{0}}{3 η} ζ [ 1 + 2 f ( κ a ) ] \qquad ( 2 )$$

where μ_{∥} and μ_{⊥} denote the electrophoretic mobility for cylindrical particles in the parallel and perpendicular direction to the electric field, respectively, Er is the relative dielectric permittivity of solvent, ε₀ denotes the dielectric permittivity in a vacuum i.e., vacuum permittivity, and η is the viscosity. For VAN, ζ-potential was estimated using the Smoluchowski equation.

Attenuated total reflectance (ATR)-Fourier transform infrared (FTIR) spectroscopy: The functional groups of AHCNC, CHA, and AHCNC-CHA were characterized using a Vertex 70 FTIR spectrometer (Bruker, USA) with a Harrick Diamax ATR accessory following the principles of the Bouguer-Beer-Lambert law. The samples were dried at 37° C. for two days to ensure water removal, eliminating the water absorption peaks. The dried samples were placed onto the ATR crystal, and the tip of pressure clamp was gently lowered to apply maximum pressure. The ATR-FTIR spectra were obtained by averaging a total of 100 scans in a wavenumber range of 500-4000 cm⁻¹ (resolution=6 cm⁻¹). The spectrum of the bare diamond was used as a reference to establish a baseline and normalize the absorbance values.

Scanning electron microscopy (SEM) imaging: Dried CHA or AHCNC-CHA were secured to sample pins using a double-sided adhesive carbon tape and coated with a 3 nm-thick indium layer using a vacuum sputtering instrument (Leica sputter coating EM ACE 600, USA). The particles were then imaged under SEM (Thermo Scientific Verios G4, USA) with an accelerating voltage of 3.0 kV and a beam current of 0.1 nA.

Surface area and pore size analyses: Nitrogen adsorption and desorption isotherm measurements were conducted on a Micromeritics instrument (ASAP™ 2420, Micromeritics Instrument, GA, USA) at 77 K. CHA and AHCNC-CHA samples were degassed under vacuum for 4 h prior to measurements to remove any unwanted vapors and gases, and then transferred to analysis ports. The specific surface area was determined using Brunauer-Emmet-Teller (BET) equation. Additionally, pore volume and pore diameter were obtained using the Barrett-Joyner-Hollande (BJH) model. The calculations were performed using the ASAP™ 2420 software (package V6.07).

In vitro VAN removal: In all the removal experiments, three steps were followed: (i) VAN stock solutions (concentration=40 mg mL⁻¹) were prepared by dissolving 800 mg of VAN in 20 mL of Milli-Q water. To prepare the VAN solutions with desired concentrations, the stock solution was further diluted using Milli-Q water. (ii) standard calibration lines were generated for each experimental condition by recording the absorbance of known concentrations of VAN solutions, ranging from 0.05 mg mL⁻¹ to 10 mg mL⁻¹ at the maximum peak of wavelength (λₘₐₓ)=280 nm. A total of 12 calibration lines were used to calculate VAN concentration in the supernatants, analyzed by an UV-vis spectrophotometry (Tecan Model Infinite 200 Pro, USA). (iii) The VAN removal percentage (R, %) and the equilibrium VAN removal capacity (qₑ, mg g⁻¹) were calculated according to Equations (3) and (4), respectively:

$$R \% = \frac{( {c}_{0} - {c}_{e} )}{{c}_{0}} \times 100 \qquad ( 3 )$$

$${q}_{e} = \frac{( {c}_{0} - {c}_{e} )}{m} \times V \qquad ( 4 )$$

where C₀ (mg mL⁻¹) and Cₑ (mg mL⁻¹) are the initial and equilibrium VAN concentrations, respectively, m (g) is the mass of sorbent material, and V (mL) is the total volume of solution. Note that the detection limit of VAN concentration by UV-vis spectrophotometry was ~0.05 mg mL⁻¹. The measured equilibrium VAN concentrations (i.e. Cₑ) lower than the detection limit were all assumed as 0.05 mg mL⁻¹. Therefore, the R values calculated via applying this assumption are all likely to be underestimated.

Effect of initial VAN concentration on VAN removal: The capability of AHCNC or AHCNC-CHA in removing VAN was investigated via batch removal experiments. VAN solutions were prepared by diluting the VAN stock solution with Milli-Q water to obtain varying concentrations (1-20 mg mL⁻¹). The pH of all solutions was adjusted to 6.5 by the addition of NaOH solution (0.5 M) and Milli-Q water to a total desired volume of 1 mL. The AHCNC (final concentration=0.25 mg) or AHCNC-CHA (20 mg) were added to the VAN solutions (final volume=1 mL), vortexed for 1 min, and the vials were maintained on the nutating mixer (Fisherbrand, USA) to shake at 60 rpm at room temperature. Agitation time varied depending on the experiment type. After centrifugation at 5000×g for 5 min, supernatants were collected, and the VAN concentration was determined using the UV-vis spectrophotometer at λₘₐₓ of 280 nm.

Effect of contact time on VAN removal: To investigate the effect of contact time on AHCNC or AHCNC-CHA-mediated VAN removal, VAN solutions with a constant concentration (1 mg mL⁻¹, total volume=1 mL, including the volume of acid or base used for pH adjustment to 6.5) were prepared by diluting VAN stock solutions with Milli-Q-water. The pH of all solutions was increased to 6.5 by adding a NaOH solution (0.5 M). AHCNC-CHA (20 mg) was then added to the VAN solutions, vortexed for 1 min, and the vials were placed on the nutating mixer, set to 60 rpm. At specific time intervals (5 min, 1, 2, 4, and 24 h), the samples were centrifuged at 5000×g for 5 min. Then, the supernatants were separated, and the VAN concentration was analyzed using the UV-vis spectrophotometer at λₘₐₓ=280 nm.

Effect of AHCNC-CHA dose on VAN removal: To examine the effect of AHCNC-CHA dose on VAN removal, VAN solutions with a concentration of 1 mg mL⁻¹ were prepared by diluting the VAN stock solution and adjusting the pH to 6.5 using a NaOH solution (0.5 M). Varying concentrations of AHCNC-CHA were then added to the VAN solutions, followed by vortexing for 1 min and maintaining the vials on the nutating mixer to agitate at 60 rpm. After 4 h of incubation, supernatants were separated by centrifugation at 5000×g for 5 min and analyzed by a UV-vis spectrophotometer at λₘₐₓ=280 nm.

Effect of pH on VAN removal: To investigate the ionization effects of AHCNC-CHA and VAN functional groups on VAN removal, the experiments were performed in VAN solutions at pH ranging from 2 to 12. VAN solutions with a constant concentration (1 mg mL⁻¹) were prepared by diluting the VAN stock solution in vials and adjusting the pH by adding a NaOH or HCl solution and Milli-Q water to a total solution volume of 1 mL. Then, AHCNC-CHA (20 mg) was added to the VAN solutions with varying pH, followed by vortexing for 1 min and maintaining the vials on the nutating mixer at 60 rpm. After 4 h of incubation, supernatants were separated by centrifugation at 5000×g for 5 min and analyzed by a UV-vis spectrophotometer at λₘₐₓ=280 nm.

Effect of ionic strength and ion type on VAN removal: To explore the effect of ion types and ionic strength on AHCNC-CHA-mediated VAN removal, the removal experiments were conducted at varying Na⁺ or Ca²⁺ concentrations ranging from 0 to 200 mM. VAN solutions with a concentration of 1 mg mL⁻¹ were prepared by diluting the VAN stock solution with Milli-Q water and adjusting the pH to 6.5 using a NaOH solution (0.5 M). AHCNC-CHA (20 mg) was then added to the VAN solutions containing varying Na⁺ or Ca²⁺ concentrations and vortexed for 1 min. The vials were maintained on the nutating mixer while agitating at 60 rpm. After 4 h of incubation, supernatants were separated by centrifugation at 5000×g for 5 min and analyzed using the UV-vis spectrophotometer at λₘₐₓ=280 nm.

Preparation of simulated intestinal fluid (SIF) and simulated gastric fluid (SGF) media: To simulate SIF in vitro, fasted state SIF (FaSSIF) and fed state SIF (FeSSIF) were formulated according to the standard protocols. For the FaSSIF, NaOH pellets (1.392 g), MA (2.22 g), and NaCl (4.01 g) were first dissolved in Milli-Q water (0.9 L). The pH of the solution was raised to 6.5 by adding a NaOH solution (0.1 M), and the total volume was adjusted to 1 L using Milli-Q water. Then, FaSSIF-V2 powder (1.79 g) was added to the solution and allowed to stir for 1 h at the ambient temperature. For the FeSSIF, sodium taurocholate (8.25 g) was added to the FaSSIF (250 mL) and stirred at the ambient temperature to fully dissolve. Subsequently, PC (2.95 g) was added to the solution and the final volume was adjusted to 1 L using FaSSIF. Finally, the mixture was stirred for an additional 4 h until it became clear.

To prepare the SGF solution, NaCl (1.999 g) and HCl (29.1 g, 1 M) were dissolved in Milli-Q water (0.9 L). The pH of solution was maintained at 1.6 using a HCl solution (1 M), and the total volume was adjusted to 1 L using Milli-Q water. Then, FaSSGF powder (0.06 g) was added to the solution and stirred for 1 h at the ambient temperature.

Effect of AHCNC-CHA pre-incubation time in SGF on VAN removal: AHCNC-CHA (100 mg) was first incubated in the SGF (5 mL) for varying periods (1-6 h) at 37° C. to mimic the food retention in the stomach. Afterwards, the suspensions were centrifugated at 15000×g for 5 min, the supernatants were decanted, and Milli-Q water was used to wash away any remaining SGF. The precipitates, SGF-incubated AHCNC-CHA, were then oven-dried at 37° C. overnight. To determine the effect of SGF incubation time on AHCNC-CHA-mediated VAN removal, SGF-incubated AHCNC-CHA (20 mg) were added to the VAN solutions (final concentration=1 mg mL⁻¹, pH=6.5, total volume=1 mL). The vials were then placed on the nutating mixer, agitating at 60 rpm. After 4 h of incubation, supernatants were separated by centrifugation at 5000×g for 5 min and analyzed by the UV-vis spectrophotometer at λₘₐₓ=280 nm.

Effect of SIF components on VAN removal: To examine the effect of SIF components on VAN removal, AHCNC-CHA (20 mg) was added to the VAN solutions (final concentration=1 mg mL⁻¹, total volume=1 mL, pH=6.5) containing varying concentrations of BA (1-10 mM), MA (10-100 mM) or PC (1-4 mM). The vials were then maintained on the nutating mixer at 60 rpm for 4 h, followed by separating the supernatants by centrifugation at 5000×g for 5 min and analyzing using the UV-vis spectrophotometer at λₘₐₓ=280 nm.

Effect of SIF on VAN removal: To investigate the effect of SIF on VAN removal, VAN solutions with varying concentrations (1-20 mg mL⁻¹) were prepared by diluting the VAN stock solutions with either FaSSIF or FeSSIF. The final volume of VAN solutions was 1 mL after the pH adjustment to 6.5 using a NaOH solution (0.5 M). AHCNC-CHA (20 mg) was then added to the VAN solutions, agitated at 60 rpm using the nutating mixer for 4 h. The supernatants were then separated by centrifugation at 5000×g for 5 min and analyzed by the UV-vis spectrophotometer at λₘₐₓ=280 nm.

High-performance liquid chromatography (HPLC): An HPLC instrument (Shimadzu LC-20AD UFLC XR, Japan), equipped with a UV detector was used to measure VAN concentrations below 100 mg L⁻¹. The mobile phase consisted of 0.1% v/v formic acid in Optima™ liquid chromatography-mass spectroscopy (LC-MS) grade water and formic acid in acetonitrile (0.1% v/v). Chromatographic separation was conducted using an Acuity UPLC BEH C18 analytical column (length: 150 mm, inner diameter: 2.1 mm, particle size: 1.4 μm) (Acuity, USA). The injection volume and the flow rate were 1 μL and 0.25 mL min-1, respectively. The column temperature was maintained at 55° C. The peak areas detected at 205 nm were defined as analytical signs.

Uncaptured VAN antibiotic activity via a broth microdilution assay assay: Broth microdilution assays were used to quantify the effects of AHCNC-CHA on the antibiotic activity of VAN against VSEfm. VAN removal was conducted in Milli-Q water at pH=6.5 using varying AHCNC-CHA doses and incubation time. After centrifugation at 16,300×g for 5 min, the supernatant of each sample was separated and transferred to 0.22 μm cellulose acetate tube filters. All assays were performed according to the guidelines of the Clinical Laboratory Standards Institute using a patient-derived VSEfm (minimum inhibitory concentration MIC=0.5 μg mL⁻¹). Twofold serial VAN dilutions were prepared using the initial VAN concentrations (i.e., the VAN concentrations before AHCNC-CHA-mediated capture). The bacterial cell density was quantified after 24 h of incubation at 35° C. using a plate reader (BioTek Synergy H1 Plate reader, Agilent Technology, USA). The optical density was recorded at a wavelength of 600 nm, referred to as OD₆₀₀. The data were fitted to a Hill function as previously described, with decreases in initial VAN concentrations leading to a right-shift in the bacterial growth curve.

Assessing the antibiotic activity of uncaptured VAN in vivo: The Pennsylvania State University Institutional Animal Care and Use Committee (PSU-IACUC, 47581) approved all animal procedures. Two previously described VREfm and VSEfm isolates were used in all experiments. VREfm clone BL00239-1 (MIC>64.0 μg mL⁻¹), expressing the vanA VAN resistance gene, was isolated from a patient bloodstream at the University of Michigan Hospital. VSEfm clone, BL00239-1S (Computed MIC=1.0 μg mL⁻¹, 95% Confidence interval=0.8-1.3 μg mL⁻¹) was derived through the in vivo mouse passage of BL000239-1 as previously described, with the loss of vanA gene expression confirmed by polymerase chain reaction (PCR). Adult, 8-week-old female outbred Swiss Webster mice were used for this study. Given the absence of data suggesting sex-differences in VAN biliary excretion, only female mice were used to minimize animal use in accordance with PSU-IACUC guidelines. Mouse sample sizes (n=5) were determined using Biomath (version 03/03) at a power of 0.8 and a 0.05 probability of type 1 error based on prior work with similar compounds. Mice were allowed to acclimatize to the on-site facility for at least 2 weeks under Biosafety Level (BSL)-2 housing conditions at 5 animals per microisolator cage prior to E. faecium inoculation and moved to individual cages thereafter. Sterile gowns, shoe covers, masks, eye protection, surgical bouffant, and double gloves were required for room entry. To prevent cross contamination when handling mice, outer gloves were changed, and all surfaces were thoroughly cleaned with 70% ethanol between cages. Mice were given irradiated feed (i.e., PicoLab Rodent Diet 20) and sterile water ad libitum.

To disrupt the intestinal microbiome and facilitate intestinal E. faecium colonization, mice were treated for 7 days with ampicillin (ad libitum; 0.5 g L⁻¹ in drinking water) prior to bacterial inoculation. VSEfm and VREfm inoculations were grown overnight in BHI broth at 35° C. Mice were subsequently inoculated via oral gavage with sterile saline (100 μL), containing approximately 10⁸ colony forming units (CFUs) at an approximately 1:20 ratio of VREfm (BL00239-1) to VSEfm (BL00239-1S). Serial dilution on Enterococcosel agar plates was used to confirm inoculation density (1.3×10⁸ CFUs) and composition (6% VREfm). Beginning the day of E. faecium inoculation, mice were treated twice daily via oral gavage with either sterile saline (100 μL), sterile saline (100 μL)+AHCHC-CHA (4 mg), or sterile saline (100 μL)+AHCNC-CHA (10 mg) for 8 days. Beginning 1 day post inoculation, mice received veterinary grade VAN (30 mg kg⁻¹) diluted in saline (0.9% w/v) via subcutaneous injection once daily for 5 days. Body mass was recorded daily for each mouse to monitor the safety and tolerability of the AHCNC-CHA compound, and fecal samples were collected at preselected time-points by placing mice in fresh unsealed plastic containers until defecation. Fecal pellets were subsequently transferred to pre-weighed 1.5 mL tubes by autoclaved sterile toothpick, and fecal samples were dissolved in PBS (25 μL PBS mg⁻¹ feces) prior to freezing at −80° C. in glycerol (25%) until analysis by plating.

Total fecal E. faecium concentrations were quantified by serially diluting the previously frozen samples tenfold in PBS before plating on Enterococcosel agar and incubation for 40-48 h under aerobic conditions at 35° C. E. faecium were identified by the ability to grow on Enterococcosel agar and colony morphology with each colony assumed to represent a single viable bacterium (CFU; colony forming unit) in the original sample. CFU counts were back calculated against the corresponding dilution factor to determine the total E. faecium concentrations in the initial fecal samples. Total CFU counts were normalized to CFUs per 10 mg of feces, and the limit of detection for the total E. faecium calculation was 5 CFUs per 10 mg of feces.

To enumerate fecal VREfm density, the calculated total E. faecium densities were used to plate 200 CFUs in triplicate on selective agar plates, supplemented with or without VAN (16 μg mL⁻¹) for a total of 6 plates for each sample. Plates were then incubated for 40-48 h at 35° C., and the difference in CFU counts between plates with or without VAN were used to quantify the proportion of VREfm and VSEfm in each sample. Cumulative total E. faecium, VREfm, and VSEfm shedding was calculated from the area under the fecal density time series (Area Under the Curve: AUC) for the duration of the study (14 days).

Statistical Analyses: All in vitro data were collected from at least three independent experiments and reported as mean±standard deviation (SD). All in vivo data represent the mean±standard error of the mean (SEM). Significant differences between mean values were determined by the non-parametric Kruskal-Wallis test, followed by Dunn's post-hoc test for pairwise multiple comparisons. Statistical analyses were performed using GraphPad Prism software (version 10.0.3), and mean differences were considered significant at p-values<0.05. The symbols *, **, ***, and ***** represent p-values of p<0.05, p £ 0.01, p £ 0.001, and p £ 0.0001, respectively. The p-values of p ³ 0.05 are considered non-significant.

### Results

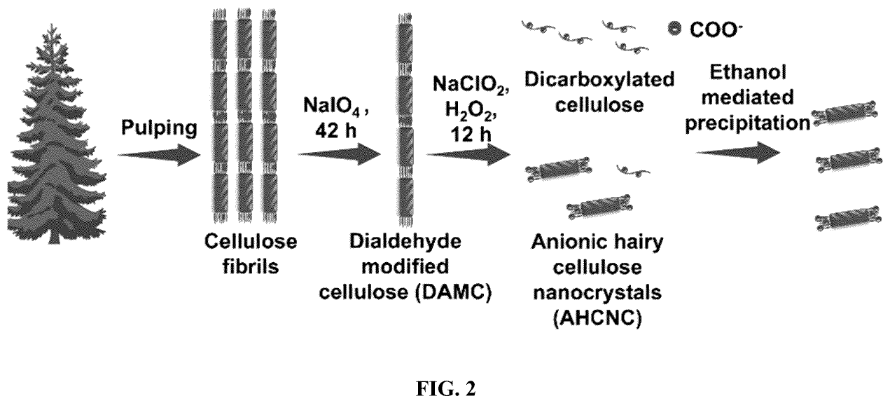

Design of AHCNC-CHA anti-antibiotics: Our process is based on the oral administration of AHCNC-CHA sorbent for GI-exposed VAN removal. To implement this approach, we first engineered highly negatively charged AHCNC, providing active sites for VAN removal, and hybridized it with CHA, yielding the AHCNC-CHA sorbents. FIG. 2 shows the synthesis of AHCNC from softwood cellulose pulp fibrils through sequential oxidation reactions. The first 42 h periodate reaction yielded intact DAMC fibrils, which were further converted to a mixture of dicarboxylate-bearing AHCNC and DCC via a 12 h chlorite oxidation reaction. AHCNC were then isolated by nonsolvent-mediated precipitation.

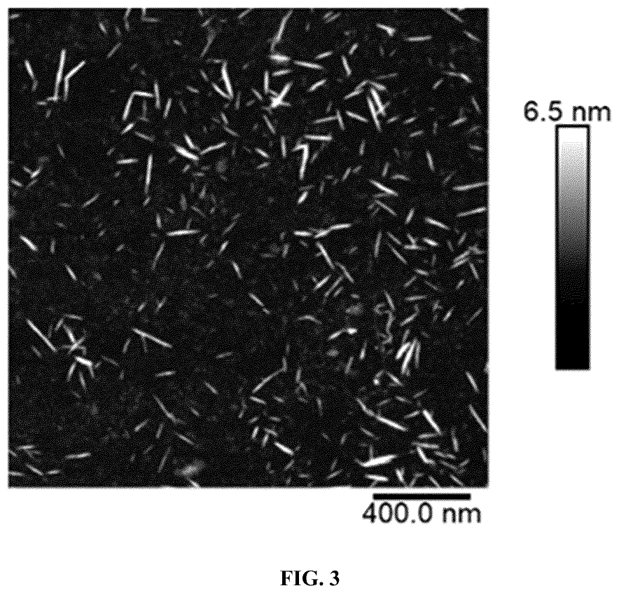

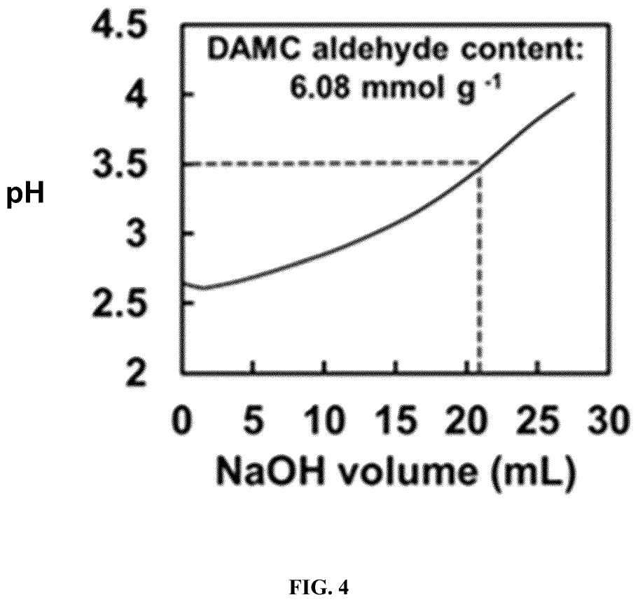

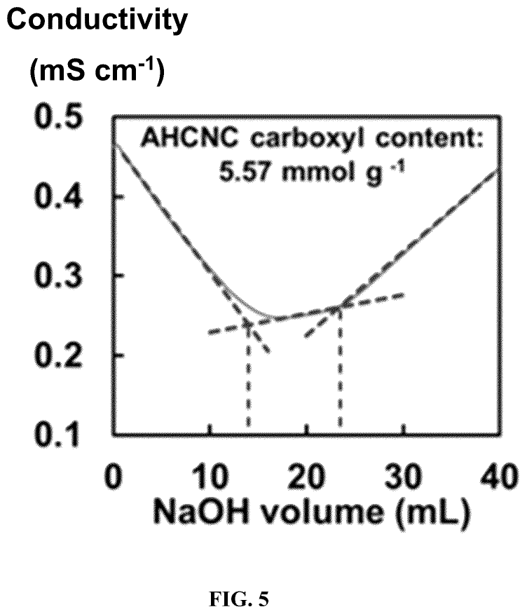

FIG. 3 presents the needle-like crystalline body of AHCNC, obtained by AFM imaging. The length and width of AHCNC were 96±26 nm and 7±2 nm, respectively, i.e., within the same order of magnitude of conventional (strong-acid hydrolyzed, non-hairy) CNCs. It has been indirectly shown that AHCNC comprise a crystalline body with polyanionic disordered cellulose chains protruding from both ends. The aldehyde content of DAMC, an intermediate product for the AHCNC synthesis, was determined by measuring the NaOH volume required to neutralize HCl released as the aldehyde groups reacted with hydroxylamine-hydrochloride. The aldehyde content was 6.0±0.3 mmol g⁻¹ (an example shown in FIG. 4), which were then converted to carboxylate groups to disintegrate DAMC and form AHCNC. The carboxylate content of AHCNC was 6.0±0.4 mmol g⁻¹, which was assessed by the conductometric titration (an example shown in FIG. 5).

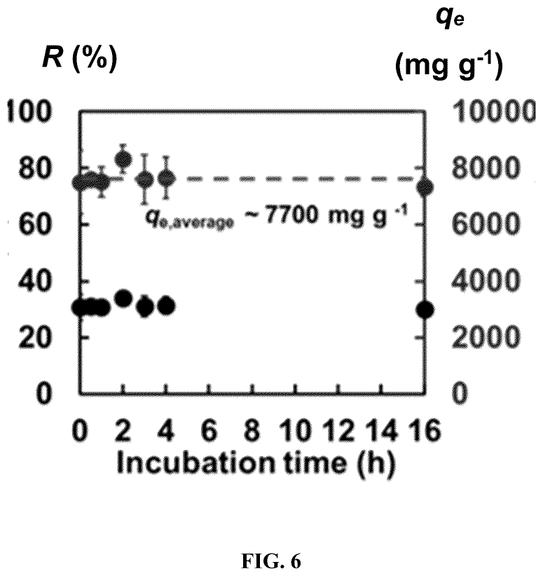

AHCNC, bearing a high content of polyanionic groups, were used as the active anti-antibiotic component. Assuming the adsorption of VAN to AHCNC is electrostatically driven, 5.57 mmol of carboxylate groups per 1 g of AHCNC adsorbs an equal molar amount of amine groups on VAN (theoretical amine group content of VAN is 1.34 mmol g⁻¹); thus, 0.25 mg mL⁻¹ of AHCNC stoichiometrically binds 1.11 mg mL⁻¹ of VAN. To investigate the AHCNC-mediated VAN removal kinetics, FIG. 6 shows the VAN removal percentage (R) and capacity (qₑ) of AHCNC as a function of incubation time. AHCNC (0.25 mg mL⁻¹) were incubated with a supra-stochiometric VAN concentration (6 mg mL⁻¹, 1 mL) to identify the effect of contact time on VAN removal. In this condition, R~30% and qₑ~7700±1280 mg of VAN per 1 g of AHCNC was achieved in less than 10 s. The qₑ obtained here was ~2 times higher than the stoichiometric value, 4038±290 mg g⁻¹, based on the charge stoichiometry. The higher experimental qₑ may imply that the electrostatic interactions of only one VAN amine group (out of two per molecule) with one carboxylate group of AHCNC may be sufficient for AHCNC-mediated VAN removal.

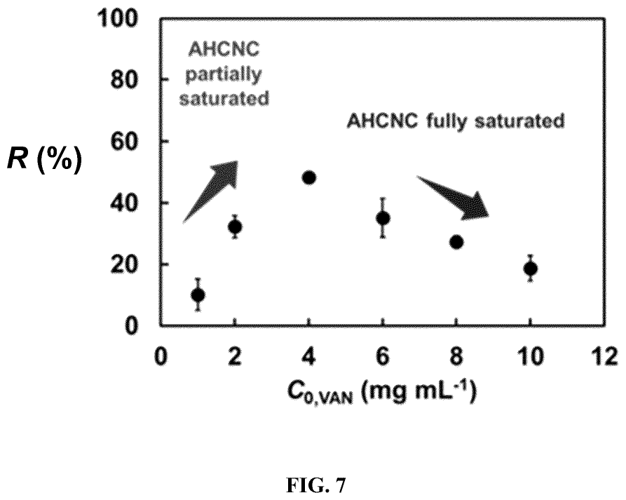

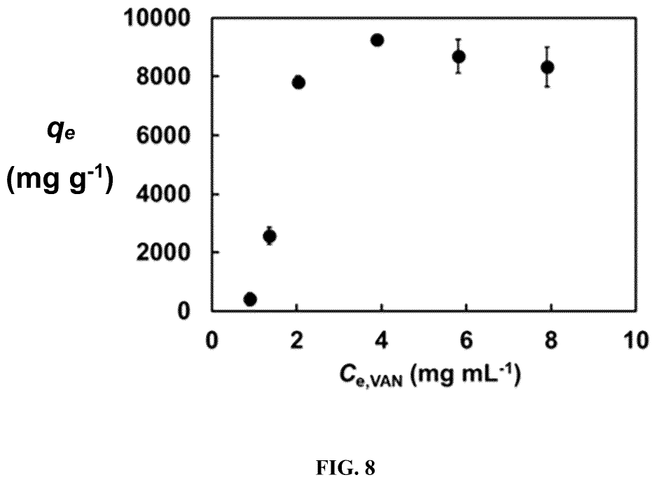

FIG. 7 presents the effect of initial VAN concentration on AHCNC R. Interestingly, R~10% at an initial VAN concentration of ~1 mg mL⁻¹, despite the high qₑ of AHCNC. Nanoscale charged sorbents, such as AHCNC, do not undergo colloidal aggregation when the adsorbate concentration is stoichiometrically lower than the sorbent charged groups, i.e., the sorbent charge is not neutralized. Accordingly, the AHCNC-VAN aggregates remained colloidally stable in the solution when AHCNC were only partially saturated with VAN. R reached a maximum value (~48%) when the initial VAN concentration was ~4 mg mL⁻¹, but decreased to 19% at 10 mg mL⁻¹ of VAN, because the active binding sites (i.e., dicarboxylate groups) of AHCNC were saturated with VAN. FIG. 8 shows VAN qₑ of AHCNC versus the equilibrium VAN concentration (Cₑ). The qₑ increased as equilibrium VAN concentration increased to 4 mg mL⁻¹, reaching a plateau of ~8316±752 mg g⁻¹.

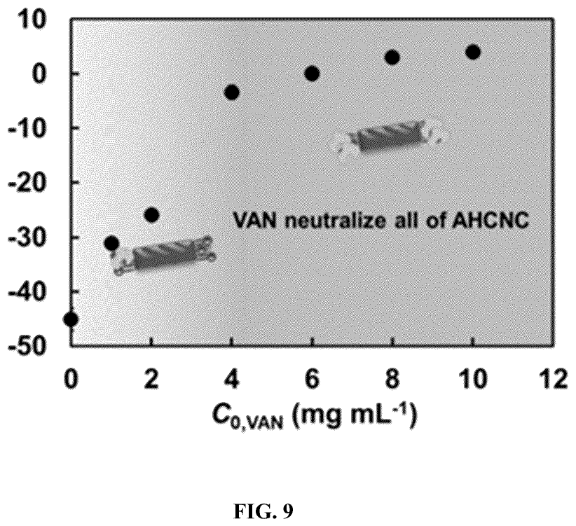

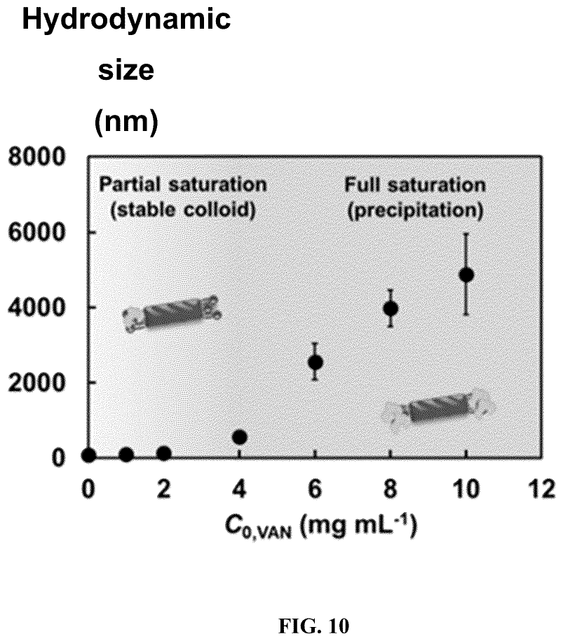

FIGS. 9-10 present the ζ-potential and hydrodynamic equivalent size of AHCNC (1 mg mL⁻¹) at varying initial VAN concentrations (pH=6.5), respectively. The absolute value of AHCNC ζ-potential decreased and reached ~0 by increasing the initial VAN concentration from 0 to 4 mg mL⁻¹, confirming electrostatic interactions between AHCNC and VAN causing AHCNC charge neutralization. The hydrodynamic size of AHCNC did not significantly change (~100 nm) when the initial VAN concentrations increased from 0 to 2 mg mL⁻¹; however, it reached >2000 nm as a result of colloidal aggregation, and precipitates were observed at initial VAN concentrations>4 mg mL⁻¹.

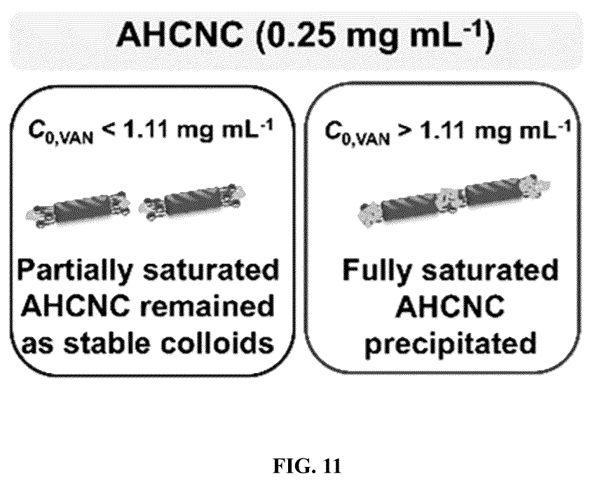

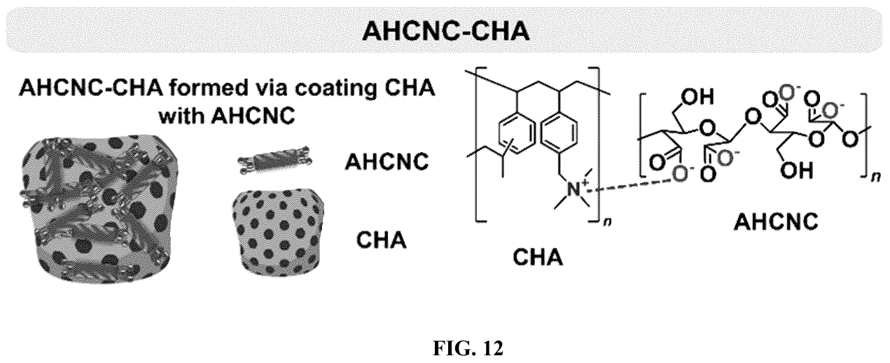

FIG. 11 schematically shows the colloidal behavior of AHCNC sorbent when they are partially or fully saturated with VAN. Given that 1.11 mg mL⁻¹ of VAN neutralizes 0.25 mg mL⁻¹ of AHCNC, AHCNC were only partially saturated with VAN at initial VAN concentrations below 1.11 mg mL⁻¹. The partially saturated AHCNC remained colloidally stable in the solution without undergoing significant phase separation, and became fully saturated with VAN at the initial VAN concentrations>1.11 mg mL⁻¹, inducing phase separation and precipitation. Accordingly, VAN is not completely removed by AHCNC at low VAN concentrations (C₀<1.11 mg mL⁻¹). Importantly, the physiological VAN concentration in the GI tract is much lower than this value, limiting the in vivo translatability of AHCNC without using a substrate.

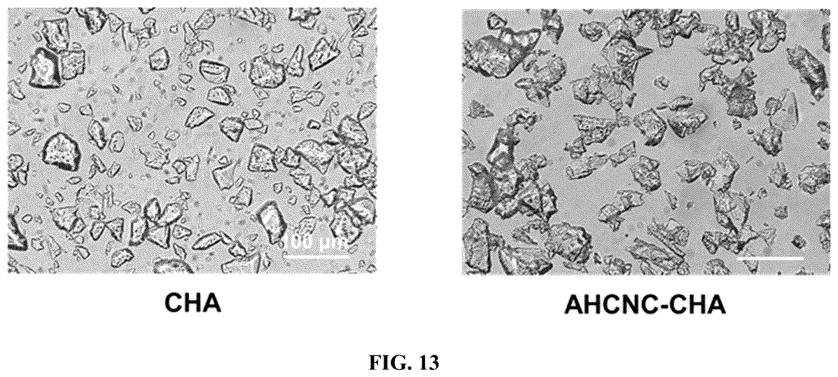

To overcome the colloidal stability limitation of AHCNC at low adsorbate-to-adsorbent ratios, it was coated on CHA to enable VAN separation at the low VAN concentrations. FIG. 12 presents the schematic of coating AHCNC on CHA via electrostatic interactions between AHCNC carboxylate groups and CHA quaternary ammonium groups, yielding a new bio-based sorbent, referred to as “AHCNC-CHA”. The binding energy between carboxylate groups and quaternary ammonium groups is ~71.2 kJ mol⁻¹, resulting in AHCNC-CHA stability in the physiological conditions of GI tract. FIG. 13 presents the optical microscopy images of CHA and AHCNC-CHA microscale particles. FIG. 1 summarize the concept of using AHCNC-CHA as an “anti-antibiotic” adjuvant therapy versus not using it. If VAN remains in the intestine (FIG. 1), it may drive the VSEfm to resistant mutants, i.e., VREfm, which enter the bloodstream by moving across the intestinal lining and passing through the liver, causing infection. However, if anti-antibiotic is orally taken by IV VAN-administrated patients, it provides an orthogonal remedy to remove excess VAN from the GI tract and to prevent the resistance pressure on the VSEfm, eliminating the emergence of VREfm.

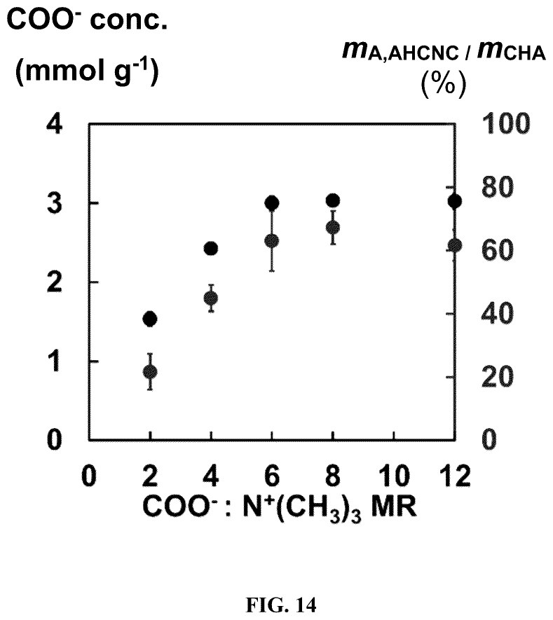

Optimization of AHCNC-CHA anti-antibiotic composition: To maximize VAN removal capacity, AHCNC-CHA was fabricated at varying molar ratios of dicarboxylate groups of AHCNC to quaternary ammonium groups of CHA, ranging from 2:1 to 12:1, i.e., AHCNC concentrations varying from 3.3 mg mL⁻¹ to 20 mg mL⁻¹ with a constant CHA concentration of 20 mg mL⁻¹. FIG. 14 presents the net carboxylate content of AHCNC-CHA and mass of adsorbed AHCNC, defined as the mass of adsorbed AHCNC to the mass of CHA, at varying COO⁻:quaternary ammonium (N⁺(CH₃)₃) molar ratios. The carboxylate content and mass of adsorbed AHCNC increased by increasing the molar ratio from 2 to 6 and reached a plateau (carboxylate content~3 mmol g⁻¹ and mass of adsorbed AHCNC=80%) at higher stoichiometric ratios, as a result of CHA saturation with AHCNC.

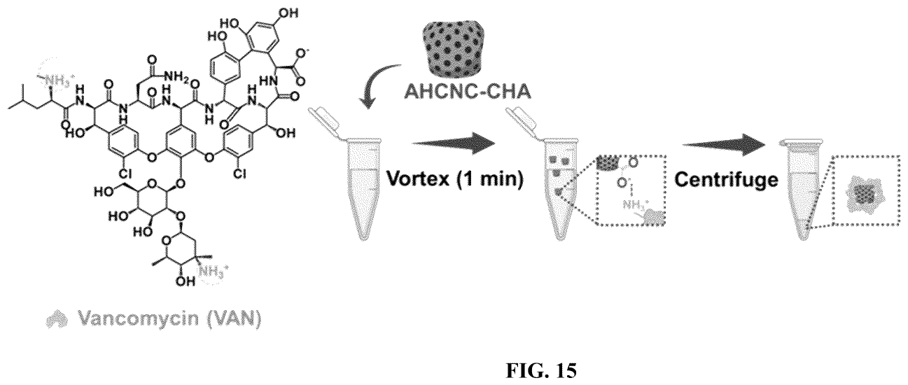

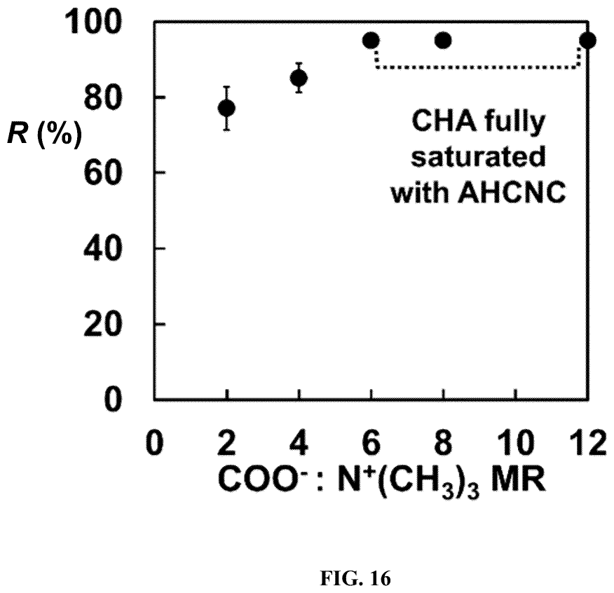

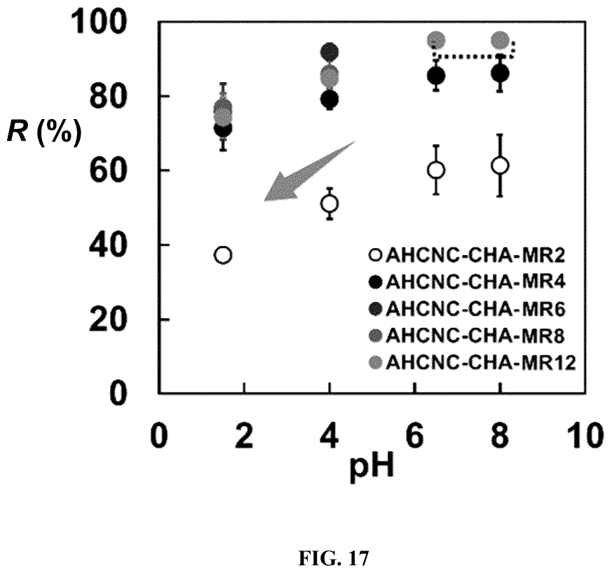

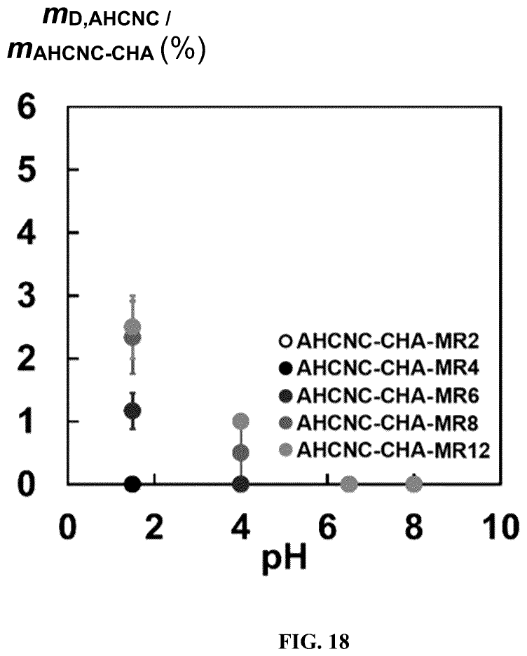

To examine the VAN removal performance of AHCNC-CHA compositions, a desired amount of each compound was incubated with VAN in varying media, as schematically shown in FIG. 15. FIG. 16 shows the VAN (1 mg mL⁻¹) R of AHCNC-CHA prepared at varying COO⁻:(N⁺(CH₃)₃ stoichiometric ratios via changing varying AHCNC concentrations. R~95% when the AHCNC-CHA stoichiometric ratio was above 6, resulting in the highest carboxylate group content, which electrostatically interacted with the amine groups of VAN. To assess the stability of AHCNC-CHA, FIG. 17 presents the R of AHCNC-CHA, prepared at varying COO⁻:(N⁺(CH₃)₃) stoichiometric ratios, after incubating AHCNC-CHA at varying pH ranging from 2 to 12 for 6 h (beyond the food residence time in the GI tract). For convenience, we referred to the AHCNC-CHA formulated with varying stoichiometric ratios (SR) as AHCNC-CHA-SRn (n=2, 4, 6, 8, 12). The R for all AHCNC-CHA decreased ~20% by decreasing pre-removal incubation pH from 6.5 to 1.5. This may be explained by two possibilities: (i) the “hairs” on AHCNC are partially hydrolyzed at extremely low pH; (ii) AHCNC is partially desorbed from the CHA as a result of carboxylate group partial protonation at extremely low pH. FIG. 18 shows the ratio of desorbed AHCNC mass from AHCNC-CHA with respect to initial AHCNC-CHA mass after incubating the AHCNC-CHA at varying pH for 6 h. As the desorbed AHCNC was <5% at this pH range, the first possibility was more likely to be the main reason for the reduction in VAN removal.

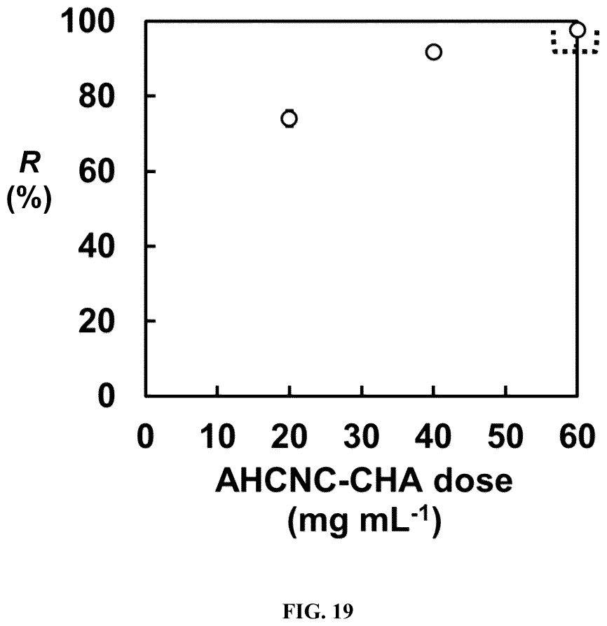

Accordingly, AHCNC-CHA-SR6 bears the highest charge content, GR, R, and stability in acid media at the lowest possible AHCNC content. Therefore, it was selected as the optimal anti-antibiotic compound. For simplicity, we abbreviated AHCNC-CHA-SR6 as AHCNC-CHA using which the rest of this study was conducted. The VAN R of AHCNC-CHA after 6 h pre-removal incubation of AHCNC-CHA at pH=1.5 was also studied and presented in FIG. 19. Although R decreased to 74% by decreasing pre-removal pH to 1.5 (FIG. 17), >95% of VAN was removed by increasing AHCNC-CHA concentration (dose).

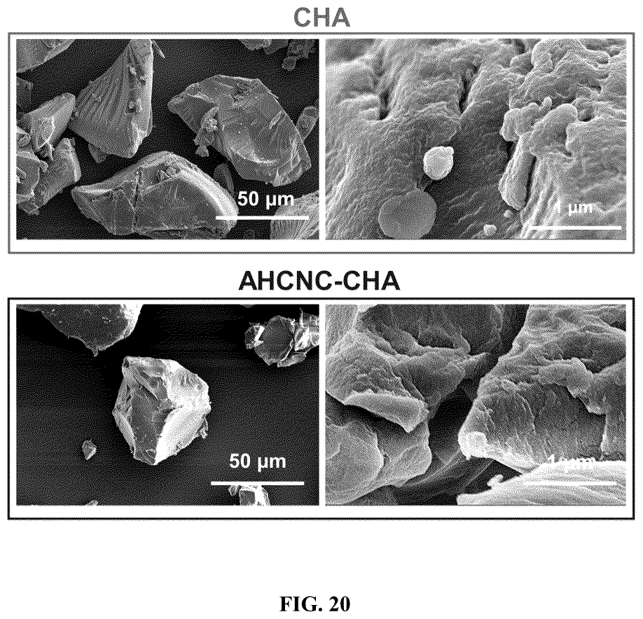

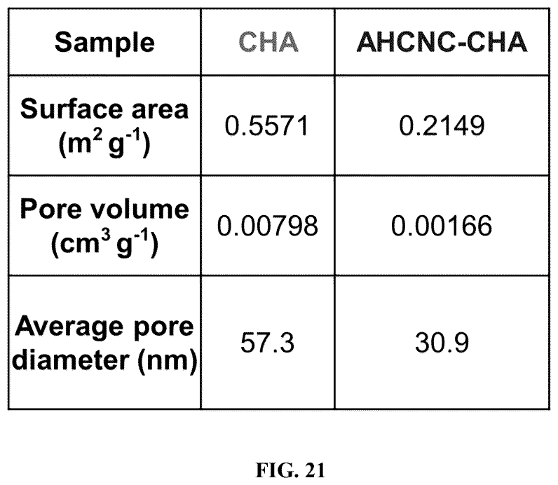

AHCNC-CHA was thoroughly characterized based on surface morphology, pore properties, and functional groups, as observed in FIGS. 20-22, via ATR-FTIR, SEM, and porosimetry, respectively. FIG. 20 shows the SEM images of CHA and AHCNC-CHA. The small width of nanocellulose (7±2 nm, obtained from AFM images) renders the AHCNC visualization by SEM imaging non-trivial. CHA and AHCNC-CHA had similar particle size, topography, and surface features (wrinkles and rugged regions) within a micromere length scale. FIG. 21 presents the surface area, average pore diameter, and pore volume of CHA and AHCNC-CHA. The reduction in average diameter (d) and volume (v) of AHCNC-CHA (d=30.9 nm, v=0.00166 cm³ g⁻¹) pores compared with CHA (d=57.3 nm, v=0.00798 cm³ g⁻¹) may be a result of AHCNC adsorption to the surface and within the pores of CHA.

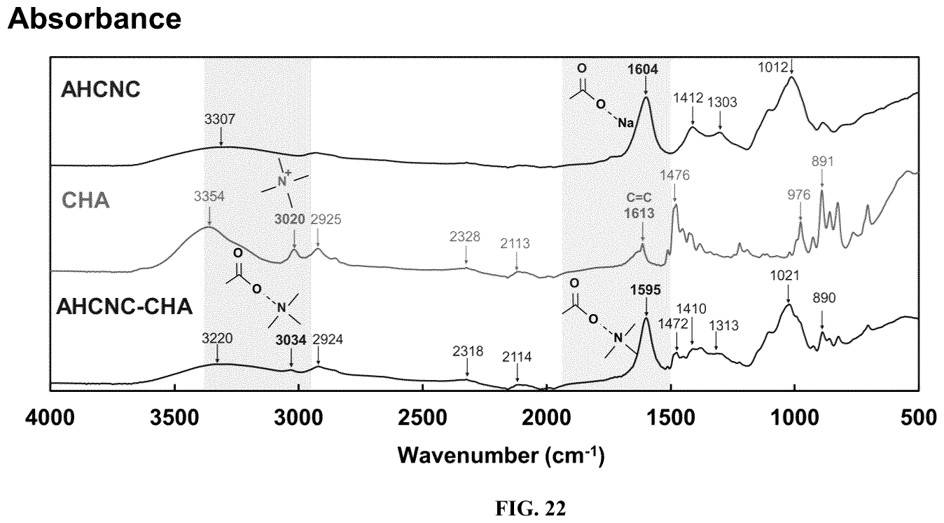

To confirm the AHCNC coating on CHA, FIG. 22 shows the ATR-FTIR spectra of AHCNC, CHA, and AHCNC-CHA. The peak at 3019 cm⁻¹ in CHA spectrum was attributed to the C-H stretching of quaternary ammonium groups. Shifting C-H stretching peak to 3030 cm⁻¹ in the AHCNC-CHA spectrum may be a result of the electrostatic binding of CHA quaternary ammonium groups to AHCNC carboxylate groups. The peak at 1602 cm⁻¹ in AHCHC spectrum was assigned to the stretching vibrations of carbonyl groups, pertaining to carboxylate (i.e., carboxyl group in the sodium form). In the AHCHC-CHA spectrum, this peak shifted to 1592 cm⁻¹, likely because of the interactions of quaternary ammonium and carboxylate groups.

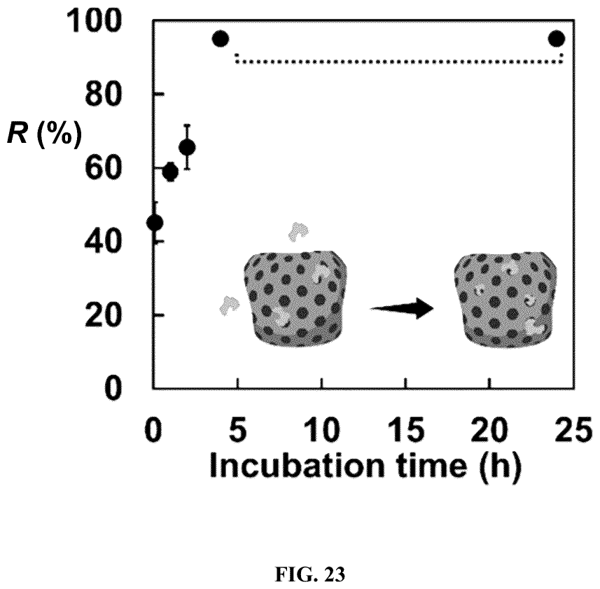

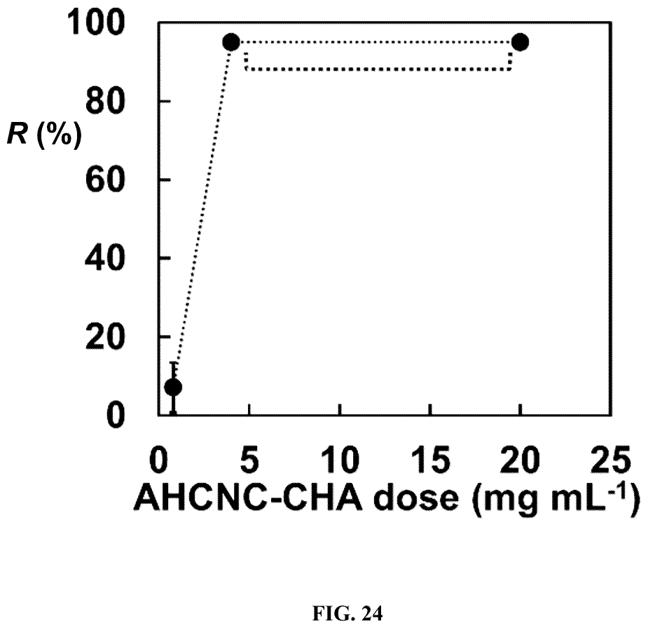

VAN removal at varying incubation times was studied to investigate the kinetics of AHCNC-CHA-mediated antibiotic capture. FIG. 23 shows the VAN R of AHCNC-CHA versus incubation time. The R reached ~40% almost immediately and increased to ~95% after 4 h of incubation. The time-dependent adsorption process may be associated with VAN molecular diffusion in the AHCNC-CHA pores, resulting from the porous structure of AHCNC-CHA. FIG. 24 presents the R at varying AHCNC-CHA doses, showing an increase from R~7% to ~95% by increasing AHCNC-CHA dose from 0.8 to 20 mg mL⁻¹, which confirms that the VAN removal process is dose dependent.

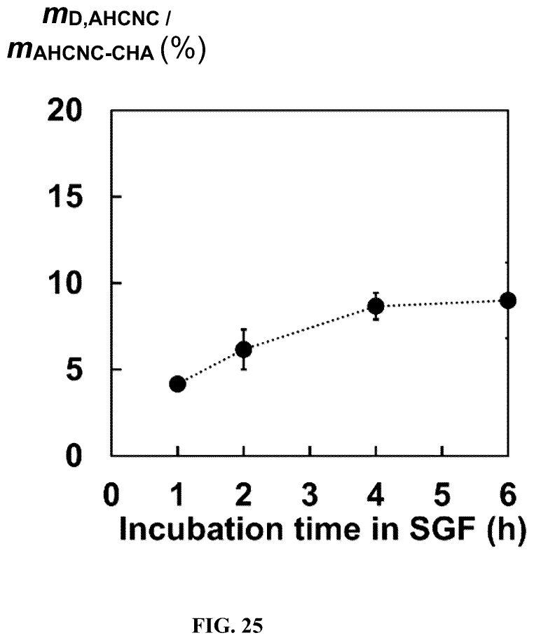

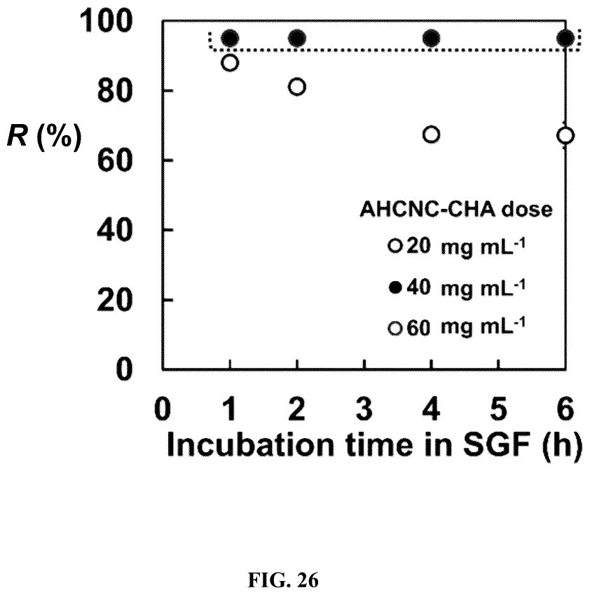

As the anti-antibiotic is an oral adjuvant, it must be able to pass through the stomach (pH~1.5, 1 h<residence time<4 h) before reaching the intestines. Therefore, the stability of AHCNC-CHA was assessed in the simulated gastric condition. FIG. 25 shows the desorption ratio of AHCNC from AHCNC-CHA after incubating in the SGF for varying periods. By increasing the incubation time from 1 h to 6 h, the desorption ratio increased from 4±1% to 9±2%, respectively. The AHCNC desorption ratio was less than 10% at the extreme acidic condition of SGF after 6 h. FIG. 26 presents the VAN R of SGF-incubated AHCNC-CHA. Although the R dropped by ~12% after 1 h and ~33% after 4 h of incubation in SGF because of the acid-mediated partial hydrolysis of poly anionic hairs, by increasing the dose of AHCNC-CHA to 60 mg mL⁻¹, the R reached ~95%.

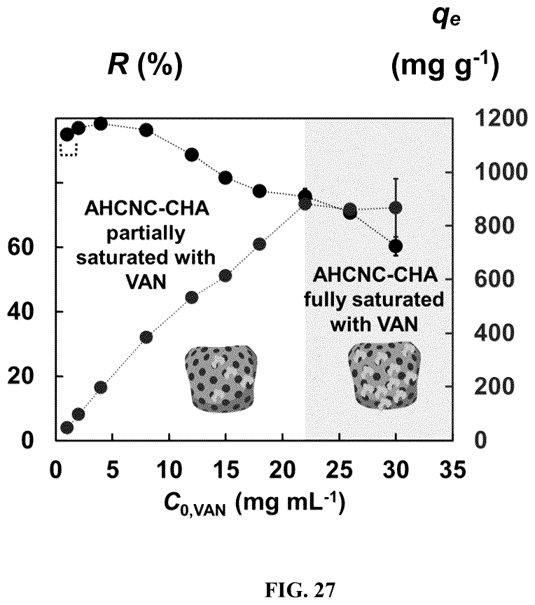

AHCNC-CHA-mediated VAN removal: To uncover the interactions between AHCNC-CHA and VAN, the effects of initial VAN concentrations, pH, and ionic strength on the R and qₑ of AHCNC-CHA were studied. FIG. 27 shows R and qₑ at varying initial VAN concentrations (1-30 mg mL⁻¹). At initial VAN concentrations ranging from 1 to 8 mg mL⁻¹, the R was ~95%. The molar stoichiometric ratio of AHCNC-CHA carboxylate groups to VAN amine groups was 10:0.125 at initial VAN concentration=1 mg mL⁻¹, which reached 10:1 at initial VAN concentration=8 mg mL⁻¹, enabling ~100% removal of VAN. When the initial VAN concentration increased beyond 8 mg mL-1, R decreased as a result of VAN-induced AHCNC-CHA carboxylate group saturation (neutralization). The qₑ increased as the initial VAN concentration increased from 1 mg mL⁻¹ to 22 mg mL⁻¹, reaching a plateau at ~850 mg g⁻¹ at higher initial VAN concentrations. The maximum qₑ in this condition was ~38% lower than the theoretical value based on the charge stoichiometry, i.e., 3 mmol of AHCNC-CHA carboxylate groups adsorbs 3 mmol (out of 5) of VAN amine groups, corresponding to 2239 mg g⁻¹ of VAN. The lower qₑ compared with the theoretical value may be because not all carboxylate binding sites on AHCNC-CHA electrostatically interacted with VAN as a result of steric hindrance.

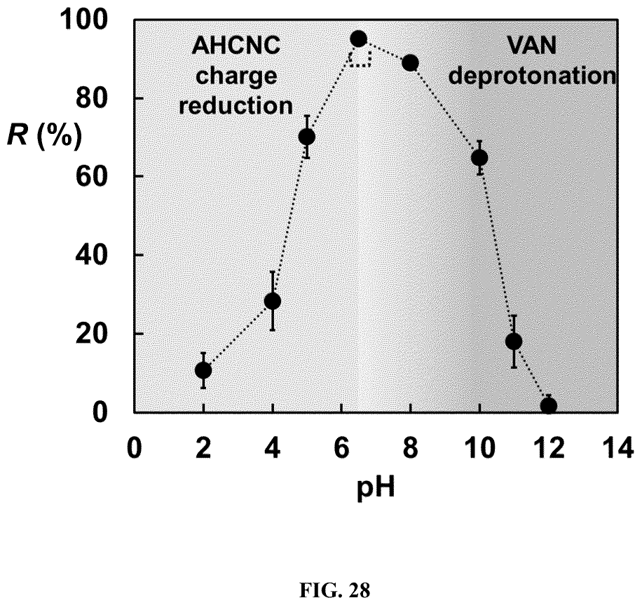

FIG. 28 presents AHCNC-CHA-mediated VAN R at varying pH. By adjusting the pH to more acidic or alkaline conditions, R decreased. At pH<pK_{a,2}=7.5, AHCNC-CHA carboxylate groups become protonated, decreasing the charge content and reducing the number density of binding sites. At pH>7.5, the amine groups of VAN become deprotonated, reducing the positive charges, which in turn decreases the number of electrostatic binding sites. Accordingly, R decreased to ~65% at pH~10, at which condition VAN and AHCNC-CHA bear net negative charges. The non-zero removal was possibly associated with the local positive charges on VAN, interacting with negatively charged AHCNC-CHA at pH~10. These results suggested that the adsorption of VAN to AHCNC-CHA anti-antibiotic is governed by electrostatic attraction.

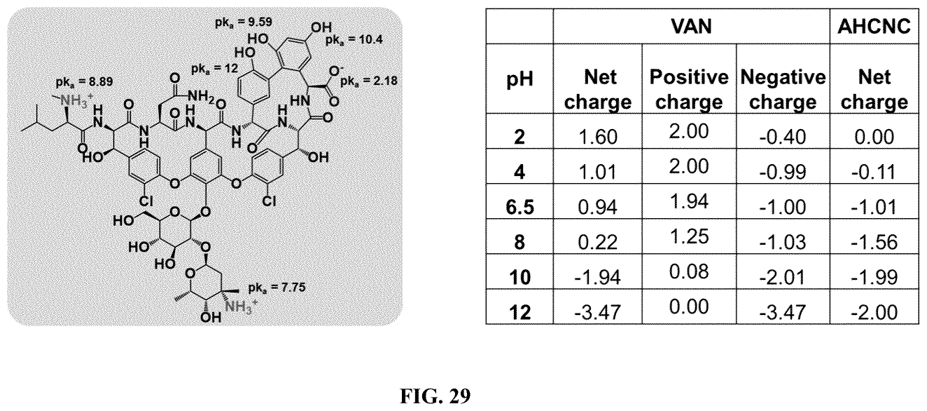

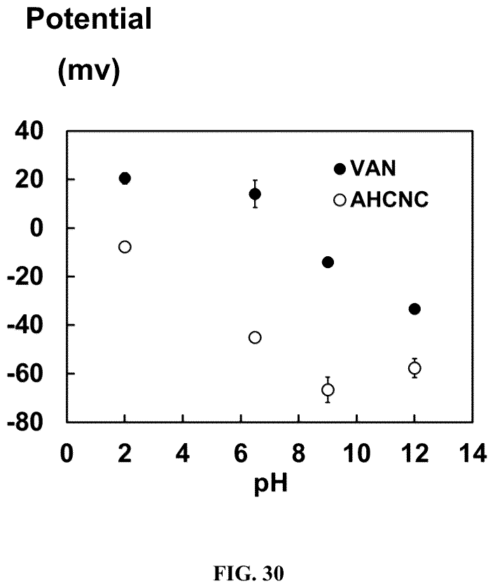

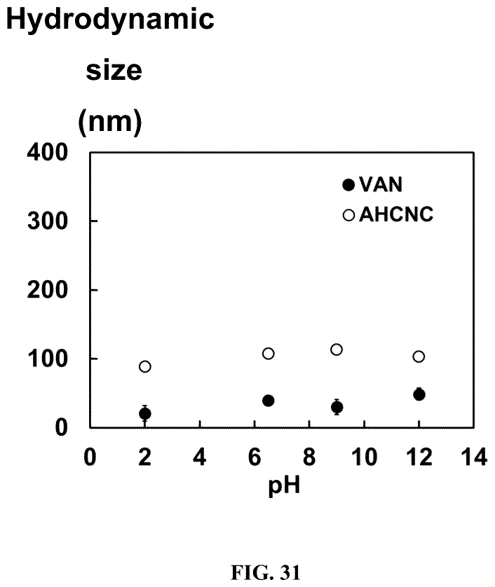

FIG. 29 shows the chemical structure of VAN, including the pKₐ values of its major functional groups, as well as the net charge of VAN and AHCNC at varying pH based on their ionization states. The net charge of VAN is positive at pH ranging from 2-8, whereas its carboxylate groups are deprotonated, rendering the molecule anionic at pH>8. FIGS. 30-31 present the ζ-potential and hydrodynamic equivalent size of VAN and AHCNC at varying pH, respectively. The ζ-potential of VAN changed from 20±2 mV at pH=2 to −33±1 mV at pH=12, justifying the reduction in R at alkaline pH (≥8) wherein the ζ-potential of AHCNC was also negative. The hydrodynamic size of AHCNC and VAN changed from 89±1 nm and 21±6 nm at pH=2, respectively, to 10⁴±2 nm and 49±3 nm at pH=12.

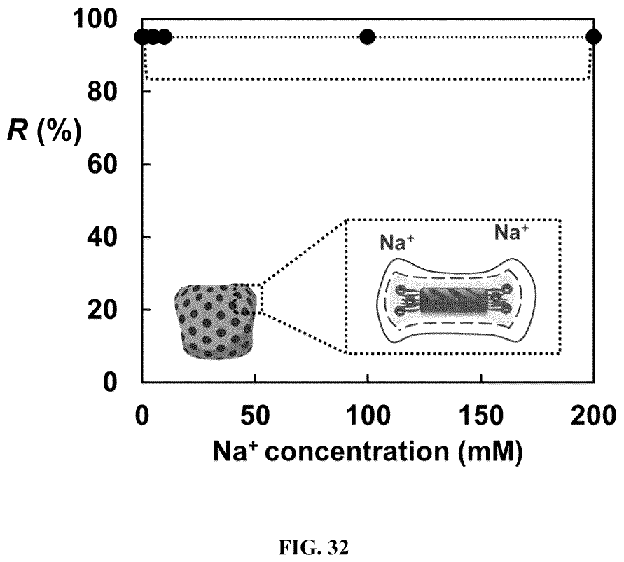

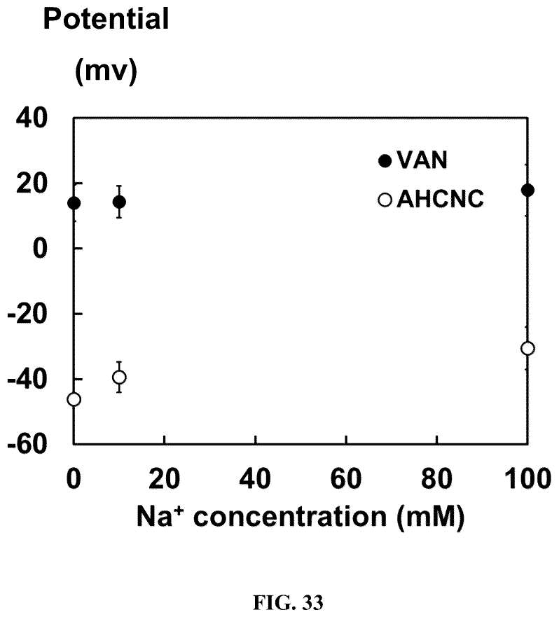

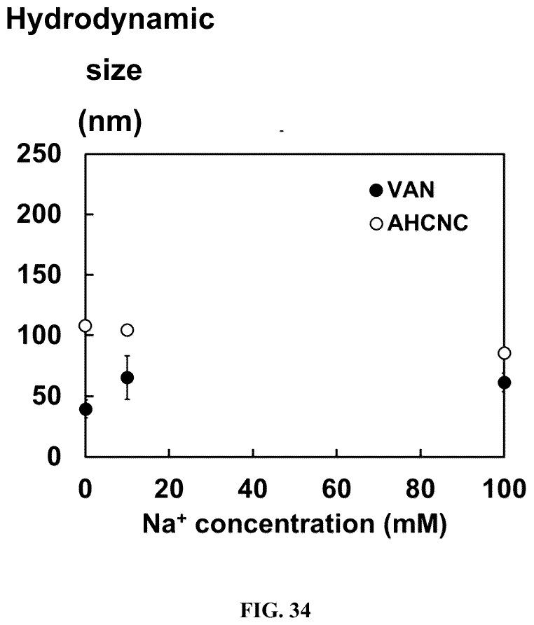

FIG. 32 shows the R and qₑ at varying sodium ion (Nat) concentrations. R did not significantly change as the Na⁺ concentration increased from 0 to 100 mM. This was explained by investigating the ζ-potential and hydrodynamic size of VAN and AHCNC at varying Na⁺ concentrations, as presented in FIGS. 33-34, respectively. The addition of Na⁺ did not significantly change the ζ-potential or hydrodynamic size of VAN and decreased the AHCNC ζ-potential from −46±1 to −31±6 mV as a result of electrical double layer thickness reduction without compromising the AHCNC surface charge. Accordingly, the AHCNC-CHA anti-antibiotic remained charged and capable of binding VAN.

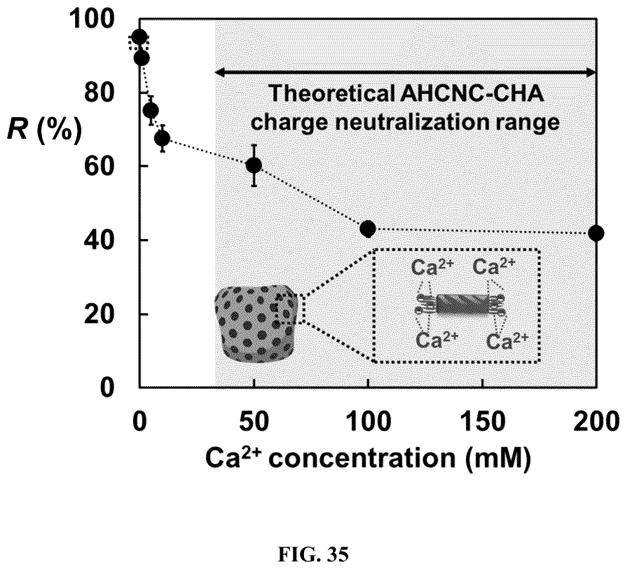

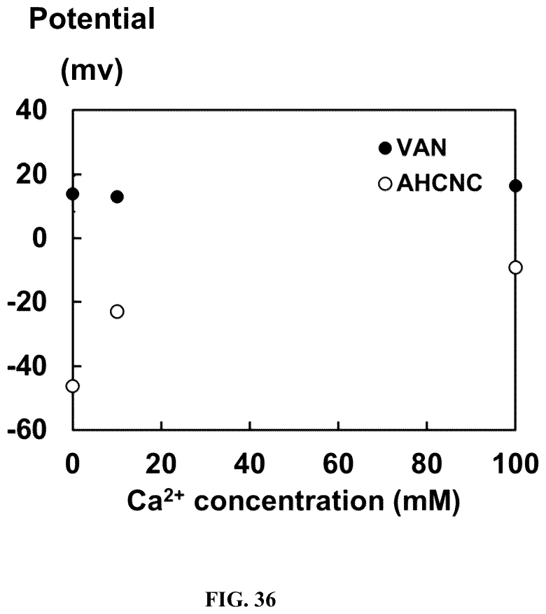

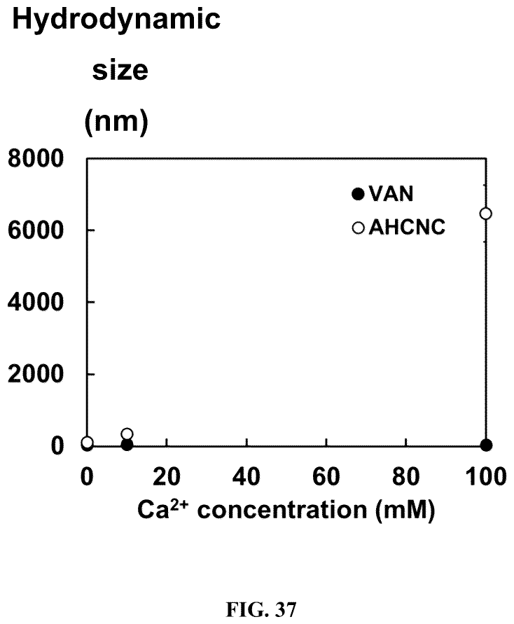

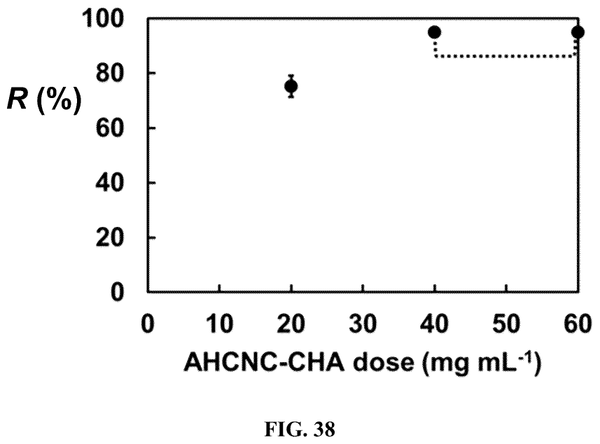

FIG. 35 shows R and qₑ at varying Ca²⁺ concentrations. Increasing the Ca²⁺ concentration from 0 mM to 200 mM results in ~60% reduction in R as a result of AHCNC charge neutralization by the divalent cation (Ca²⁺:COO⁻=1 mol: 2 mol). FIGS. 36-37 present the ζ-potential and hydrodynamic equivalent size of VAN and AHCNC at varying Ca²⁺ concentrations, respectively. The ζ-potential (~14±6 mV) and hydrodynamic size (~40±7 nm) of VAN remain unchanged by increasing Ca²⁺ concentration from 0 mM to 100 mM, which is because VAN and Ca²⁺ are similarly charged. AHCNC ζ-potential decreases from −46±1 mV to −9±1 mV, and the hydrodynamic equivalent size increases from 10⁸±1 nm to 6472±791 nm, by increasing Ca²⁺ concentration from 0 mM to 100 mM. These observations are attributed to Ca²⁺-mediated neutralization of AHCNC carboxylate groups and colloidal aggregation, respectively. Notably, ~42% of VAN is removed when the Ca²⁺ concentration is ~200 mM, at which condition the AHCNC should theoretically be fully neutralized (30 mM of Ca²⁺ stoichiometrically neutralizes 60 mM of carboxylate groups on 20 mg mL⁻¹ of AHCNC-CHA). The incomplete neutralization of AHCNC carboxylate groups may be attributed to the disordered dicarboxylate hairs closer to the crystalline region (inner hairs), which are likely less accessible or more structurally constrained compared to the outer hairs. To examine the effect of AHCNC-CHA dose on the antibiotic removal at the physiological concentration range of Ca²⁺ (~2-6 mM), FIG. 38 presents VAN R at 5 mM of Ca²⁺ and varying AHCNC-CHA doses. The initial reduction of R as a result of competitive Ca²⁺ adsorption is compensated by increasing the AHCNC-CHA dose to 40 mg mL⁻¹, yielding R~95%.

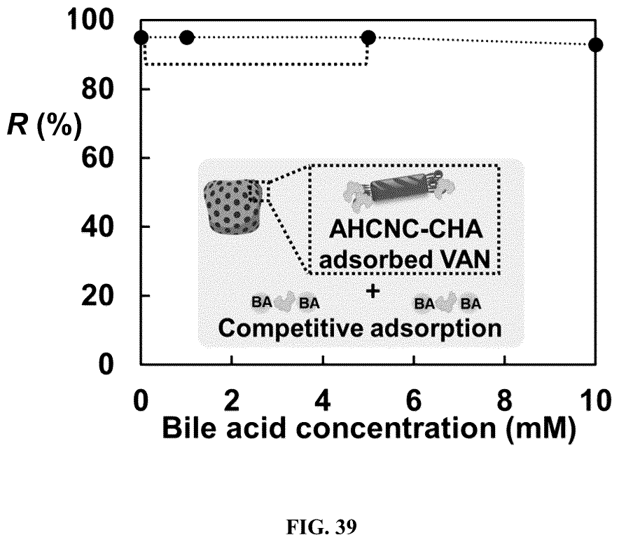

In vitro AHCNC-CHA anti-antibiotic-mediated VAN removal in the SIF: SIF contains BA, MA, and PC, which may interact with VAN or AHCNC-CHA anti-antibiotic. To emulate VAN removal from the intestines, we investigated the individual and combined effects of SIF components on AHCNC-CHA-mediated VAN removal in vitro. FIG. 39 presents R at varying BA concentrations, ranging from 3 mM to 10 mM, i.e., the physiological BA concentration range in FaSSIF to FeSSIF. The R decreased by ~8% by increasing BA concentration form 0 mM to 10 mM as a result of the competition between BA and AHCNC-CHA anti-antibiotic to electrostatically bind VAN. The negatively charged sulfonate groups of BA may bind VAN, forming colloidally stable BA-VAN aggregates, thereby reducing R. Since the sulfonate groups are more electronegative than carboxylate, they have a higher affinity and binding strength to amine groups. Based on the charge stoichiometry, 10 mM of BA (containing 10 mM sulfonate groups) can electrostatically bind all VAN (1 mg mL⁻¹=1.379 mM protonated amine groups), which was in agreement with the experimental R (~92%).

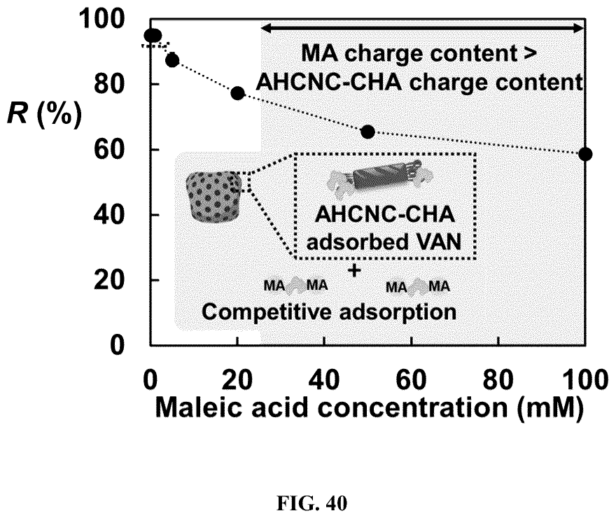

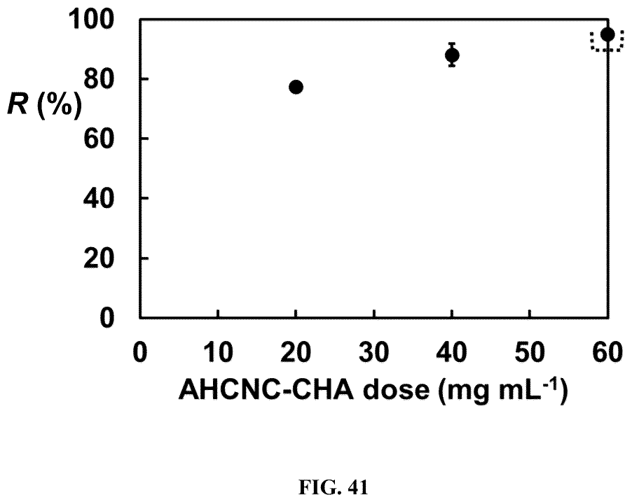

FIG. 40 shows R at varying MA concentrations ranging from 5 mM to 100 mM (physiological MA concentration in SIF~19 mM). The R decreased by ~23% at 20 mM MA because of the competition of carboxylate-bearing MA with AHCNC-CHA for VAN adsorption. FIG. 41 presents the R at 20 mM MA and varying AHCNC-CHA doses. The reduction in R was compensated by increasing the AHCNC-CHA dose to 60 mg mL⁻¹, yielding R~95%. When the MA concentration was above 30 mM, MA carboxylate concentration was higher than the AHCNC-CHA carboxylate content; however, the R still remained at ~60% at 100 mM MA, which may be associated with attractive van der Waals interactions between anionic AHCNC-CHA and nearly neutral MA-adsorbed VAN (ζ-potential=−8±3 mV). This indicates that AHCNC-CHA could efficiently adsorb and remove VAN by inducing phase separation even when carboxylate-bearing competitors are in excess stoichiometric ratios.

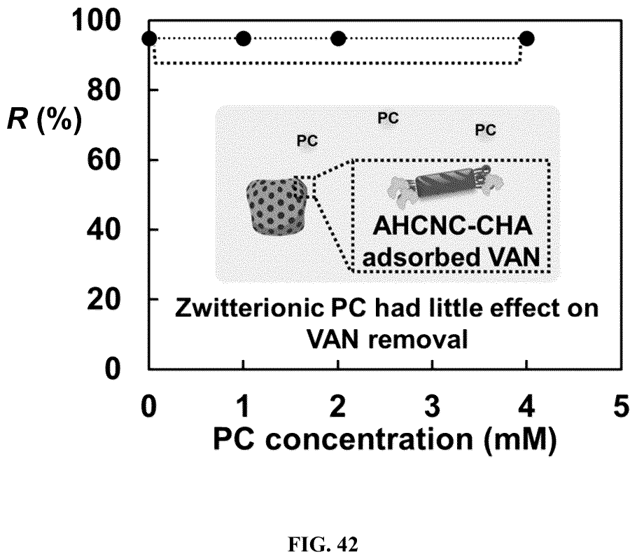

FIG. 42 shows AHCNC-CHA-mediated VAN R at varying PC concentrations, ranging from 1 mM to 4 mM, i.e., the physiological concentration range from FaSSIF to FeSSIF. The R remained at ~95% for all PC concentrations. PC is zwitterionic, which is unable to effectively compete with AHCNC-CHA in VAN binding. These findings confirmed that VAN adsorption to AHCNC-CHA is feasible in the intestines.

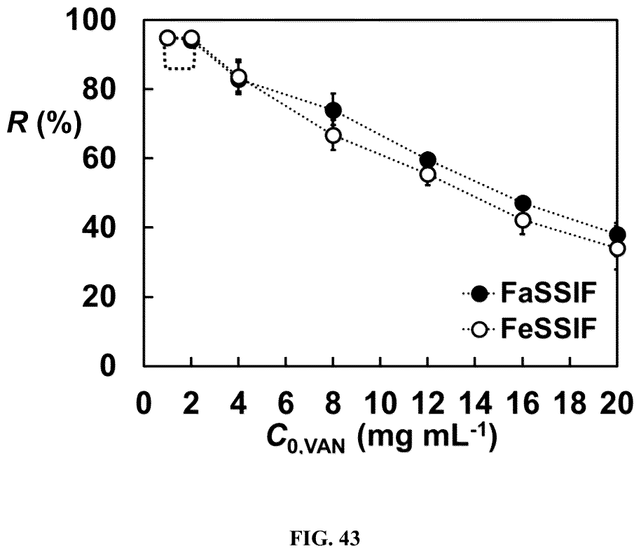

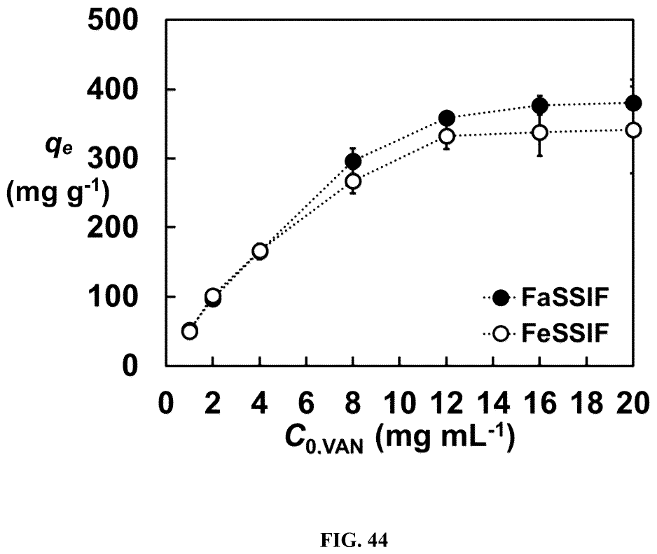

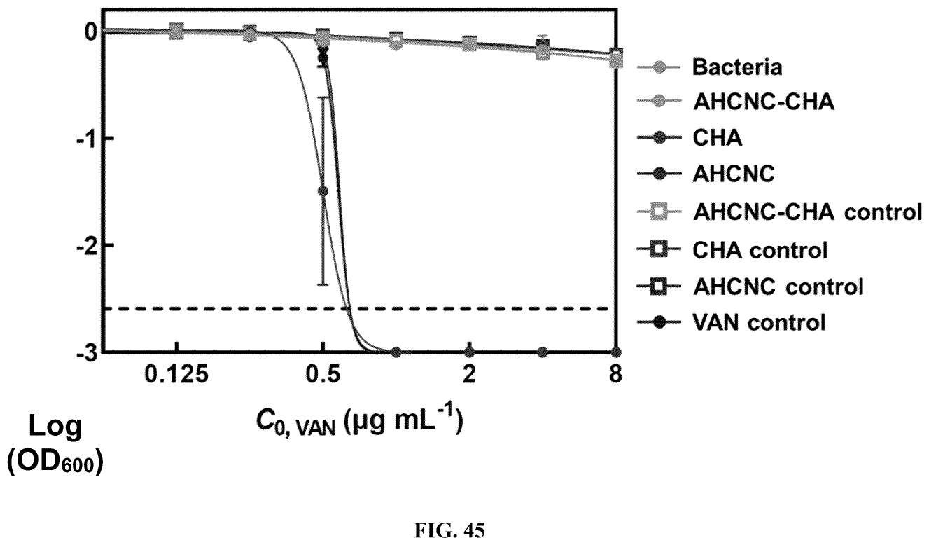

FIG. 43 presents R at varying initial VAN concentrations in the FaSSIF and FeSSIF. R~95% when the initial VAN concentration was <2 mg mL⁻¹ in both conditions; however, R in the FeSSIF was always slightly lower than that in the FaSSIF at initial VAN concentrations of >2 mg mL⁻¹, which was likely a result of the higher BA and MA concentrations in the FeSSIF. FIG. 44 shows qₑ at varying initial VAN concentrations in the FaSSIF and FeSSIF. The qₑ in FaSSIF (~380 mg g⁻¹) and in FeSSIF (~340 mg g⁻¹) were ~50% lower than that in Milli-Q water (~860 mg g⁻¹). The lower qₑ in FaSSIF and FeSSIF was possibly caused by competitive adsorption induced by BA and MA, particularly MA, decreasing R up to ~23% at 19 mM MA (FaSSIF) and ~35% in 55 mM MA (FeSSIF) (FIG. 40).

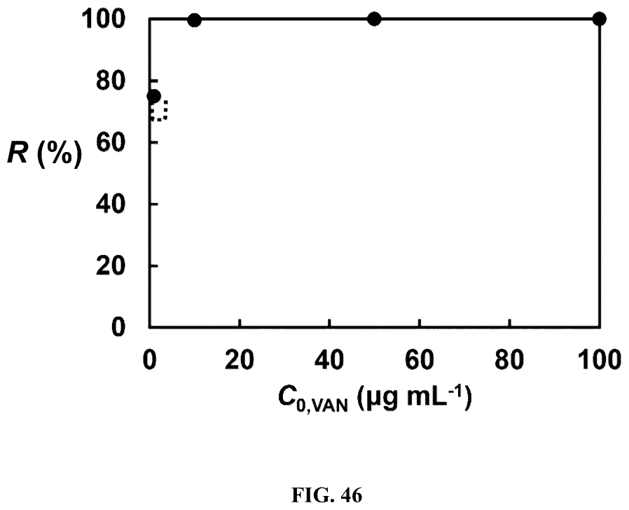

In vitro assessment of VAN activity after AHCNC-CHA-mediated VAN removal: To evaluate the bioactivity of VAN after incubation with AHCNC-CHA, its antibacterial efficacy was tested against VSEfm in the broth microdilution assay at initial VAN concentrations ranging from 0.125 to 8 μg mL⁻¹. FIG. 45 presents the OD₆₀₀ of bacteria following growth in supernatants collected from a VAN solution treated with AHCNC, CHA, or AHCNC-CHA anti-antibiotic independently. The OD₆₀₀ decreased up to 3 orders of magnitude by increasing the initial VAN concentration from 0.5 to 8 μg mL⁻¹ when AHCNC or CHA was used, similar to untreated VAN solution, as these compounds were not able to remove VAN from the supernatants; however, the incubation of AHCNC-CHA anti-antibiotic with VAN resulted in no antibiotic activity against VSEfm. It was observed that AHCNC alone did not induce sufficient colloidal aggregation and phase separation, leaving a large fraction of VAN in the supernatant by which the bacteria were killed. AHCNC-CHA completely captured VAN via phase separation; thus, no antibiotic activity was observed. The AHCNC-CHA removal efficacy at physiologically relevant low VAN concentrations was proved using HPLC analysis. FIG. 46 shows R at initial VAN concentrations below 100 μg mL⁻¹. VAN was almost completely removed at the low antibiotic concentrations.

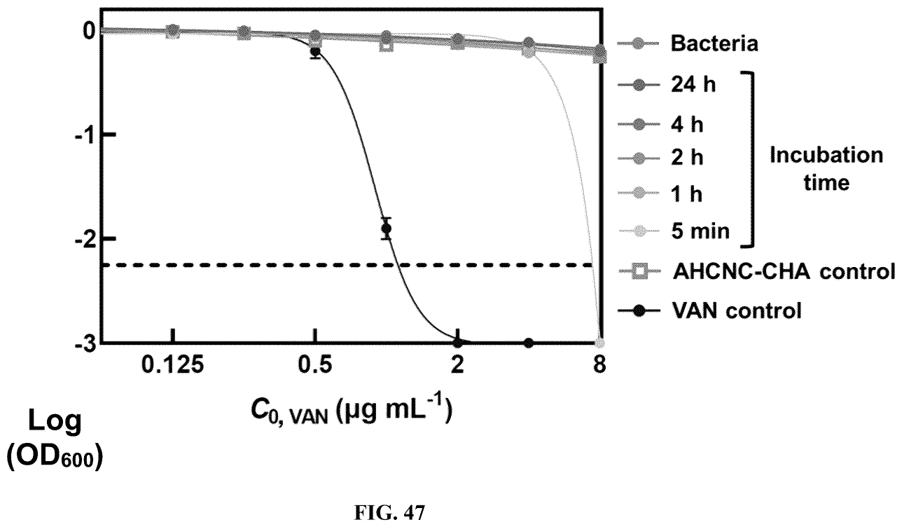

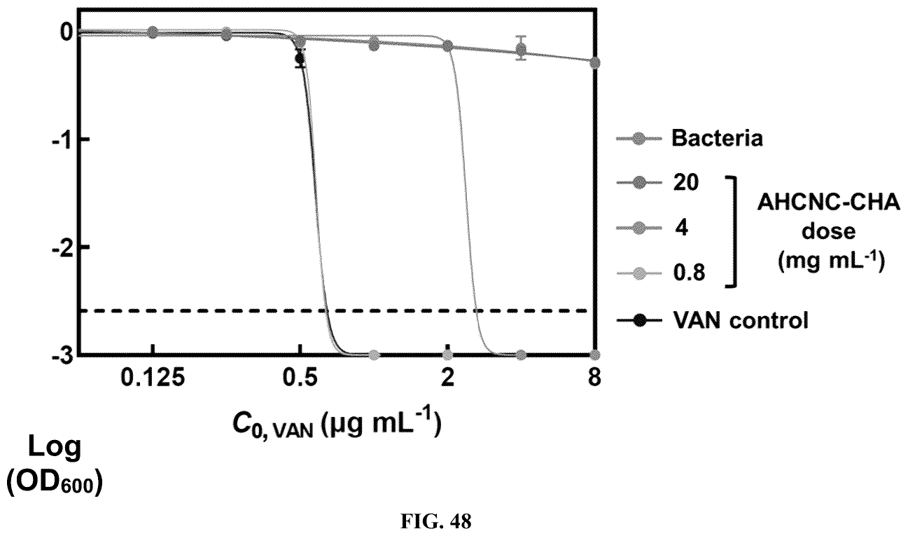

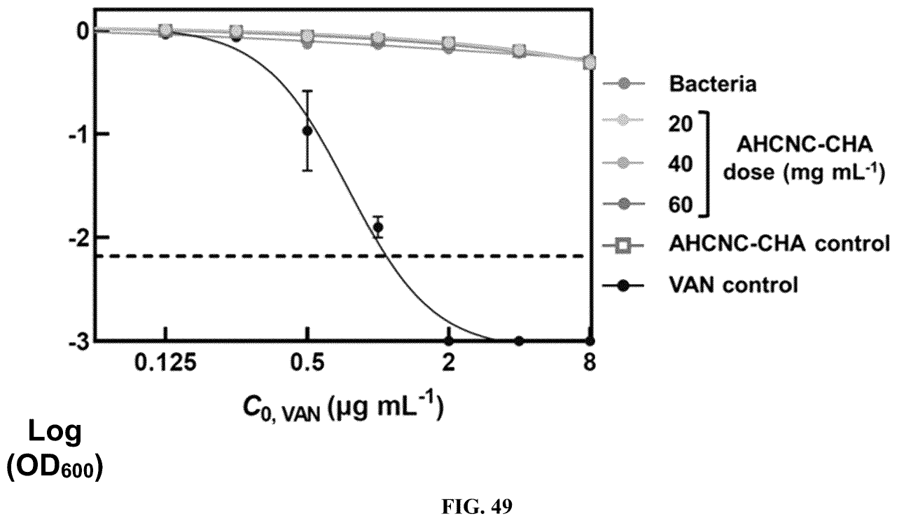

FIG. 47 presents the OD₆₀₀ of bacteria following growth in the supernatants obtained from VAN solutions incubated with AHCNC-CHA anti-antibiotic at varying periods. After 1 h of VAN contact with AHCNC-CHA, no antibiotic activity is detected against VSEfm, implying that the available AHCNC on the surface of CHA swiftly remove VAN. FIG. 48 shows the OD₆₀₀ of bacteria following growth in the supernatants treated with varying AHCNC-CHA doses for 4 h. The available antibiotic in the supernatant decreases by increasing the AHCNC-CHA dose, resulting in less antibiotic activity against VSEfm and an increase in the bacterial density. To evaluate the VAN removal efficacy of AHCNC-CHA exposed to strong acid, mimicking the gastric pH, at physiological VAN concentrations, FIG. 49 presents the OD₆₀₀ of bacteria following growth in supernatants collected from a VAN solution incubated with acid (5 M HCl)-treated AHCNC-CHA for 4 h, implying no antibiotic activity against VSEfm. The results suggest that although the acid-incubated AHCNC-CHA anti-antibiotic undergoes a reduction in VAN (1 mg mL⁻¹) R (shown in FIG. 17), it is able to completely capture VAN in the physiological VAN concentrations.

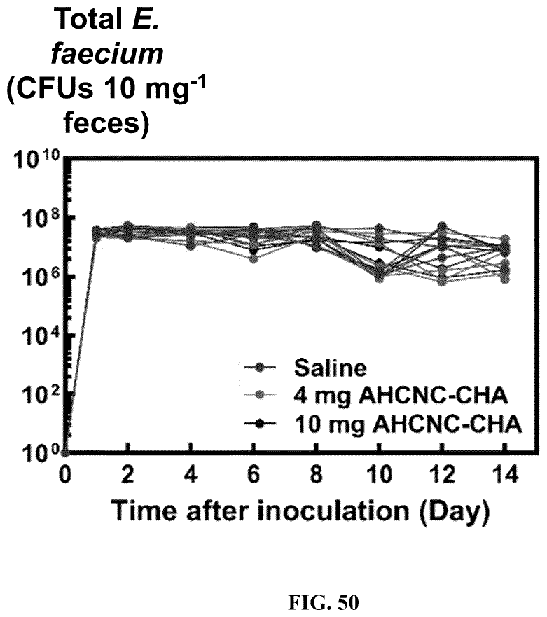

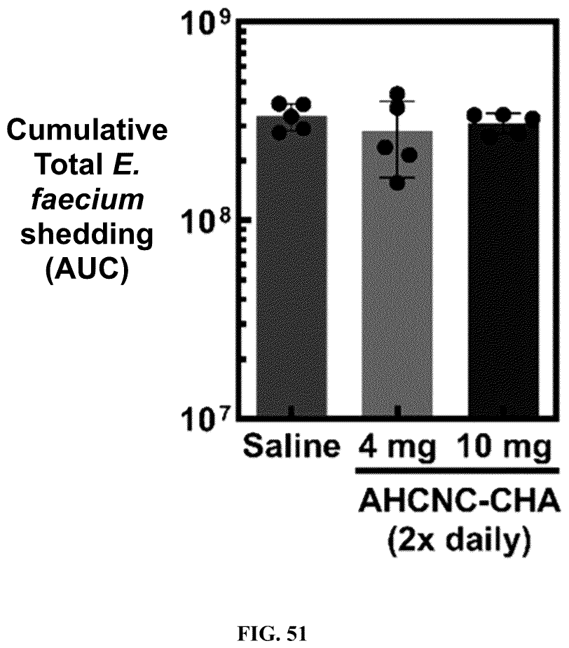

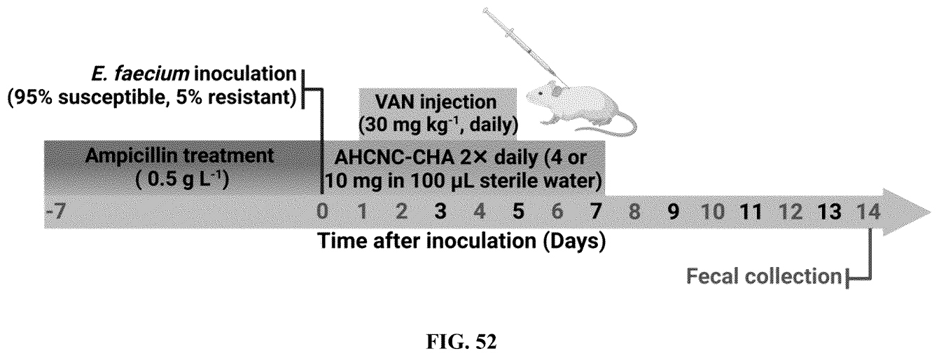

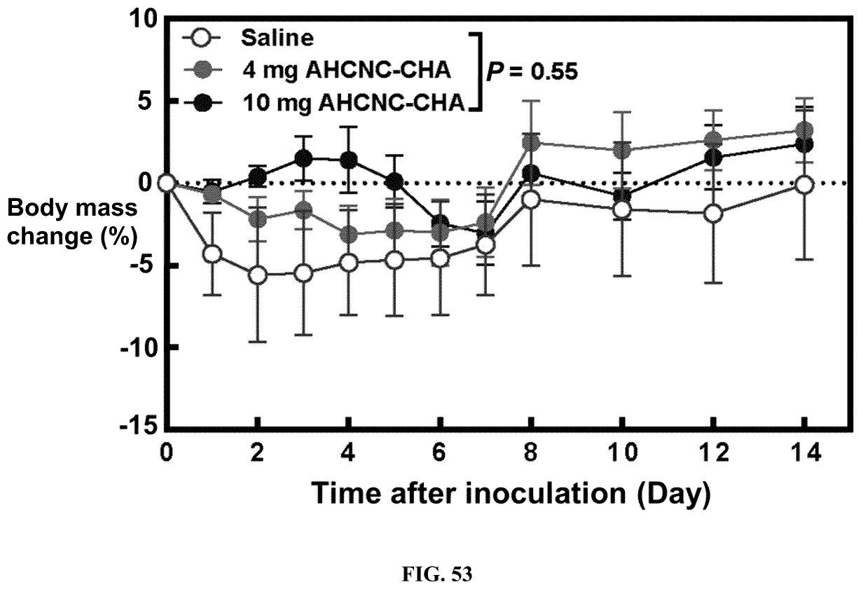

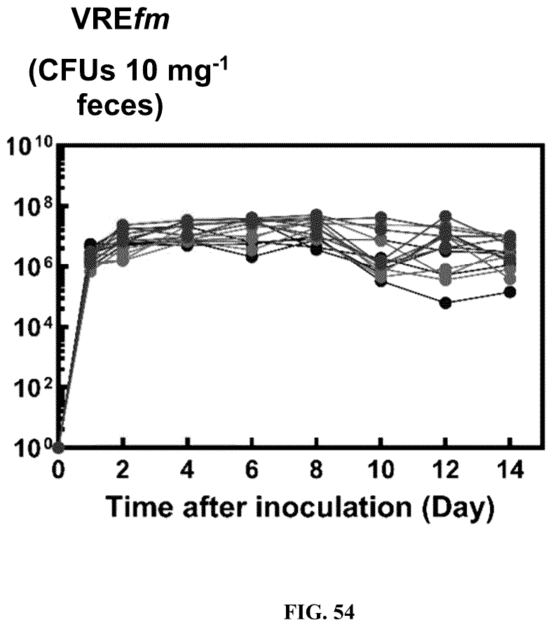

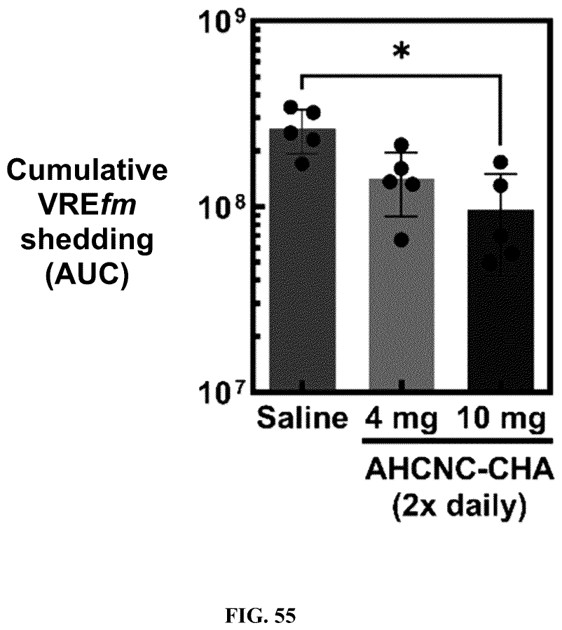

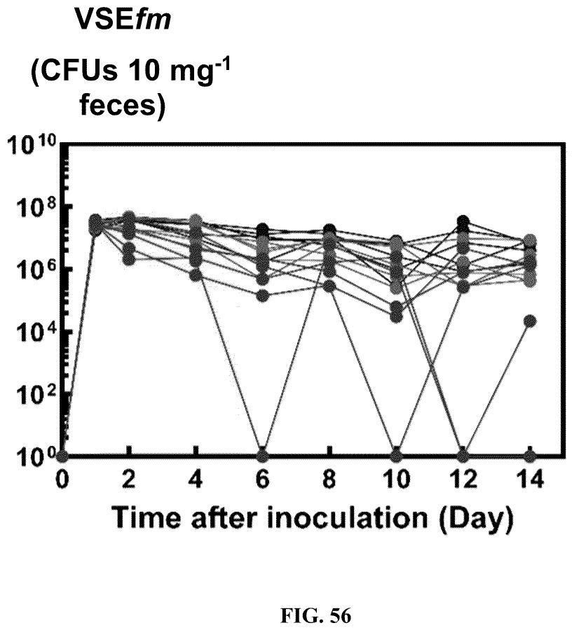

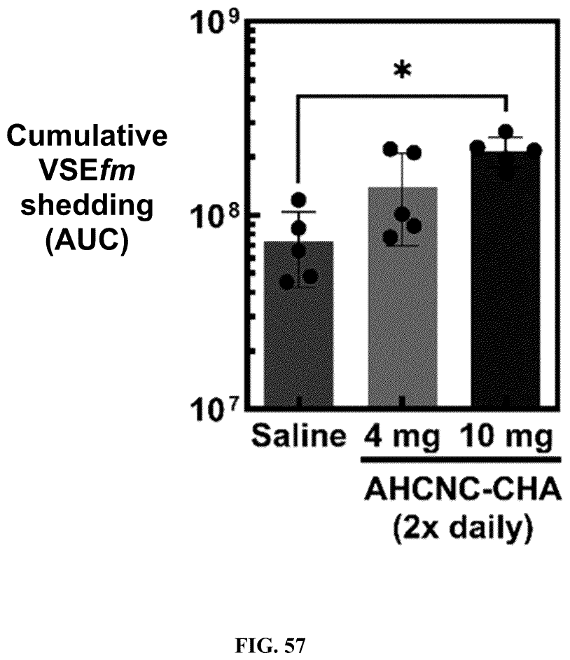

In vivo AHCNC-CHA anti-antibiotic efficacy assessment: To assess the in vivo efficacy of AHCNC-CHA anti-antibiotic in reducing the pressure on resistance selection, a murine model of intestinal E. faecium colonization was used. FIGS. 50-51 show the fecal densities of total E. faecium in mice, treated with saline or either 4 or 10 mg of AHCNC-CHA anti-antibiotic. Prior to bacterial inoculation, no mice had detectable E. faecium in their feces. Following inoculation with a 1:20 ratio of VREfm:VSEfm, treatment of mice with either 4 or 10 mg of AHCNC-CHA anti-antibiotic did not significantly change the total E. faecium in the feces or the body mass of the mice (FIGS. 52-53) relative to the saline-treated controls. FIGS. 54-55 present the fecal densities of VREfm in mice treated with saline or either 4 or 10 mg of anti-antibiotic. In contrast, following subcutaneous VAN injection, mean fecal VREfm density decreased by ~46% and ~63% for the mice treated with 4 mg or 10 mg AHCNC-CHA anti-antibiotic, respectively, relative to the saline-treated controls. These differences were statistically significant in mice treated with 10 mg of AHCNC-CHA. FIGS. 56-57 show the fecal densities of VSEfm in mice treated with saline or either 4 or 10 mg of anti-antibiotic. The decrease in VREfm corresponded with an anti-antibiotic dose-dependent increase in mean VSEfm fecal shedding in mice treated with either 4 mg (by ~90%) or 10 mg (by ~194%) of AHCNC-CHA anti-antibiotic. This again was most pronounced in mice treated with 10 mg of AHCNC-CHA.

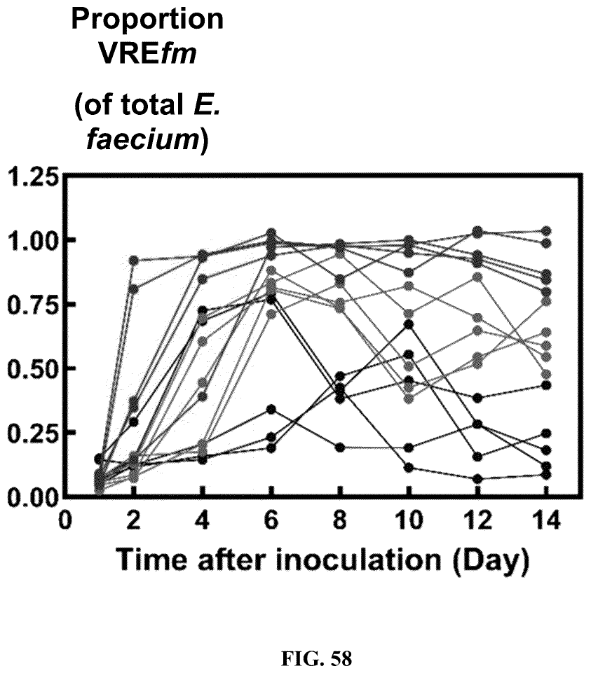

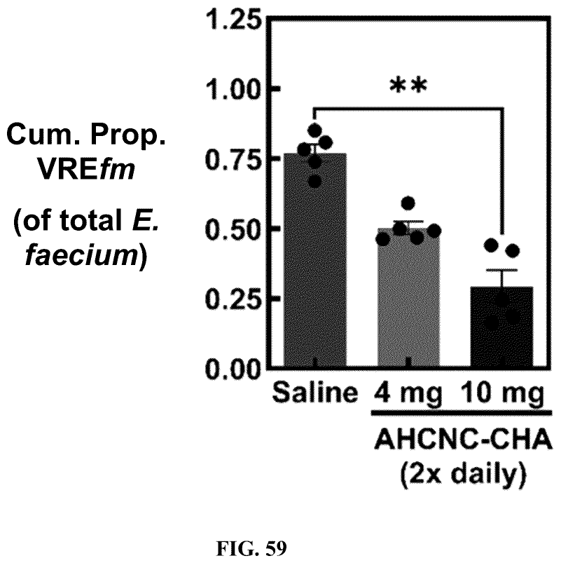

To further explore these relative changes, the proportion of VREfm was calculated for each fecal sample. FIGS. 58-59 present the proportion of VREfm as a function of post-inoculation time and the cumulative portion of VREfm in mice treated with saline or either 4 or 10 mg of AHCNC-CHA anti-antibiotic, respectively. Compared with the saline-treated controls, the mice treated with 10 mg of AHCNC-CHA anti-antibiotic twice daily underwent a significant decrease in the cumulative proportion of fecal VREfm relative to VSEfm (from ~77% for saline treated mice to ~29% for mice treated with 10 mg of AHCNC-CHA anti-antibiotic). Taken together, these data suggested that the AHCNC-CHA effectively sequesters VAN in the murine GI tract, reducing the pressure on resistance selection.

Based on our conducted evaluation work discussed herein, it has been shown that embodiments of our process can remove off-target VAN from the GI tract after intravenous VAN administration, and can effectively prevent the evolution of transmissible VAN-resistant pathogens.

### Example 2

### Materials

The raw material source for HCNC synthesis was sheets of delignified northern bleached softwood kraft (NBSK), received from Domtar Inc., Canada. Sodium chloride (NaCl, >99.5%), sodium (meta) periodate (NaIO₄, >99.0%), calcium chloride dihydrate (CaCl₂·2H₂O, for molecular biology, ≥99.0%), hydrochloric acid (HCl, ACS reagent 37%), sodium hydroxide (NaOH, ACS Reagent, >97%), sodium chlorite (NaClO₂, 80%), hydrogen peroxide (H₂O₂, 30 wt %), ethylene glycol (ReagentPlus®, >99%), hydroxylamine hydrochloride (NH₂OH·HCl, ReagentPlus®, 99%), L-α-phosphatidylcholine (type XVI-E, ≥99.0%), pyrogallol (PG, C₆H₃ (OH)₃, ACS Reagent, >99%), sulfobetaine methacrylate (SBMA, C₁₁H₂₁NO₅S, >95%), 2-aminoethyl methacrylate hydrochloride (AEMA, C₆H₁₁NO₂·HCl, >90%), azobisisobutyronitrile (AIBN, C₈H₁₂N₄, >98%), dimethyl sulfoxide (DMSO, C₂H₆OS, >99.9%), Dulbecco's phosphate buffered saline (DPBS, pH 7.4), sodium dodecyl sulfate (SDS, C₁₂H₂₅NaO₄S, ACS Reagent, >99%), acetic acid (C₂H₄O₂, >50%), bovine serum albumin (BSA, >96%), hen egg white lysozyme (Lyz, >95%), L-lysine monohydrochloride (Lys, >98%), and poly-L-lysin (PLL, 0.1 w/v % in water) were purchased from MilliporeSigma, USA. Vancomycin hydrochloride (USP grade, >95%) was supplied by VWR International, USA. Sodium taurocholate (C₂₆H₄₄NNaO₇S, >95%) was procured from Spectrum Chemical, USA. Maleic acid (C₄H₄O₄, >98%) was purchased from Beantown Chemical Corporation, USA. Fasted-state simulated intestinal fluid (FaSSIF-V2) powder was provided by Biorelevant, UK. Mica sheets (V1 grade) and Krazy glue were supplied by Ted Pella Inc. (USA) and Agar Scientific (UK), respectively. Conductive double-sided adhesive carbon tapes and pin stubs were purchased from Rave Scientific (USA) and Oxford instruments (UK), respectively. Anhydrous ethanol (200 proof) was purchased from KOPTEC, USA. Dialysis tubes (molecular weight cutoff=12-14 kDa) were supplied by Spectrum® Laboratories, USA. Nitrogen (N₂) was provided by Lindle, USA. Ultrapure (Milli-Q) water (resistivity~18.2 MΩ cm at 25° C.) was used in all experiments, unless otherwise stated.

Dulbecco's modified eagle medium (DMEM) and trypsin-ethylenediaminetetraacetic acid (EDTA) solution (0.25%) were purchased from Gibco, USA. Fetal bovine serum (FBS) and antibiotic/antimycotic solution (10,000 U mL⁻¹ penicillin G, 10,000 μg mL⁻¹ streptomycin, 25 μg mL⁻¹ amphotericin B) were supplied by Cytiva, USA. Cell culture flasks and 24 well tissue culture plates were provided by CELLTREAT Scientific Products, USA. The 96 well microplates were purchased from Greiner Bio-One, Austria. PrestoBlue™ Cell Viability Reagent and LIVE/DEAD™ Cell Imaging Kit, containing calcein acetoxymethyl (calcein AM) and BOBO-3 iodide, were supplied by ThermoFisher Scientific, USA.

### Methods

Poly(sulfobetaine methacrylate-co-2-aminoethyl methacrylate) (pSBAE) synthesis: The pSBAE was synthesized according to the literature. Briefly, SBMA (850 mg) and AEMA (49 mg) were dissolved in ultrapure water (50 mL), followed by mixing with a solution of AIBN (15 mg) in DMSO (6 mL). The mixture was then purged with N₂ for 30 min, followed by increasing the mixture temperature to 70° C. in an oil bath to initiate the polymerization. After 20 h of reaction, the mixture was dialyzed (dialysis bag molecular weight cutoff=12-14 kDa) against deionized (DI) water for 3 days to remove unreacted monomers or oligomers. The purified products were frozen at −80° C., followed by sublimating the ice at 0.01 mbar for 48 h using a freeze dryer (FreeZone benchtop, Labconco, USA) and storing at room temperature until further use.

Synthesis of carboxylate group-bearing HCNC:HCNC were synthesized according to an established protocol. The NBSK pulp sheets (1 g) were torn into small pieces and immersed in DI water (100 mL). After one day of soaking, the wet pulp underwent mechanical disintegration using a fruit blender (Cleanblend 3HP, USA) and vacuum-filtered to remove excess water. Subsequently, NaIO₄ (1.32 g) and NaCl (3.8 g) were added to the wet pulp suspension in DI water (total volume=65 mL, including the remaining water in the pulp). To prevent light-induced NaIO₄ deactivation, the reaction beaker was entirely wrapped with aluminum foil, and the mixture was stirred at room temperature for 42 h. To neutralize the unreacted NaIO₄ and terminate the oxidation reaction, ethylene glycol (1 mL) was added to the mixture, and stirring was continued for 15 min. The product of this reaction was dialdehyde-modified cellulose (DAMC) fibers, which were rinsed with DI water using vacuum filtration at least five times.

The never-dried DAMC fibers were further oxidized via a reaction with NaClO₂ (0.48 g) and H₂O₂ (0.48 mL) in DI water (50 mL, including the remaining water in the pulp). The mixture was stirred at room temperature for 12 h while the pH was maintained at ~5.0±0.2 by NaOH solution (0.5 M) addition in the first 5-6 h of reaction. The resulting suspension was then centrifuged at 27000×g for 15 min to precipitate fibers, followed by adding it to 100 mL of DI water and heating at 80° C. for 2 h to break the fibers into HCNC. Afterward, the suspension was centrifuged at 27000×g for 15 min to eliminate non-fibrillated fibers. HCNC were isolated from the supernatant via precipitation using ethanol (0.16 g per 1 g of suspension), followed by centrifugation at 3000×g for 15 min and resuspension in DI water (50 mL). Finally, the HCNC dispersion was purified via dialysis (dialysis bag molecular weight cutoff=12-14 kDa) against DI water for 1 day.

Aldehyde group content measurement: An oxime titration method was conducted to quantify the aldehyde group content of DAMC (the intermediate oxidized fibrils). Never-dried DAMC fibrils (20 mg) were suspended in ultrapure water (50 mL), and the pH was decreased to 3.5 using HCl (0.1 M). Then, 10 mL of a 5 wt. % NH₂OH·HCl solution (pH adjusted to 3.5 using a 0.1 M NaOH solution) was added. When the aldehyde groups reacted with NH₂OH·HCl during the oxime reaction, HCl was released. The decrease in pH was compensated via adding a 10 mM NaOH solution using an automatic titrator (Metrohm 907 Titrando, USA) at a rate of 0.1 mL min-1. The volume of NaOH consumed to reach the initial pH of 3.5 was then used to calculate the DAMC aldehyde content.

Carboxylate group content measurement: Carboxylate content of HCNC was quantified by conductometric titration. HCNC (20 mg) were dispersed in ultrapure water (140 mL), and 2 mL of a NaCl solution (20 mM) was added to the dispersion. Then, pH was adjusted to 3 via adding a HCl solution (0.1 M). Using the automatic titrator, the dispersion was titrated with a NaOH solution (10 mM) at a rate of 0.1 mL min-1 until the pH reached ~11. The carboxylate content was calculated from the middle part of titration curves, corresponding to the weak acid.

Hydro Hydrodynamic size and ζ-potential measurements: To measure the HCNC hydrodynamic size, dynamic light scattering (DLS) spectroscopy was carried out using a Zetasizer Nano series instrument (Malvern Inc., UK) at a scattering angle of 90° and temperature~25° C. A 0.1% w/v HCNC dispersion was prepared using ultrapure water, and 70 μL of it was transferred into a low-volume quartz cuvette (ZEN2112, Malvern, UK). The Z-average value, i.e., cumulants mean, was reported as the hydrodynamic equivalent size.

To measure ζ-potential, the HCNC electrophoretic mobility was determined using the Zetasizer Nano series instrument at room temperature and pH=5.7. About 1 mL of a 0.1% w/v HCNC dispersion was pipetted into disposable folded capillary cells (Malvern, UK) for measurement. Since the HCNC have a rod-like shape, Oshima's mobility expression was used to calculate the ζ-potential value. As κa<1 (K is the Debye-Hückel parameter, and a is the HCNC radius~2.5 nm, obtained from the AFM image analysis), the Henry's function ƒ(κa)→0.5, and Equation (5) yields the ζ-potential using the electrophoretic mobility:

$${μ}_{av} = \frac{{μ}_{‖} + 2 {μ}_{⊥}}{3} = \frac{{ε}_{r} {ε}_{0}}{3 η} ζ [ 1 + 2 f ( κ a ) ] \qquad ( 5 )$$

where μ_{∥} and μ_{⊥} are the electrophoretic mobilities of rod-like particles in the parallel and perpendicular direction to the electric field, respectively, Er and co are the relative dielectric permittivity of the solvent and the vacuum permittivity, respectively, and η is the viscosity of electrolyte solution. Note that the ionic strength was calculated via considering the sodium counter ions of HCNC carboxylate groups.

Atomic force microscopy: To investigate HCNC morphology, a MultiMode® atomic force microscope (Bruker Dimension Icon I, USA) was used. To prepare the HCNC sample for imaging, freshly cleaved mica was first adhered to a stainless-steel disc using the Krazy glue and pre-coated with a PLL solution (10 μL, 0.1% w/v). After 10 min, the excess PLL was rinsed with ultrapure water. Then, 10 μL of a HCNC dispersion (0.1% w/v) was pipetted on the mica sheet, air-dried overnight, rinsed gently with 100 μL of ultrapure water 5 times, and again air-dried overnight. The imaging was conducted at the PeakForce tapping mode using a silicon nitride probe (Bruker ScanAsyst-Air, USA) with a cantilever that had the following characteristics: thickness=650 nm, length=115 μm, width=25 μm, resonance frequency=70 kHz, and spring constant=0.4 N m⁻¹. The scan size and rate were 2 μm×2 μm and 0.5 Hz, respectively. Length and width (height) of ~50 particles in the AFM image (n=1) were measured by the Gwyddion software (Version 2.49, accessed via Penn State Materials Characterization Laboratory, MCL).

Formulation of ViPZ-HCNC composition: To synthesize ViPZ-HCNC and maximize the VAN removal percentage and selectivity, varying ViPZ-HCNC formulations were developed via an optimized PG-assisted pSBAE deposition procedure, as presented in Table 1. Briefly, VAN (2.2 or 3.3 mg) and DPBS (10% v/v) were added to a HCNC dispersion (final concentration=2 mg mL⁻¹), followed by adding PG (final concentration=8 mg mL⁻¹) and pSBAE (final concentration=16 or 40 mg mL-1). The reaction proceeded at 45° C. via continuous stirring. After 12 h, the ViPZ-HCNC were separated via centrifugation at 3000×g for 15 min and washed with a mixture of 2% v/v acetic acid and 2% w/v SDS (1:1 volume ratio) 4-5 times until no absorbance peak was detected for VAN at a maximum peak of wavelength (λₘₐₓ)~280 nm in the supernatants using an ultraviolet-visible (UV-vis) spectrophotometer (Tecan Infinite M Plex, Switzerland). The imprinted ViPZ-HCNC were rinsed with 70% v/v ethanol at least 5 times and with ultrapure water at least 10 times to remove residual SDS and acetic acid. The purified materials were frozen at −80° C., followed by sublimating the ice at 0.01 mbar for 48 h using the freeze dryer and storing at room temperature for further experiments. For comparison, two controls were considered: (i) a non-imprinted polymerized zwitterionic HCNC (NI-PZ-HCNC) was prepared using the same protocol but without VAN immobilization and (ii) a VAN imprinted HCNC with 1.5:1 molar ratio of NH₃⁺:COO⁻ (Vi-HCNC-1.5-0) was prepared without pSBAE polymerization. To calculate VAN concentration in the solutions, VAN calibration lines were obtained via UV-vis spectrophotometry.

TABLE 1
Varying ViPZ-HCNC formulations, used to maximize VAN
(1 mg mL⁻¹) removal percentage and selectivity.

| | | | | | NH₃⁺:COO⁻ | |
|---|---|---|---|---|---|---|
| | | HCNC | PG | VAN | molar | pSBAE |
| Compound | Sorbents† | (mg mL⁻¹) | (mg mL⁻¹) | (mg mL⁻¹) | ratio‡ | (mg mL⁻¹) |
| A | ViPZ-HCNC1-16 | 1 | 8 | 2.2 | 1:1 | 16 |
| B | ViPZ-HCNC1-40 | 1 | 8 | 2.2 | 1:1 | 40 |
| C | ViPZ-HCNC1.5-16 | 1 | 8 | 3.3 | 1.5:1 | 16 |
| D | ViPZ-HCNC1.5-40 | 1 | 8 | 3.3 | 1.5:1 | 40 |
| E | NI-PZ-HCNC | 1 | 8 | 0 | 0 | 16 |
| F | Vi-HCNC-1.5-0 | 1 | 8 | 3.3 | 1.5:1 | 0 |

†The values of x and y in the ViPZ-HCNCx-y sorbent names refer to NH₃⁺:COO⁻ molar ratio and pSBAE concentration used during the synthesis process, respectively. Note that NH₃⁺:COO⁻ molar ratio and pSBAE concentration for compound E are 0 and 16 mg mL⁻¹, respectively, which are not shown for simplicity.
‡NH₃⁺ and COO⁻ groups are related to VAN and HCNC, respectively.

Field emission scanning electron microscopy (FE-SEM) imaging: Freeze-dried HCNC, pSBAE, or ViPZ-HCNC were mounted on pin stubs using double-sided adhesive carbon tape and coated with a 3 nm-thick indium layer using a vacuum sputtering instrument (Leica sputter coating EM ACE 600, USA). The samples were then imaged using FE-SEM (ThermoFisher Scientific Apreo S, USA) with a T2 upper in-lens detector at an accelerating voltage of 5.0 kV and a beam current of 13 pA (for HCNC) or 6.3 pA (for pSBAE and ViPZ-HCNC). The magnification for HCNC was 100,000×, while for pSBAE and ViPZ-HCNC, it was 250,000×.

ViPZ-HCNC-mediated biomolecule (VAN, BSA, Lyz, or Lys) removal: All the removal experiments were conducted in batch processes. Biomolecule (VAN, BSA, Lyz, or Lys) stock solutions (2 mg mL⁻¹) were independently prepared by dissolving each biomolecule (20 mg) in ultrapure water (10 mL). The stock solutions were then separately diluted using ultrapure water to a final concentration of 1 mg mL⁻¹ and a final volume of 1 mL, including the volume of NaOH (0.5 M) to adjust the pH=6.5. Varying sorbents (5 mg) were added to the VAN solutions (1 mL), vortexed for 5 min, and placed on a nutating mixer (Fisherbrand, USA) to agitate at 60 rpm for 2 h. The samples were then centrifuged at 5000×g for 5 min, and the supernatants were collected and analyzed via UV-vis spectrophotometry to quantify the concentration of unadsorbed VAN at λₘₐₓ=280, Lyz at λₘₐₓ=280 nm, BSA at λₘₐₓ=278 nm, or Lys at λₘₐₓ=479 nm. Equation (7) and Equation (8) were used to calculate the removal percentage (R, %) and the equilibrium removal capacity (qₑ, mg mL⁻¹) of VAN, Lyz, BSA, or Lys,

$$R \% = \frac{( {c}_{0} - {c}_{e} )}{{c}_{0}} \times 100 \% \qquad ( 7 )$$

$${q}_{e} = \frac{( {c}_{0} - {c}_{e} )}{m} \times V \qquad ( 8 )$$

where C₀ (mg mL⁻¹) is the initial concentration of each biomolecule, Cₑ (mg mL⁻¹) is the equilibrium concentration of each biomolecule after removal, m (g) is the mass of sorbent, and V (mL) is the total solution volume. Note that the lower detection limit for VAN concentration using UV-vis spectrophotometry was ~0.05 mg mL⁻¹. When VAN Cₑ was below the detection limit, it was considered to be 0.05 mg mL⁻¹. Therefore, any R value~95% calculated using this consideration is likely underestimated.

Imprinting factor (IF) calculation: To evaluate the VAN recognition ability of ViPZ-HCNC, IF was calculated based on Equation (9)

$$IF = \frac{{q}_{e , ViPZ - HCNC}}{{q}_{e , NI - PZ - HCNC}} \qquad ( 9 )$$

where q_{e, ViPZ-HCNC} is the equilibrium VAN removal capacity of ViPZ-HCNC, and qₑ,NI-PZ-HCNC is the equilibrium VAN removal capacity of NI-PZ-HCNC.

Effect of calcium ions (Ca²⁺) on ViPZ-HCNC-mediated VAN removal: To investigate the effect of Ca²⁺ concentrations on the VAN-selectivity of ViPZ-HCNC, VAN removal experiments were conducted at 0, 10, or 100 mM of Ca²⁺. VAN solutions with a final concentration of 1 mg mL⁻¹ and a final volume of 1 mL were prepared via diluting a VAN stock solution with ultrapure water, including the volume of NaOH (0.5 M), used to adjust the pH to 6.5. Varying sorbents including ViPZ-HCNC or pSBAE (5 mg) were independently added to the VAN solutions (1 mL) containing varying Ca²⁺ concentrations, vortexed for 1 min, and placed on a nutating mixer, operated at 60 rpm. After 2 h of incubation, the samples were centrifuged at 5000×g for 5 min, and the supernatants were subsequently analyzed via UV-vis spectrophotometry at λₘₐₓ=280 nm to determine the free VAN concentration. Equation (7) was used to calculate VAN removal percentage (R_{VAN}) at varying Ca²⁺ concentrations.

Effect of ViPZ-HCNC dose on VAN removal: To study the effect of ViPZ-HCNC dose on VAN removal, VAN solutions with a final concentration of 1 mg mL⁻¹ and a final volume of 1 mL were prepared by diluting a VAN stock solution, including the volume of NaOH (0.5 M) used to adjust the pH to 6.5. Varying doses of ViPZ-HCNC (5, 10, 15, or 20 mg) were added to the VAN solutions, vortexed for 1 min, and placed on the nutating mixer to agitate at 60 rpm. Following a 2 h incubation period, the samples were centrifuged at 5000×g for 5 min, and the supernatants were analyzed using the UV-vis spectrophotometer at λₘₐₓ=280 nm to obtain the free VAN concentration. Equation (7) was used to calculate R_{VAN} at varying ViPZ-HCNC doses.

Effect of incubation time on ViPZ-HCNC-mediated VAN removal: To investigate the effect of incubation time on VAN removal, ViPZ-HCNC (20 mg) was added to the VAN solutions (1 mg mL⁻¹, pH~6.5), vortexed for 1 min, and placed on the nutating mixer to agitate at 60 rpm. At varying incubation times (10 min, 30 min, 1 h, 2 h, 3 h, 4 h, 5 h, or 6 h), the samples were centrifuged at 5000×g for 5 min, and the supernatants were analyzed using the UV-vis spectrophotometer at λₘₐₓ=280 nm to obtain the free VAN concentration. Equation (7) was used to calculate R_{VAN} at varying incubation times.

Effect of initial VAN concentration on ViPZ-HCNC-mediated VAN removal: To examine the effect of initial VAN concentration on VAN removal, ViPZ-HCNC (20 mg) was added to the VAN solutions (1 mg mL⁻¹, pH~6.5), vortexed for 1 min, and placed on a nutating mixer at 60 rpm. After 2 h of incubation, the samples were centrifuged at 5000×g for 5 min, and the supernatants were analyzed using the UV-vis spectrophotometer at λₘₐₓ=280 nm to obtain the free VAN concentration. Equation (7) and Equation (8) were used to measure R_{VAN} and qₑ, respectively.

ViPZ-HCNC-mediated VAN removal in the SIF: To evaluate the ViPZ-HCNC-mediated VAN removal in media mimicking the intestinal fluids, we followed standard procedures to generate two SIF types: fasted state SIF (FaSSIF) and fed state SIF (FeSSIF). For the FaSSIF solution, NaOH pellets (1.392 g), maleic acid (2.22 g), and NaCl (4.01 g) were dissolved in ultrapure water (0.99 L). The pH was adjusted to 6.5 using varying concentrations of NaOH solutions (0.1 M, 0.5 M, and 1.0 M), and the total solution volume was increased to 1 L using ultrapure water. Then, the FaSSIF-V2 powder (1.79 g) was added to the solution and stirred at room temperature for 1 h, yielding the FaSSIF. To make the FeSSIF, sodium taurocholate (8.25 g) was added to the FaSSIF (250 mL) and stirred at room temperature until it was entirely dissolved. Afterward, L-α-phosphatidylcholine (2.95 g) was added, stirred for 4 h until a clear solution was obtained, and the final volume was adjusted to 1 L using the FaSSIF solution.

To conduct VAN removal experiments in the FaSSIF or FeSSIF, VAN solutions with varying concentrations (1-8 mg mL⁻¹) were prepared by diluting a VAN stock solution (20 mg g⁻¹ VAN in ultrapure water) with FaSSIF or FeSSIF. The final pH was adjusted to 6.5 using a NaOH solution (0.5 M), and the total solution volume for each removal experiment was 1 mL. ViPZ-HCNC (20 mg) were added to the VAN solutions, vortexed for 1 min, and placed on a nutating mixer at 60 rpm. Following a 2 h incubation, the samples were centrifuged at 5000×g for 5 min, and the supernatants were analyzed using the UV-vis spectrophotometer at λₘₐₓ=280 nm to assess the free VAN concentrations. Equation (7) and Equation (8) were used to obtain R_{VAN} and qₑ, respectively.

Cell viability and metabolic activity assessments: NIH/3T3 murine fibroblast cells (ATCC, USA) were cultured in complete culture media containing DMEM, supplemented with 10% v/v FBS and 1% antibiotic/antimycotic solutions. The cell culture was conducted using T75 flasks, containing 10⁴ cells per cm², at 37° C. and 5% v/v CO₂ in a cell culture incubator (Eppendorf C170i, Germany), and the media were refreshed every other day. Cells were passaged when they reached ~80% confluency.

Before conducting cell viability and metabolic activity assays, a trypsin-EDTA (0.25%) solution was used to detach cells from the cell culture flask. Then, 10⁴ cells were seeded in each well of 24 well tissue culture plates. After one day of culture in supplemented DMEM, the media was fully replaced with fresh, complete culture media, containing varying concentrations of ViPZ-HCNC (0, 0.001, 0.01, 0.1, or 1 mg mL⁻¹) in each well. Every other day, the culture media was fully removed and replaced with ViPZ-HCNC-supplemented complete culture media. The metabolic activity of cells treated with varying concentrations of ViPZ-HCNC was measured using a PrestoBlue™ Cell Viability Reagent. The metabolic activity was measured on days 1, 4, and 7 after introducing ViPZ-HCNC-supplemented culture media to the cells. Briefly, PrestoBlue solution was mixed with FBS-free DMEM at a 1:10 volume ratio to prepare the PrestoBlue working solution. The culture media in each well was then replaced with the PrestoBlue working solution (1 mL), and cells were incubated under 5% v/v carbon dioxide (CO₂) at 37° C. in the cell culture incubator. After 3 h, 100 μL of media was pipetted into each well of 96 well microplates, and the fluorescence intensity was measured using a microplate reader (Tecan Infinite M Plex, Switzerland) at an excitation wavelength of 530 nm and an emission wavelength of 590 nm. The fluorescence intensity was corrected with respect to the background signal, measured from the PrestoBlue working solution incubated in a cell-free well of a 24 well tissue culture plate under the same condition.

Cell viability was measured using the LIVE/DEAD™ Cell Imaging Kit. On day 7 after incubating cells in the media containing ViPZ-HCNC, 1 mL of calcein AM (1 μM) was added to 1 μL of BOBO-3 iodide and mixed to prepare a stock solution. The working solution was then prepared by adding 1 mL of DPBS to the stock solution. Subsequently, 200 μL of the working solution was added to each well of a 24 well tissue culture plate, containing the cells treated with varying ViPZ-HCNC concentrations (0, 0.001, 0.01, 0.1, or 1 mg mL⁻¹) in the culture media. After 30 min, samples were imaged using a Leica microscope (DMi8 THUNDER Imager 3D Cell Culture, Leica Microsystems, Germany) at the excitation/emission wavelengths of 470/510 nm for live cells and 550/610 nm for dead cells. Images were imported to Fiji ImageJ software (version 1.54f, NIH, USA) and converted to 8-bit images, followed by thresholding and calculating the area of live or dead cells. Cell viability was reported as the ratio of the area occupied by live cells to the total area occupied by both live and dead cells.

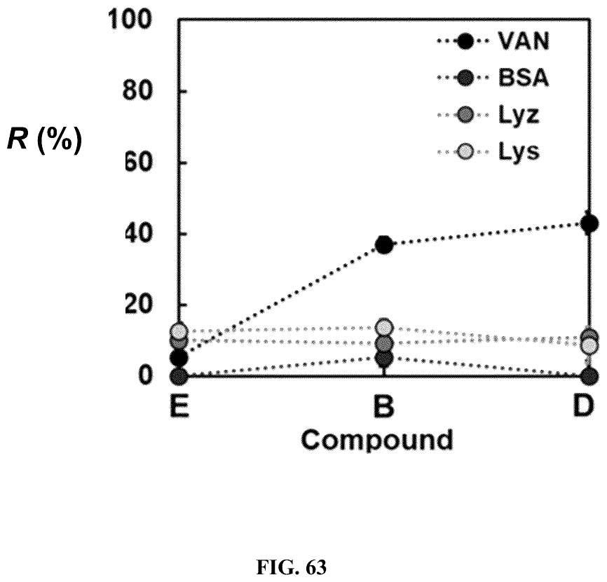

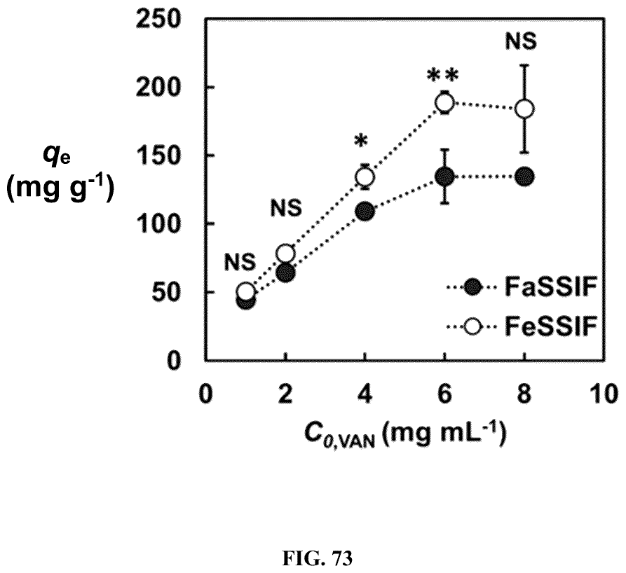

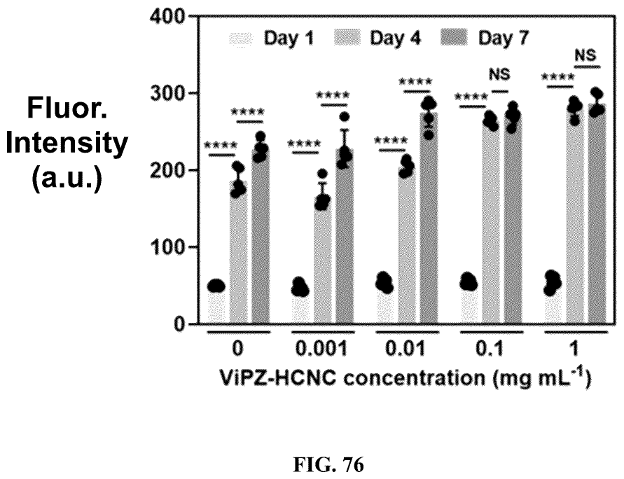

Statistical Analyses: Data were collected with at least three independent repeats and reported as mean±standard deviation (SD) unless otherwise stated. Statistical analyses were performed using GraphPad Prism software (version 10.0.3). One-way (FIGS. 63 and 76) analysis of variance (ANOVA), followed by the Tukey post-hoc test, was used for three distinct groups, and Student's t-test (FIG. 73) was conducted for two distinct groups. The symbols *, **, ***, and **** represent p-values of <0.05, <0.01, <0.001, and <0.0001, respectively. The p-values≥0.05 were considered non-significant (NS).

### Results and Discussion

ViPZ-HCNC development: Antifouling ViPZ-HCNC with specific binding cavities for VAN removal is developed via integrating a zwitterionic polymer (pSBAE) as an anti-nonspecific protein adsorption component, HCNC as a template, and PG as a crosslinker. FIG. 60 presents the ViPZ-HCNC development by pSBAE attachment to VAN-decorated HCNC via PG-mediated polymerization. In the first step, VAN is adsorbed to the HCNC template through the electrostatic interaction between cationic VAN molecules and anionic HCNC. The pSBAE is then reacted with purpurogallin, generated from PG (8 mg mL⁻¹), on the VAN-bearing HCNC.

The purpurogallin has a carbonyl group that can react with compounds bearing amine groups. To enable one-step pSBAE and PG polymerizations, pSBAE are synthesized via copolymerization of SBMA and AEMA, to provide amino groups for reacting with purpurogallin. Previously, PG self-polymerization at a PG concentration of 8 mg mL⁻¹ has enabled the pSBAE attachment to surfaces. Due to the presence of amine groups on pSBAE and aldehyde groups on HCNC, a Schiff base reaction between pSBAE and HCNC is likely to occur. In addition, we hypothesize that the polymerized pSBAE on HCNC surface may prevent non-specific adsorptions, facilitating selective VAN removal by ViPZ-HCNC. The pSBAE coatings have previously inhibited mammalian cell adhesion and have significantly reduced protein (e.g., fibrinogen) adsorption. After imprinting and polymerization, a surfactant (SDS) in an acetic acid solution is used to remove the adsorbed VAN from the polymerized HCNC. This step is necessary to detach or degrade imprinted molecules, creating specific binding cavities, as previously reported for the removal of lysozyme from zwitterionic polymer chain-assisted lysozyme imprinted core-shell carbon microspheres.

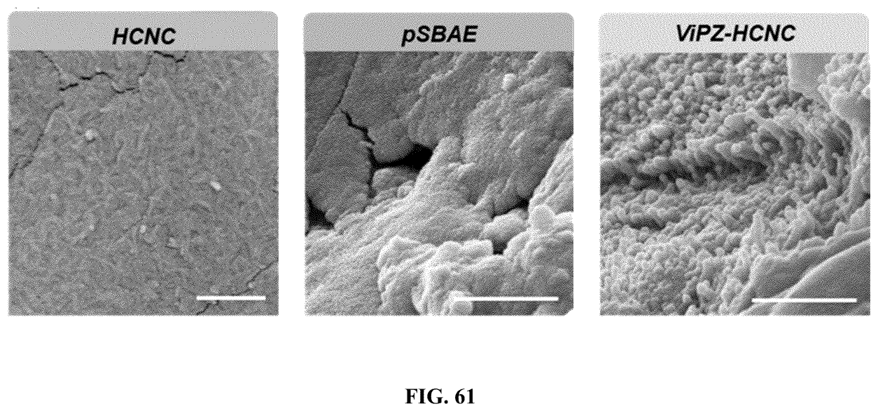

To investigate the surface morphology of HCNC, zwitterionic pSBAE, and ViPZ-HCNC, FE-SEM is conducted, as shown in FIG. 61. The rod-like structures of aggregated HCNC are visible in the left panel. The freeze-dried pSBAE has an almost smooth and dense surface (middle panel). When pSBAE is polymerized with PG on VAN-templated HCNC, brush-like and spherical features are observed (right panel). These polymer coatings may impart antifouling properties to ViPZ-HCNC via introducing hydrophilic functional groups and forming a hydration layer. In another study, the pSBAE/polydopamine coating shows pSBAE assemblies with spherical morphologies with a thickness of 3.8 μm. ViPZ-HCNC-assisted anti-VAN therapy may enable specific VAN binding to prevent the resistance evolution in the gut microbiome. If VAN remains in the intestine, it can drive VAN susceptible bacteria toward the resistant mutants, which may enter the bloodstream and cause serious infections. ViPZ-HCNC can be used to develop sensors capable of detecting VAN among other biomolecules such as enzymes and proteins, as well as electrolytes. Additionally, it may be used as a sorbent for the selective removal of VAN from water and contaminated wastewater. Given the cellulose and PG degradability, the ViPZ-HCNC are likely to degrade in the environment, thereby minimizing their ecological footprint after disposal.

HCNC synthesis and characterization: The HCNC, templates for the ViPZ-HCNC, are synthesized and thoroughly characterized with respect to the carboxylate group content, particle size, ζ-potential, and morphology. FIG. 2 shows a scheme of HCNC synthesis, involving cellulose fibrils, DAMC, and dicarboxylate-modified cellulose fibrils. Cellulose fibrils are partially oxidized using NaIO₄ to produce DAMC as an intermediate material for HCNC synthesis. After conducting a controlled chlorite-mediated oxidation reaction with DAMC fibrils, a fraction of the aldehyde groups is converted to carboxylate groups, yielding dicarboxylate-modified cellulose fibrils, and HCNC are yielded following a heat treatment, ethanol-mediated precipitation, and centrifugation. The evidence of amorphous cellulose chains (hairs), protruding from the crystalline ends has been described in our review papers. The crystalline properties of more oxidized HCNC than ours have been previously investigated using X-ray diffraction (XRD), confirming the characteristic peaks of cellulose Iβ at 2θ=15.3°, 16.5°, and 22.6° in the nanocrystals pattern. FIG. 4 shows a representative pH titration curve for DAMC fibrils. The amount of NaOH used to neutralize the HCl released as a result of NH₂OH. HCl-aldehyde reaction is used to quantify the aldehyde content (6.1±0.3 mmol g⁻¹). FIG. 5 shows a representative conductometric titration curve of HCNC, bearing 3.2±0.3 mmol of carboxylate groups per gram of HCNC. The hydrodynamic size (equivalent diameter) of HCNC is 144±16 nm at pH~5.7 and ionic strength~3.2 mM. The HCNC ζ-potential at the same condition is −30±5 mV, confirming the negatively charged carboxylate groups on the nanoparticles. FIG. 3 shows a representative AFM image of HCNC, confirming that HCNC have a rod-like or whisker-shape morphology (length~112±19 nm and width~5±2 nm, obtained from AFM height measurements).

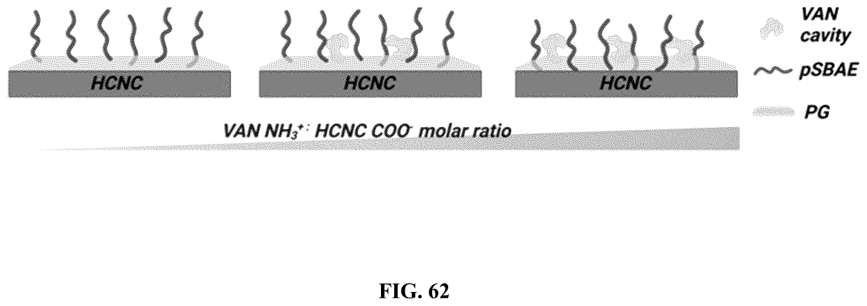

Optimization of ViPZ-HCNC composition: The ViPZ-HCNC composition is optimized to maximize R_{VAN} and VAN selectivity against biologically relevant competitive biomolecules and ions. To this end, varying formulations of ViPZ-HCNC are prepared based on Table 1 to investigate the effects of imprinted VAN (tailored via varying VAN NH₃⁺:HCNC COO⁻ ratios) and pSBAE concentrations on the VAN removal percentage of ViPZ-HCNC in complex media. To validate our hypothesis that imprinting VAN on a template via the MIP strategy enables the selective removal of VAN from mixed ion/biomolecule solutions, we vary the VAN concentration in the formulations. FIG. 62 presents a scheme of ViPZ-HCNC VAN cavities as a function of VAN NH₃⁺: HCNC COO⁻ molar ratio, implying that increasing the imprinted VAN molecules creates additional cavities for specific VAN binding.

FIG. 63 presents the removal of VAN and other biomolecules, R_{VAN}, R_{BSA}, R_{Lyz}, and R_{Lys}, measured individually for compounds E, B, and D that are prepared using varying VAN NH₃⁺:HCNC COO molar ratios (0, 1:1, 1.5:1). Note that this molar ratio is tailored via changing VAN concentration and maintaining HCNC concentration constant. R_{BSA}, R_{Lyz}, and R_{Lys} are all below ~15% for all the compounds; however, R_{VAN} increases from ~5±2% to ~43±3% when VAN NH₃⁺:HCNC COO⁻ molar ratio increases from 0 (compound E) to 1.5 (compound D) as a result of the increased number of VAN specific binding sites on ViPZ-HCNC.

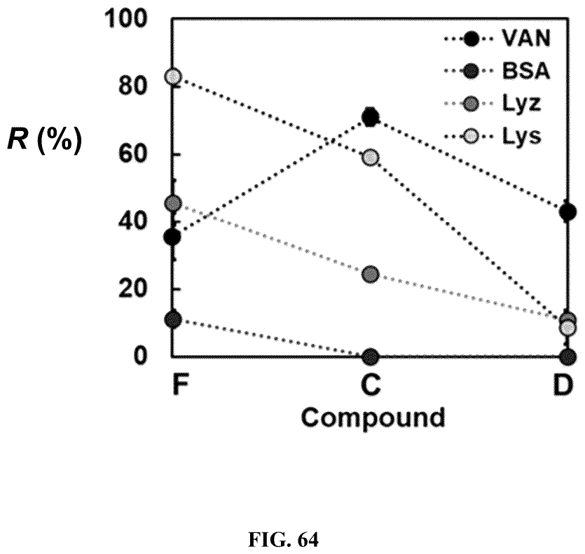

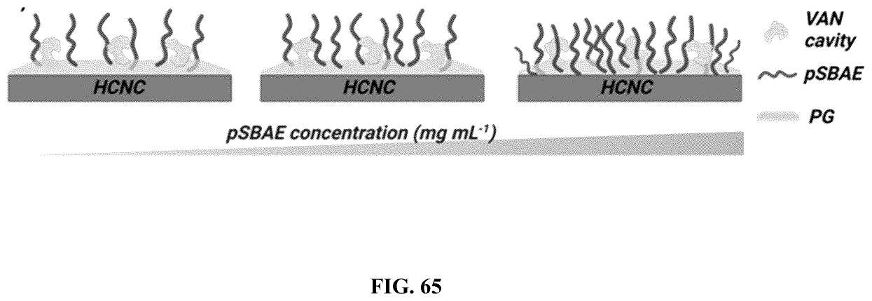

To validate the hypothesis that pSBAE polymerization on HCNC may prevent non-specific adsorption of biomolecules and enable selective VAN removal by ViPZ-HCNC, we vary the pSBAE concentration and determine R. FIG. 64 presents R_{VAN}, R_{BSA}, R_{Lyz}, and R_{Lys}, measured individually for compounds F, C, and D that are prepared by varying pSBAE concentrations at a constant VAN NH₃⁺:HCNC COO⁻ molar ratio (1.5:1). By increasing the pSBAE concentration from 0 (compound F) to 40 mg mL⁻¹ (compound D), R_{BSA}, R_{Lyz}, and R_{Lys} monotonically decrease and reach less than ~9%, implying that ViPZ-HCNC repel the biomolecules as a result of pSBAE coating. On the other hand, R_{VAN} increases from ~36±7% to 71±2% by increasing the pSBAE concentration from 0 (compound F) to 16 mg mL⁻¹ (compound C) and decreases to 43±3% by further increasing the pSBAE concentration to 40 mg mL⁻¹ (compound D). This reduction may be attributed to the excessive zwitterionic functional groups of pSBAE, which may weaken the interactions between ViPZ-HCNC and VAN at pSBAE concentrations>16 mg mL⁻¹. FIG. 65 presents a scheme of ViPZ-HCNC, prepared at varying pSBAE concentrations and a constant VAN NH₃⁺:HCNC COO⁻ molar ratio. By increasing the pSBAE concentration, ViPZ-HCNC is rendered zwitterionic, which repel non-specific ions while binding VAN via the templated VAN cavities.

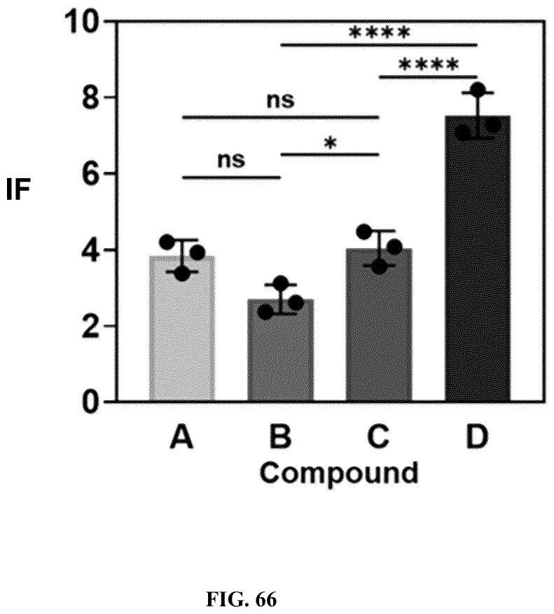

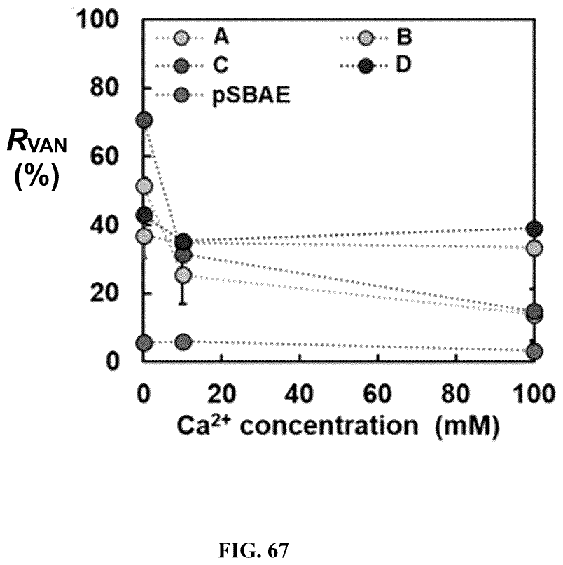

To quantify VAN recognition ability of ViPZ-HCNC sorbents, FIG. 66 presents IF for compounds A, B, C, and D, prepared at varying VAN NH₃⁺:HCNC COO⁻ molar ratios and pSBAE concentrations. At a fixed pSBAE concentration (40 mg mL⁻¹), the IF increases by increasing VAN NH₃⁺:HCNC COO⁻ molar ratio (1:1 for compound B and 1.5:1 for compound D), likely because of forming more VAN-specific cavities. Additionally, at a fixed VAN NH₃⁺:HCNC COO molar ratio (1.5:1), IF increases by increasing the pSBAE concentration from 16 mg mL⁻¹ (compound C) to 40 mg mL⁻¹ (compound D). Among all ViPZ-HCNC sorbents, compound D has the highest IF (7.5±0.5). To examine the selectivity of ViPZ-HCNC sorbents against Ca²⁺, FIG. 67 shows the R_{VAN} of compounds A-D and pSBAE at varying Ca²⁺ concentrations. The R_{VAN} of compounds A and C, both prepared using 16 mg mL⁻¹ of pSBAE, decreases from ~52±2% to 14±7% and 71±1% to 15±1%, respectively, by increasing Ca²⁺ concentrations from 0 to 100 mM. This indicates that Ca²⁺ causes significant competitive adsorption, likely because of the effect of divalent Ca²⁺ on the hydrogen-bonding networks of water molecules. However, the R_{VAN} of compounds B and D, both prepared at 40 mg mL⁻¹ of pSBAE, decreases only ~3% by increasing Ca²⁺ concentrations from 0 to 100 mM. The results show that increasing the polymerized pSBAE on the HCNC surface reduces non-specific Ca²⁺ adsorption, likely as a result of the strong hydration effect of zwitterions. The pSBAE-mediated R_{VAN} is significantly low (~6%) irrespective of Ca²⁺concentration because of the lack of VAN cavities. Together, compound D (ViPZ-HCNC1.5-40), prepared at a VAN NH₃⁺:HCNC COO⁻ molar ratio of 1.5:1 and a pSBAE concentration of 40 mg mL⁻¹ is selected as the optimal compound, yielding the highest R_{VAN} at 100 mM of Ca²⁺ compared with other compounds.

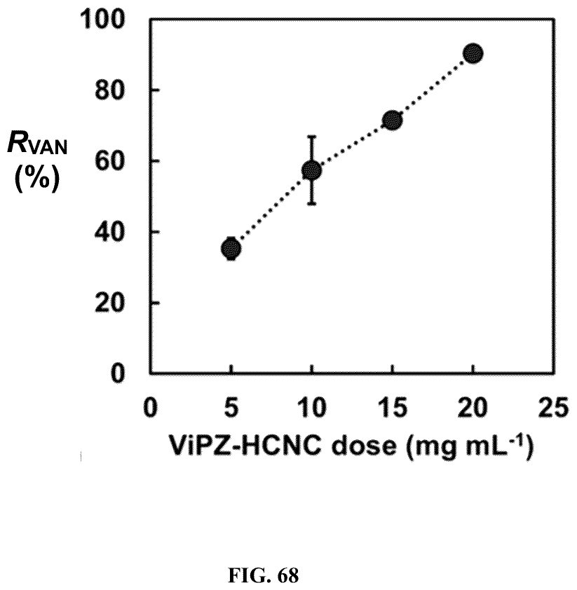

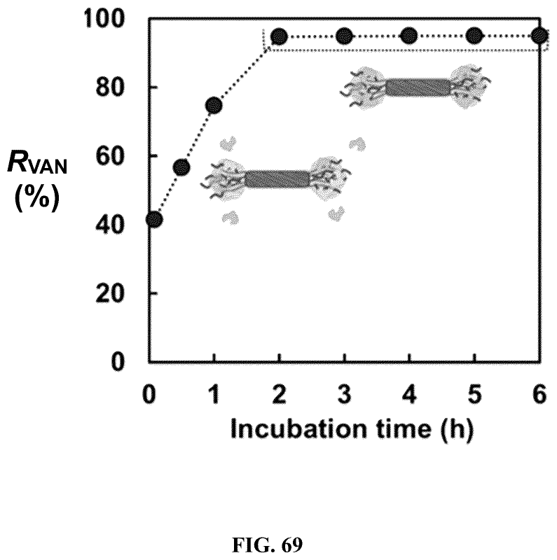

Effect of ViPZ-HCNC dose and incubation time on VAN removal: The optimal ViPZ-HCNC, i.e., ViPZ-HCNC1.5-40, is selected for further investigations, including sorbent dose-dependent VAN removal, VAN removal kinetics, and VAN removal at varying initial VAN concentrations in ultrapure water and SIF. FIG. 68 presents the R_{VAN} of ViPZ-HCNC at varying sorbent doses and a constant VAN initial concentration (C_{0,VAN})=1 mg mL⁻¹. R_{VAN} increases monotonically from ~35±3% to ~90±2% by increasing the ViPZ-HCNC dose from 5 mg mL⁻¹ to 20 mg mL⁻¹, confirming that VAN removal depends on the sorbent dose. FIG. 69 shows the R_{VAN} (C_{0,VAN}=1 mg mL⁻¹) of ViPZ-HCNC (20 mg mL⁻¹) at varying incubation times. R_{VAN} reaches ~41±1% within 5 min and increases to ~95% after ~2 h of incubation. The required incubation time for cavity-mediated VAN removal may be a result of multiple cooperative weak interactions, such as van der Waals, hydrogen bonding, and/or IT-IT interactions. These are the common interactions involved in the adsorption of biomolecules, e.g., BSA, to high-recognition PIMs, such as silicon oxide (SiO₂) @γ-methacryloxypropyltri-methoxysilane (MPS) @MIPs-macromolecularly functional monomer (MFM) and template-imprinted nanostructured surfaces with polysaccharide-enabled cavities, reaching the equilibrium at ~2 h.

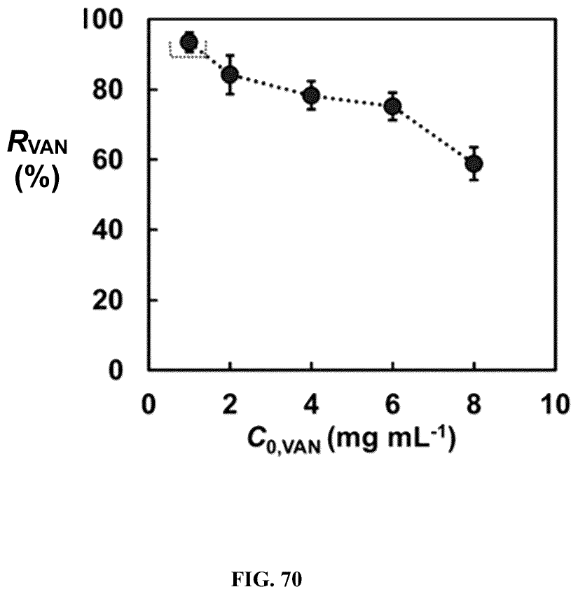

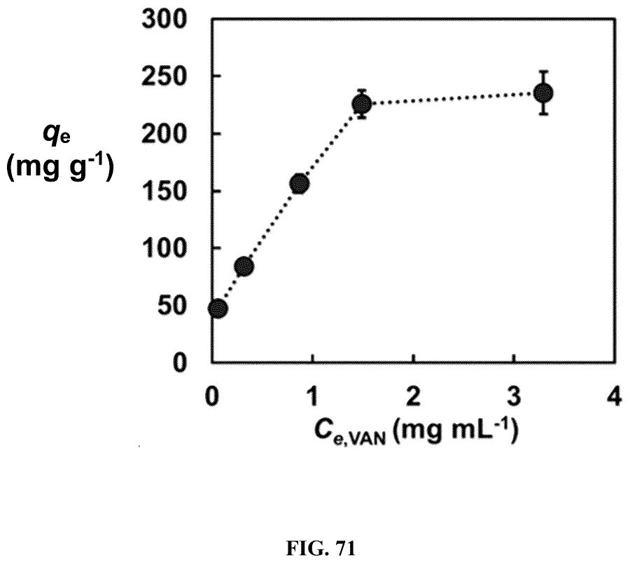

Effect of initial VAN concentration of ViPZ-HCNC-mediated VAN removal: To examine the VAN removal efficacy of ViPZ-HCNC, FIG. 70 presents R_{VAN} of ViPZ-HCNC (20 mg mL⁻¹) at varying C_{0,VAN}. R_{VAN} decreases from ~94±4% to ~59±5% by increasing the C_{0,VAN} from 1 mg mL⁻¹ to 8 mg mL⁻¹. The decrease in R_{VAN} is caused by the saturation of VAN binding cavities on ViPZ-HCNC as VAN concentration increases at a constant sorbent dose. To study the VAN adsorption isotherm, FIG. 71 shows the qₑ of ViPZ-HCNC (20 mg mL⁻¹) at varying equilibrium VAN concentrations (C_{e,VAN}). qₑ increases as C_{e,VAN} increases to 1.5 mg mL⁻¹, reaching qₑ~225±12 mg g⁻¹. Compared with the literature reporting VAN removal using DADA-coated PEG microparticles (qₑ~510 mg g⁻¹ after 24 h incubation), ViPZ-HCNC adsorb VAN significantly faster, but with a lower removal capacity.

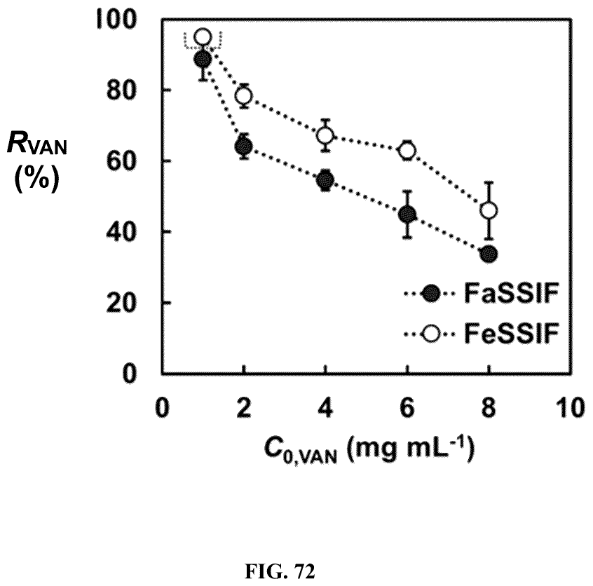

ViPZ-HCNC-mediated VAN removal in SIF: To investigate the effect of intestinal fluids on ViPZ-HCNC-mediated VAN removal, FIGS. 72 and 73 present the R_{VAN} and qₑ of ViPZ-HCNC (20 mg mL⁻¹) at C_{0,VAN} varying from 1 to 8 mg mL⁻¹ in the FaSSIF and FeSSIF, respectively. R_{VAN}~89±6% in the FaSSIF and ~95% in the FeSSIF at C_{0,VAN}~1 mg mL⁻¹, implying that the competitive biomolecules (e.g., bile acid and maleic acid) in the intestinal fluids do not significantly affect ViPZ-HCNC at physiologically relevant low VAN concentrations. By increasing C_{0,VAN} from 1 to 8 mg mL⁻¹, R_{VAN} decreases to ~37±1% in the FaSSIF and ~46±8% in the FeSSIF as a result of sorbent saturation. The qₑ of ViPZ-HCNC is ~135 mg g⁻¹ in the FaSSIF and ~188 mg g⁻¹ in the FeSSIF, which are ~42% and ~20% less than that in ultrapure water (~235±19 mg g⁻¹, FIG. 71), respectively.

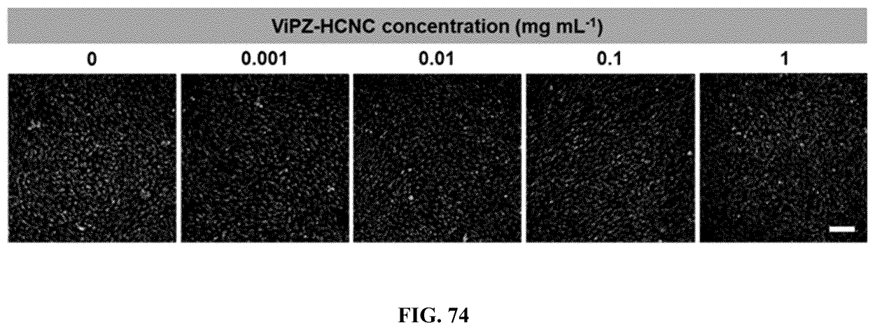

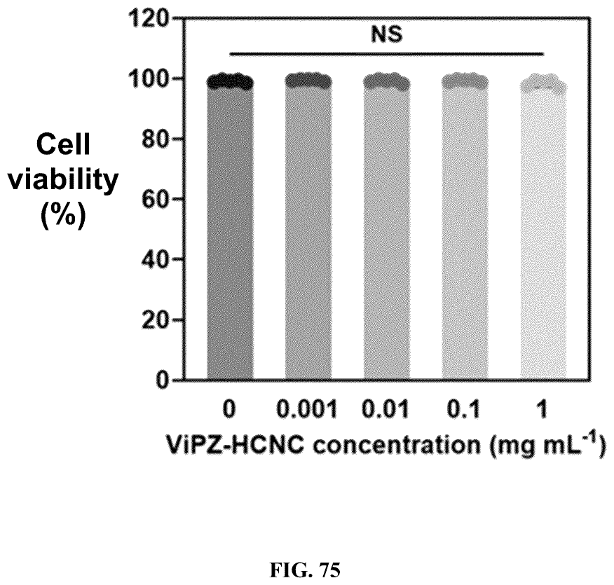

Cell viability and metabolic activity assessments: The toxicity of ViPZ-HCNC (compound D) at varying concentrations is assessed in vitro via incubating varying doses of sorbent with cultured NIH/3T3 murine fibroblast cells. FIGS. 74 and 75 show fluorescence microscopy images following Live/Dead cell staining on day 7 and the corresponding cell viability, respectively, after one week of culturing cells in the media, containing varying ViPZ-HCNC concentrations (0-1 mg mL⁻¹). The cell viability is ~100% at all ViPZ-HCNC concentrations, and no significant difference is observed between the control and varying ViPZ-HCNC concentrations. FIG. 76 shows the metabolic activity of cells over 7 days of culture in the media, containing varying ViPZ-HCNC concentrations. An increasing trend in metabolic activity of cells is observed for ViPZ-HCNC concentrations, ranging from 0 to 0.01 mg mL⁻¹ over 7 days of culture. At ViPZ-HCNC concentrations of 0.1 and 1 mg mL⁻¹, although the metabolic activity significantly increases from day 1 to day 4, no significant increase is observed from day 4 to day 7. Additionally, the metabolic activity of cells treated with varying concentrations of ViPZ-HCNC remains similar to or higher than those cultured in the ViPZ-HCNC-free media, indicating that the sorbent does not have toxicity against the cells within the concentration range investigated here.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the apparatus and methods of using and making the same disclosed herein have been discussed and illustrated, it is to be distinctly understood that the invention is not limited thereto but may be otherwise variously embodied and practiced within the scope of the following claims.

What is claimed is:

1. A method of forming an antibiotic removal agent, the method comprising:
providing hairy cellulose nanocrystals;
adsorbing a target antibiotic compound onto the hairy cellulose nanocrystals via electrostatic interactions;
polymerizing a zwitterionic polymer around the antibiotic-adsorbed hairy cellulose nanocrystals; and
removing the target antibiotic compound via a surfactant or solvent to form the antibiotic removal agent.

2. The method of claim 1, wherein the hairy cellulose nanocrystals comprise cellulose bodies bearing cellulose chains protruding therefrom, wherein the cellulose chains are functionalized with anionic groups.

3. The method of claim 2, wherein the anionic groups comprise carboxylate groups.

4. The method of claim 1, wherein the hairy cellulose nanocrystals comprise cellulose bodies bearing cellulose chains protruding therefrom, wherein the cellulose chains are functionalized with cationic groups.

5. The method of claim 1, wherein the hairy cellulose nanocrystals comprise cellulose bodies bearing cellulose chains protruding therefrom, wherein the cellulose chains are functionalized with amphiphilic groups.

6. The method of claim 1, wherein the hairy cellulose nanocrystals comprise cellulose bodies bearing cellulose chains protruding therefrom, wherein the cellulose chains are functionalized with zwitterionic groups.

7. The method of claim 1, wherein the agent is configured to be administered to a gastrointestinal (GI) tract of a patient, such that when the GI tract includes the target antibiotic compound, the agent is configured to effectively remove the one or more antibiotic compounds.

8. The method of claim 1, wherein the target antibiotic compound is selected from the group consisting of include B-lactams, aminoglycosides, chloramphenicol, glycopeptides, quinolones, oxazolidinones, sulfonamides, tetracyclines, macrolides, ansamycins, streptogramins, lipopeptides, and combinations thereof.

9. The method of claim 8, wherein the target antibiotic compound is Vancomycin (VAN).

10. The method of claim 1, wherein the zwitterionic polymer is poly(sulfobetaine) (pSBAE).

11. An antibiotic removal agent formed from the method of claim 1.

12. A method of forming an antibiotic removal agent, the method comprising:
providing a functionalized biopolymer, wherein the functionalized biopolymer is selected from the group consisting of polysaccharide, protein, polynucleotides, polypeptides, and mixtures thereof;
adsorbing a target antibiotic compound onto the functionalized biopolymer via electrostatic interactions;
polymerizing a zwitterionic polymer around the antibiotic-adsorbed biopolymer; and
removing the target antibiotic compound via a surfactant or solvent to form the antibiotic removal agent.

13. The method of claim 12, wherein the biopolymer is functionalized with anionic groups.

14. The method of claim 13, wherein the anionic groups comprise carboxylate groups.

15. The method of claim 12, wherein the agent is configured to be administered to a gastrointestinal (GI) tract of a patient, such that when the GI tract includes the target antibiotic compound, the agent is configured to effectively remove the one or more antibiotic compounds.

16. The method of claim 12, wherein the target antibiotic compound is selected from the group consisting of include B-lactams, aminoglycosides, chloramphenicol, glycopeptides, quinolones, oxazolidinones, sulfonamides, tetracyclines, macrolides, ansamycins, streptogramins, lipopeptides, and combinations thereof.

17. The method of claim 16, wherein the target antibiotic compound is Vancomycin (VAN).

18. The method of claim 12, wherein the zwitterionic polymer is poly(sulfobetaine) (pSBAE).

19. An antibiotic removal agent formed from the method of claim 12.
